SEQUENCE LISTING (102 sequences)

SEQ ID NO: 1 - protein, 463 aa, synthetic construct
MALPVTALLLPLALLLHAARPDIVLTQSPASLAVSPGQRATITCRASESVTILGSHLIHWYQQKPGQPPKLLINLASNVNTGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTDYSINWVRQAPGKGLEWVGWINTETREPAYAYDFRGRFTFSADTSKSMAYLQINSLRAEDTAVYYCALDYTYGMDYWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 2 - protein, 463 aa, synthetic construct
MALPVTALLLPLALLLHAARPDIVLTQSPASLAVSPGQRATITCRASESVTILGSHLIHWYQQKPGQPPKLLINLASNVNTGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTDYSINWVRQAPGKGLEWVGWINTETREPAYAYDFRGRFTFSADTSKSMAYLQINSLRAEDTAVYYCALDYTYGMDYWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRVRFSRSADAPAYQQGQNQLYNELNLGRREEYDVLDRRRGRDPEMGGRPQRRRNPQEGLYNELQRDRMAEAYSEIGMRGERRRGRGHDGLYQGLSTATRDTYDALHMQALPPR

SEQ ID NO: 3 - protein, 463 aa, synthetic construct
MALPVTALLLPLALLLHAARPDIVLTQSPASLAVSPGQRATITCRASESVTILGSHLIHWYQQKPGQPPKLLINLASNVNTGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTDYSINWVRQAPGKGLEWVGWINTETREPAYAYDFRGRFTFSADTSKSMAYLQINSLRAEDTAVYYCALDYTYGMDYWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRVAFSRSADAPAYQQGQNQLYNELNLGRREEYDVLDARRGRDPEMGGAPQRRANPQEGLYNELQADAMAEAYSEIGMAGERRRGAGHDGLYQGLSTATADTYDALHMQALPPR

SEQ ID NO: 4 - protein, 463 aa, synthetic construct
MALPVTALLLPLALLLHAARPDIVLTQSPASLAVSPGQRATITCRASESVTILGSHLIHWYQQKPGQPPKLLINLASNVNTGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTDYSINWVRQAPGKGLEWVGWINTETREPAYAYDFRGRFTFSADTSKSMAYLQINSLRAEDTAVYYCALDYTYGMDYWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRVDFSRSADAPAYQQGQNQLYNELNLGRREEYDVLDDRRGRDPEMGGDPQRRDNPQEGLYNELQDDDMAEAYSEIGMDGERRRGDGHDGLYQGLSTATDDTYDALHMQALPPR

SEQ ID NO: 5 - protein, 463 aa, synthetic construct
MALPVTALLLPLALLLHAARPDIVLTQSPASLAVSPGQRATITCRASESVTILGSHLIHWYQQKPGQPPKLLINLASNVNTGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTDYSINWVRQAPGKGLEWVGWINTETREPAYAYDFRGRFTFSADTSKSMAYLQINSLRAEDTAVYYCALDYTYGMDYWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRVEFSRSADAPAYQQGQNQLYNELNLGRREEYDVLDERRGRDPEMGGEPQRRENPQEGLYNELQEDEMAEAYSEIGMEGERRRGEGHDGLYQGLSTATEDTYDALHMQALPPR

SEQ ID NO: 6 - DNA, 1392 bp, synthetic construct
  source 1..1392: mol_type = other DNA
atggcactgccagtcacagcactgctgctgccactggcactgctgctccacgccgccagacccgacatcgtgctgacacagagcccagcaagcctggccgtgagcccaggccagagagccacaatcacatgcagagccagcgagtccgtgacaatcctgggctcccacctgatccactggtaccagcagaagcccggacagccacccaaactgctgatcaacctggccagcaacgtgaacacaggcgtgccagccagattcagcggcagcggcagcggaaccgacttcaccctgacaatcagctcagtggaaccagaagacaccgccaactactactgcctgcagagcagaacactgcccagaaccttcggccaaggcaccaaggtcgaaatcaagggctccacaagcggcagcggaaagcccggaagcggcgagggaagcaccaagggccagatccagctggtgcagagcggccccgaactgaagaaacccggcggaagcgtgaaaatctcctgcaaggccagcggctacaccttcaccgactacagcatcaactgggtgcgccaggccccaggcaagggcctggaatgggtcggctggatcaacaccgagacaagagagcccgcctacgcctacgacttcagaggcagattcaccttcagcgcagacacaagcaagagcatggcctacctgcagatcaactccctgagagcagaggacaccgccgtctactactgcgccctggactacacctacggcatggactactggggccagggcaccctcgtgaccgtgtccagcttcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacagagtgaagttcagcagatccgccgacgcaccagcctaccagcagggacagaaccagctgtacaacgagctgaacctggggagaagagaagagtacgacgtgctggataagcggagaggcagagaccctgagatgggcggcaagccccaaagacggaagaacccacaagagggcctgtacaacgagctgcagaaagacaagatggccgaggcctacagcgagatcggaatgaagggcgagcgcagaagaggcaagggacacgacggactgtaccagggcctgagcacagccaccaaggatacctacgatgccctgcacatgcaggccctgccaccaagatga

SEQ ID NO: 7 - DNA, 1392 bp, synthetic construct
  source 1..1392: mol_type = other DNA
atggcactgccagtcacagcactgctgctgccactggcactgctgctccacgccgccagacccgacatcgtgctgacacagagcccagcaagcctggccgtgagcccaggccagagagccacaatcacatgcagagccagcgagtccgtgacaatcctgggctcccacctgatccactggtaccagcagaagcccggacagccacccaaactgctgatcaacctggccagcaacgtgaacacaggcgtgccagccagattcagcggcagcggcagcggaaccgacttcaccctgacaatcagctcagtggaaccagaagacaccgccaactactactgcctgcagagcagaacactgcccagaaccttcggccaaggcaccaaggtcgaaatcaagggctccacaagcggcagcggaaagcccggaagcggcgagggaagcaccaagggccagatccagctggtgcagagcggccccgaactgaagaaacccggcggaagcgtgaaaatctcctgcaaggccagcggctacaccttcaccgactacagcatcaactgggtgcgccaggccccaggcaagggcctggaatgggtcggctggatcaacaccgagacaagagagcccgcctacgcctacgacttcagaggcagattcaccttcagcgcagacacaagcaagagcatggcctacctgcagatcaactccctgagagcagaggacaccgccgtctactactgcgccctggactacacctacggcatggactactggggccagggcaccctcgtgaccgtgtccagcttcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaaccgcgtcaggtttagtagatccgctgacgcaccagcttatcagcagggccagaaccaactttataatgagctcaatcttggcaggagggaagagtacgatgtccttgaccgaaggcgaggtcgggacccagaaatgggcgggaggcctcaacgacgacgcaatccacaagagggactttacaacgaactgcagcgggatagaatggcagaggcttacagcgagataggaatgcgaggcgaaaggaggcgaggtagggggcacgatggtctctatcaaggcctgagtacggctacgagagacacatatgatgcgctgcatatgcaggccctccctcccaggtga

SEQ ID NO: 8 - DNA, 1392 bp, synthetic construct
  source 1..1392: mol_type = other DNA
atggcactgccagtcacagcactgctgctgccactggcactgctgctccacgccgccagacccgacatcgtgctgacacagagcccagcaagcctggccgtgagcccaggccagagagccacaatcacatgcagagccagcgagtccgtgacaatcctgggctcccacctgatccactggtaccagcagaagcccggacagccacccaaactgctgatcaacctggccagcaacgtgaacacaggcgtgccagccagattcagcggcagcggcagcggaaccgacttcaccctgacaatcagctcagtggaaccagaagacaccgccaactactactgcctgcagagcagaacactgcccagaaccttcggccaaggcaccaaggtcgaaatcaagggctccacaagcggcagcggaaagcccggaagcggcgagggaagcaccaagggccagatccagctggtgcagagcggccccgaactgaagaaacccggcggaagcgtgaaaatctcctgcaaggccagcggctacaccttcaccgactacagcatcaactgggtgcgccaggccccaggcaagggcctggaatgggtcggctggatcaacaccgagacaagagagcccgcctacgcctacgacttcagaggcagattcaccttcagcgcagacacaagcaagagcatggcctacctgcagatcaactccctgagagcagaggacaccgccgtctactactgcgccctggactacacctacggcatggactactggggccagggcaccctcgtgaccgtgtccagcttcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacagagtcgcattctcccggtccgctgacgcccctgcgtatcagcagggtcagaaccaactgtataatgagcttaatcttggaagaagagaggaatacgacgtattggatgcaaggcgagggcgcgacccggaaatgggcggagcgccccagaggcgagcaaatcctcaagagggactttataacgagctgcaggcggatgctatggcggaagcatacagtgaaataggaatggccggcgaacggcgcaggggcgcggggcatgatggtctgtatcagggtctttctactgcaacggccgacacgtatgacgccttgcatatgcaggcgctgccccccaggtga

SEQ ID NO: 9 - DNA, 1392 bp, synthetic construct
  source 1..1392: mol_type = other DNA
atggcactgccagtcacagcactgctgctgccactggcactgctgctccacgccgccagacccgacatcgtgctgacacagagcccagcaagcctggccgtgagcccaggccagagagccacaatcacatgcagagccagcgagtccgtgacaatcctgggctcccacctgatccactggtaccagcagaagcccggacagccacccaaactgctgatcaacctggccagcaacgtgaacacaggcgtgccagccagattcagcggcagcggcagcggaaccgacttcaccctgacaatcagctcagtggaaccagaagacaccgccaactactactgcctgcagagcagaacactgcccagaaccttcggccaaggcaccaaggtcgaaatcaagggctccacaagcggcagcggaaagcccggaagcggcgagggaagcaccaagggccagatccagctggtgcagagcggccccgaactgaagaaacccggcggaagcgtgaaaatctcctgcaaggccagcggctacaccttcaccgactacagcatcaactgggtgcgccaggccccaggcaagggcctggaatgggtcggctggatcaacaccgagacaagagagcccgcctacgcctacgacttcagaggcagattcaccttcagcgcagacacaagcaagagcatggcctacctgcagatcaactccctgagagcagaggacaccgccgtctactactgcgccctggactacacctacggcatggactactggggccagggcaccctcgtgaccgtgtccagcttcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacagagtcgacttctcccggtccgctgacgcccctgcgtatcagcagggtcagaaccaactgtataatgagcttaatcttggaagaagagaggaatacgacgtattggatgacaggcgagggcgcgacccggaaatgggcggagatccccagaggcgagacaatcctcaagagggactttataacgagctgcaggatgatgacatggcggaagcatacagtgaaataggaatggatggcgaacggcgcaggggcgacgggcatgatggtctgtatcagggtctttctactgcaacggacgacacgtatgacgccttgcatatgcaggcgctgccccccaggtga

SEQ ID NO: 10 - DNA, 1392 bp, synthetic construct
  source 1..1392: mol_type = other DNA
atggcactgccagtcacagcactgctgctgccactggcactgctgctccacgccgccagacccgacatcgtgctgacacagagcccagcaagcctggccgtgagcccaggccagagagccacaatcacatgcagagccagcgagtccgtgacaatcctgggctcccacctgatccactggtaccagcagaagcccggacagccacccaaactgctgatcaacctggccagcaacgtgaacacaggcgtgccagccagattcagcggcagcggcagcggaaccgacttcaccctgacaatcagctcagtggaaccagaagacaccgccaactactactgcctgcagagcagaacactgcccagaaccttcggccaaggcaccaaggtcgaaatcaagggctccacaagcggcagcggaaagcccggaagcggcgagggaagcaccaagggccagatccagctggtgcagagcggccccgaactgaagaaacccggcggaagcgtgaaaatctcctgcaaggccagcggctacaccttcaccgactacagcatcaactgggtgcgccaggccccaggcaagggcctggaatgggtcggctggatcaacaccgagacaagagagcccgcctacgcctacgacttcagaggcagattcaccttcagcgcagacacaagcaagagcatggcctacctgcagatcaactccctgagagcagaggacaccgccgtctactactgcgccctggactacacctacggcatggactactggggccagggcaccctcgtgaccgtgtccagcttcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacagagtcgaattctcccggtccgctgacgcccctgcgtatcagcagggtcagaaccaactgtataatgagcttaatcttggaagaagagaggaatacgacgtattggatgagaggcgagggcgcgacccggaaatgggcggagaaccccagaggcgagagaatcctcaagagggactttataacgagctgcaggaagatgagatggcggaagcatacagtgaaataggaatggaaggcgaacggcgcaggggcgaagggcatgatggtctgtatcagggtctttctactgcaacggaagacacgtatgacgccttgcatatgcaggcgctgccccccaggtga

SEQ ID NO: 11 - DNA, 46 bp, synthetic construct
  source 1..46: mol_type = other DNA
aggactcttctggtccccacagactcagagagaacccaccgccacc

SEQ ID NO: 12 - DNA, 286 bp, synthetic construct
  source 1..286: mol_type = other DNA
tgcccgtcctcaccaagactgactgcctgctgctttgctactgcccgggcccatgagactgacttcccactgctctgcctgcctctccccactgcactggcacagccccgccttgccgctgctgatccattgccggtgtgacccaagcacgcagcaatgcagctcaaaacgcttagcctagccacacccccacgggaaacagcagtgattaacctttagcaataaacgaaagtttaactaagctatactaaccccagggttggtcaatttcgtgccagccacacca

SEQ ID NO: 13 - RNA, 1724 nt, synthetic construct
  source 1..1724: mol_type = other RNA
aggactcttctggtccccacagactcagagagaacccaccgccaccatggcactgccagtcacagcactgctgctgccactggcactgctgctccacgccgccagacccgacatcgtgctgacacagagcccagcaagcctggccgtgagcccaggccagagagccacaatcacatgcagagccagcgagtccgtgacaatcctgggctcccacctgatccactggtaccagcagaagcccggacagccacccaaactgctgatcaacctggccagcaacgtgaacacaggcgtgccagccagattcagcggcagcggcagcggaaccgacttcaccctgacaatcagctcagtggaaccagaagacaccgccaactactactgcctgcagagcagaacactgcccagaaccttcggccaaggcaccaaggtcgaaatcaagggctccacaagcggcagcggaaagcccggaagcggcgagggaagcaccaagggccagatccagctggtgcagagcggccccgaactgaagaaacccggcggaagcgtgaaaatctcctgcaaggccagcggctacaccttcaccgactacagcatcaactgggtgcgccaggccccaggcaagggcctggaatgggtcggctggatcaacaccgagacaagagagcccgcctacgcctacgacttcagaggcagattcaccttcagcgcagacacaagcaagagcatggcctacctgcagatcaactccctgagagcagaggacaccgccgtctactactgcgccctggactacacctacggcatggactactggggccagggcaccctcgtgaccgtgtccagcttcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacagagtgaagttcagcagatccgccgacgcaccagcctaccagcagggacagaaccagctgtacaacgagctgaacctggggagaagagaagagtacgacgtgctggataagcggagaggcagagaccctgagatgggcggcaagccccaaagacggaagaacccacaagagggcctgtacaacgagctgcagaaagacaagatggccgaggcctacagcgagatcggaatgaagggcgagcgcagaagaggcaagggacacgacggactgtaccagggcctgagcacagccaccaaggatacctacgatgccctgcacatgcaggccctgccaccaagatgatgcccgtcctcaccaagactgactgcctgctgctttgctactgcccgggcccatgagactgacttcccactgctctgcctgcctctccccactgcactggcacagccccgccttgccgctgctgatccattgccggtgtgacccaagcacgcagcaatgcagctcaaaacgcttagcctagccacacccccacgggaaacagcagtgattaacctttagcaataaacgaaagtttaactaagctatactaaccccagggttggtcaatttcgtgccagccacacca

SEQ ID NO: 14 - RNA, 1724 nt, synthetic construct
  source 1..1724: mol_type = other RNA
aggactcttctggtccccacagactcagagagaacccaccgccaccatggcactgccagtcacagcactgctgctgccactggcactgctgctccacgccgccagacccgacatcgtgctgacacagagcccagcaagcctggccgtgagcccaggccagagagccacaatcacatgcagagccagcgagtccgtgacaatcctgggctcccacctgatccactggtaccagcagaagcccggacagccacccaaactgctgatcaacctggccagcaacgtgaacacaggcgtgccagccagattcagcggcagcggcagcggaaccgacttcaccctgacaatcagctcagtggaaccagaagacaccgccaactactactgcctgcagagcagaacactgcccagaaccttcggccaaggcaccaaggtcgaaatcaagggctccacaagcggcagcggaaagcccggaagcggcgagggaagcaccaagggccagatccagctggtgcagagcggccccgaactgaagaaacccggcggaagcgtgaaaatctcctgcaaggccagcggctacaccttcaccgactacagcatcaactgggtgcgccaggccccaggcaagggcctggaatgggtcggctggatcaacaccgagacaagagagcccgcctacgcctacgacttcagaggcagattcaccttcagcgcagacacaagcaagagcatggcctacctgcagatcaactccctgagagcagaggacaccgccgtctactactgcgccctggactacacctacggcatggactactggggccagggcaccctcgtgaccgtgtccagcttcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaaccgcgtcaggtttagtagatccgctgacgcaccagcttatcagcagggccagaaccaactttataatgagctcaatcttggcaggagggaagagtacgatgtccttgaccgaaggcgaggtcgggacccagaaatgggcgggaggcctcaacgacgacgcaatccacaagagggactttacaacgaactgcagcgggatagaatggcagaggcttacagcgagataggaatgcgaggcgaaaggaggcgaggtagggggcacgatggtctctatcaaggcctgagtacggctacgagagacacatatgatgcgctgcatatgcaggccctccctcccaggtgatgcccgtcctcaccaagactgactgcctgctgctttgctactgcccgggcccatgagactgacttcccactgctctgcctgcctctccccactgcactggcacagccccgccttgccgctgctgatccattgccggtgtgacccaagcacgcagcaatgcagctcaaaacgcttagcctagccacacccccacgggaaacagcagtgattaacctttagcaataaacgaaagtttaactaagctatactaaccccagggttggtcaatttcgtgccagccacacca

SEQ ID NO: 15 - RNA, 1724 nt, synthetic construct
  source 1..1724: mol_type = other RNA
aggactcttctggtccccacagactcagagagaacccaccgccaccatggcactgccagtcacagcactgctgctgccactggcactgctgctccacgccgccagacccgacatcgtgctgacacagagcccagcaagcctggccgtgagcccaggccagagagccacaatcacatgcagagccagcgagtccgtgacaatcctgggctcccacctgatccactggtaccagcagaagcccggacagccacccaaactgctgatcaacctggccagcaacgtgaacacaggcgtgccagccagattcagcggcagcggcagcggaaccgacttcaccctgacaatcagctcagtggaaccagaagacaccgccaactactactgcctgcagagcagaacactgcccagaaccttcggccaaggcaccaaggtcgaaatcaagggctccacaagcggcagcggaaagcccggaagcggcgagggaagcaccaagggccagatccagctggtgcagagcggccccgaactgaagaaacccggcggaagcgtgaaaatctcctgcaaggccagcggctacaccttcaccgactacagcatcaactgggtgcgccaggccccaggcaagggcctggaatgggtcggctggatcaacaccgagacaagagagcccgcctacgcctacgacttcagaggcagattcaccttcagcgcagacacaagcaagagcatggcctacctgcagatcaactccctgagagcagaggacaccgccgtctactactgcgccctggactacacctacggcatggactactggggccagggcaccctcgtgaccgtgtccagcttcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacagagtcgcattctcccggtccgctgacgcccctgcgtatcagcagggtcagaaccaactgtataatgagcttaatcttggaagaagagaggaatacgacgtattggatgcaaggcgagggcgcgacccggaaatgggcggagcgccccagaggcgagcaaatcctcaagagggactttataacgagctgcaggcggatgctatggcggaagcatacagtgaaataggaatggccggcgaacggcgcaggggcgcggggcatgatggtctgtatcagggtctttctactgcaacggccgacacgtatgacgccttgcatatgcaggcgctgccccccaggtgatgcccgtcctcaccaagactgactgcctgctgctttgctactgcccgggcccatgagactgacttcccactgctctgcctgcctctccccactgcactggcacagccccgccttgccgctgctgatccattgccggtgtgacccaagcacgcagcaatgcagctcaaaacgcttagcctagccacacccccacgggaaacagcagtgattaacctttagcaataaacgaaagtttaactaagctatactaaccccagggttggtcaatttcgtgccagccacacca

SEQ ID NO: 16 - RNA, 1724 nt, synthetic construct
  source 1..1724: mol_type = other RNA
aggactcttctggtccccacagactcagagagaacccaccgccaccatggcactgccagtcacagcactgctgctgccactggcactgctgctccacgccgccagacccgacatcgtgctgacacagagcccagcaagcctggccgtgagcccaggccagagagccacaatcacatgcagagccagcgagtccgtgacaatcctgggctcccacctgatccactggtaccagcagaagcccggacagccacccaaactgctgatcaacctggccagcaacgtgaacacaggcgtgccagccagattcagcggcagcggcagcggaaccgacttcaccctgacaatcagctcagtggaaccagaagacaccgccaactactactgcctgcagagcagaacactgcccagaaccttcggccaaggcaccaaggtcgaaatcaagggctccacaagcggcagcggaaagcccggaagcggcgagggaagcaccaagggccagatccagctggtgcagagcggccccgaactgaagaaacccggcggaagcgtgaaaatctcctgcaaggccagcggctacaccttcaccgactacagcatcaactgggtgcgccaggccccaggcaagggcctggaatgggtcggctggatcaacaccgagacaagagagcccgcctacgcctacgacttcagaggcagattcaccttcagcgcagacacaagcaagagcatggcctacctgcagatcaactccctgagagcagaggacaccgccgtctactactgcgccctggactacacctacggcatggactactggggccagggcaccctcgtgaccgtgtccagcttcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacagagtcgacttctcccggtccgctgacgcccctgcgtatcagcagggtcagaaccaactgtataatgagcttaatcttggaagaagagaggaatacgacgtattggatgacaggcgagggcgcgacccggaaatgggcggagatccccagaggcgagacaatcctcaagagggactttataacgagctgcaggatgatgacatggcggaagcatacagtgaaataggaatggatggcgaacggcgcaggggcgacgggcatgatggtctgtatcagggtctttctactgcaacggacgacacgtatgacgccttgcatatgcaggcgctgccccccaggtgatgcccgtcctcaccaagactgactgcctgctgctttgctactgcccgggcccatgagactgacttcccactgctctgcctgcctctccccactgcactggcacagccccgccttgccgctgctgatccattgccggtgtgacccaagcacgcagcaatgcagctcaaaacgcttagcctagccacacccccacgggaaacagcagtgattaacctttagcaataaacgaaagtttaactaagctatactaaccccagggttggtcaatttcgtgccagccacacca

SEQ ID NO: 17 - RNA, 1724 nt, synthetic construct
  source 1..1724: mol_type = other RNA
aggactcttctggtccccacagactcagagagaacccaccgccaccatggcactgccagtcacagcactgctgctgccactggcactgctgctccacgccgccagacccgacatcgtgctgacacagagcccagcaagcctggccgtgagcccaggccagagagccacaatcacatgcagagccagcgagtccgtgacaatcctgggctcccacctgatccactggtaccagcagaagcccggacagccacccaaactgctgatcaacctggccagcaacgtgaacacaggcgtgccagccagattcagcggcagcggcagcggaaccgacttcaccctgacaatcagctcagtggaaccagaagacaccgccaactactactgcctgcagagcagaacactgcccagaaccttcggccaaggcaccaaggtcgaaatcaagggctccacaagcggcagcggaaagcccggaagcggcgagggaagcaccaagggccagatccagctggtgcagagcggccccgaactgaagaaacccggcggaagcgtgaaaatctcctgcaaggccagcggctacaccttcaccgactacagcatcaactgggtgcgccaggccccaggcaagggcctggaatgggtcggctggatcaacaccgagacaagagagcccgcctacgcctacgacttcagaggcagattcaccttcagcgcagacacaagcaagagcatggcctacctgcagatcaactccctgagagcagaggacaccgccgtctactactgcgccctggactacacctacggcatggactactggggccagggcaccctcgtgaccgtgtccagcttcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacagagtcgaattctcccggtccgctgacgcccctgcgtatcagcagggtcagaaccaactgtataatgagcttaatcttggaagaagagaggaatacgacgtattggatgagaggcgagggcgcgacccggaaatgggcggagaaccccagaggcgagagaatcctcaagagggactttataacgagctgcaggaagatgagatggcggaagcatacagtgaaataggaatggaaggcgaacggcgcaggggcgaagggcatgatggtctgtatcagggtctttctactgcaacggaagacacgtatgacgccttgcatatgcaggcgctgccccccaggtgatgcccgtcctcaccaagactgactgcctgctgctttgctactgcccgggcccatgagactgacttcccactgctctgcctgcctctccccactgcactggcacagccccgccttgccgctgctgatccattgccggtgtgacccaagcacgcagcaatgcagctcaaaacgcttagcctagccacacccccacgggaaacagcagtgattaacctttagcaataaacgaaagtttaactaagctatactaaccccagggttggtcaatttcgtgccagccacacca

SEQ ID NO: 18 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 19 - protein, 113 aa, synthetic construct
RVAFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 20 - protein, 113 aa, synthetic construct
RVKFSRSADAPAYQQGQNQLYNELNLGRREEYDVLDARRGRDPEMGGKPQRRKNPQEGLYNELQKDKMAEAYSEIGMKGERRRGKGHDGLYQGLSTATKDTYDALHMQALPPR

SEQ ID NO: 21 - protein, 113 aa, synthetic construct
RVKFSRSADAPAYQQGQNQLYNELNLGRREEYDVLDKRRGRDPEMGGAPQRRKNPQEGLYNELQKDKMAEAYSEIGMKGERRRGKGHDGLYQGLSTATKDTYDALHMQALPPR

SEQ ID NO: 22 - protein, 113 aa, synthetic construct
RVKFSRSADAPAYQQGQNQLYNELNLGRREEYDVLDKRRGRDPEMGGKPQRRANPQEGLYNELQKDKMAEAYSEIGMKGERRRGKGHDGLYQGLSTATKDTYDALHMQALPPR

SEQ ID NO: 23 - protein, 113 aa, synthetic construct
RVKFSRSADAPAYQQGQNQLYNELNLGRREEYDVLDKRRGRDPEMGGKPQRRKNPQEGLYNELQADKMAEAYSEIGMKGERRRGKGHDGLYQGLSTATKDTYDALHMQALPPR

SEQ ID NO: 24 - protein, 113 aa, synthetic construct
RVKFSRSADAPAYQQGQNQLYNELNLGRREEYDVLDKRRGRDPEMGGKPQRRKNPQEGLYNELQKDAMAEAYSEIGMKGERRRGKGHDGLYQGLSTATKDTYDALHMQALPPR

SEQ ID NO: 25 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMAGERRRGKGHDGLYQGLSTATKDTYDALHMQALPPR

SEQ ID NO: 26 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGAGHDGLYQGLSTATKDTYDALHMQALPPR

SEQ ID NO: 27 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATADTYDALHMQALPPR

SEQ ID NO: 28 - protein, 113 aa, synthetic construct
RVKFSRSADAPAYQQGQNQLYNELNLGRREEYDVLDARRGRDPEMGGKPQRRANPQEGLYNELQADAMAEAYSEIGMAGERRRGAGHDGLYQGLSTATADTYDALHMQALPPR

SEQ ID NO: 29 - protein, 113 aa, synthetic construct
RVKFSRSADAPAYQQGQNQLYNELNLGRREEYDVLDARRGRDPEMGGKPQRRKNPQEGLYNELQADAMAEAYSEIGMAGERRRGAGHDGLYQGLSTATADTYDALHMQALPPR

SEQ ID NO: 30 - protein, 113 aa, synthetic construct
RVAFSRSADAPAYQQGQNQLYNELNLGRREEYDVLDKRRGRDPEMGGAPQRRKNPQEGLYNELQKDKMAEAYSEIGMKGERRRGKGHDGLYQGLSTATKDTYDALHMQALPPR

SEQ ID NO: 31 - protein, 113 aa, synthetic construct
RVAFSRSADAPAYQQGQNQLYNELNLGRREEYDVLDARRGRDPEMGGAPQRRANPQEGLYNELQKDKMAEAYSEIGMAGERRRGAGHDGLYQGLSTATADTYDALHMQALPPR

SEQ ID NO: 32 - protein, 113 aa, synthetic construct
RVAFSRSADAPAYQQGQNQLYNELNLGRREEYDVLDARRGRDPEMGGAPQRRANPQEGLYNELQADAMAEAYSEIGMKGERRRGKGHDGLYQGLSTATADTYDALHMQALPPR

SEQ ID NO: 33 - protein, 113 aa, synthetic construct
RVAFSRSADAPAYQQGQNQLYNELNLGRREEYDVLDKRRGRDPEMGGAPQRRANPQEGLYNELQADAMAEAYSEIGMAGERRRGAGHDGLYQGLSTATKDTYDALHMQALPPR

SEQ ID NO: 34 - protein, 113 aa, synthetic construct
RVKFSRSADAPAYQQGQNQLYNELNLGRREEYDVLDARRGRDPEMGGAPQRRANPQEGLYNELQADAMAEAYSEIGMAGERRRGAGHDGLYQGLSTATADTYDALHMQALPPR

SEQ ID NO: 35 - protein, 113 aa, synthetic construct
RVAFSRSADAPAYQQGQNQLYNELNLGRREEYDVLDARRGRDPEMGGKPQRRANPQEGLYNELQADAMAEAYSEIGMAGERRRGAGHDGLYQGLSTATADTYDALHMQALPPR

SEQ ID NO: 36 - protein, 113 aa, synthetic construct
RVAFSRSADAPAYQQGQNQLYNELNLGRREEYDVLDARRGRDPEMGGAPQRRANPQEGLYNELQADKMAEAYSEIGMAGERRRGAGHDGLYQGLSTATADTYDALHMQALPPR

SEQ ID NO: 37 - protein, 113 aa, synthetic construct
RVAFSRSADAPAYQQGQNQLYNELNLGRREEYDVLDARRGRDPEMGGAPQRRANPQEGLYNELQADAMAEAYSEIGMAGERRRGKGHDGLYQGLSTATADTYDALHMQALPPR

SEQ ID NO: 38 - protein, 113 aa, synthetic construct
RVAFSRSADAPAYQQGQNQLYNELNLGRREEYDVLDKRRGRDPEMGGAPQRRANPQEGLYNELQADAMAEAYSEIGMAGERRRGAGHDGLYQGLSTATADTYDALHMQALPPR

SEQ ID NO: 39 - protein, 113 aa, synthetic construct
RVAFSRSADAPAYQQGQNQLYNELNLGRREEYDVLDARRGRDPEMGGAPQRRKNPQEGLYNELQADAMAEAYSEIGMAGERRRGAGHDGLYQGLSTATADTYDALHMQALPPR

SEQ ID NO: 40 - protein, 113 aa, synthetic construct
RVAFSRSADAPAYQQGQNQLYNELNLGRREEYDVLDARRGRDPEMGGAPQRRANPQEGLYNELQKDAMAEAYSEIGMAGERRRGAGHDGLYQGLSTATADTYDALHMQALPPR

SEQ ID NO: 41 - protein, 113 aa, synthetic construct
RVAFSRSADAPAYQQGQNQLYNELNLGRREEYDVLDARRGRDPEMGGAPQRRANPQEGLYNELQADAMAEAYSEIGMKGERRRGAGHDGLYQGLSTATADTYDALHMQALPPR

SEQ ID NO: 42 - protein, 113 aa, synthetic construct
RVAFSRSADAPAYQQGQNQLYNELNLGRREEYDVLDARRGRDPEMGGAPQRRANPQEGLYNELQADAMAEAYSEIGMAGERRRGAGHDGLYQGLSTATKDTYDALHMQALPPR

SEQ ID NO: 43 - protein, 113 aa, synthetic construct
RVAFSRSADAPAYQQGQNQLYNELNLGRREEYDVLDARRGRDPEMGGAPQRRANPQEGLYNELQADAMAEAYSEIGMAGERRRGAGHDGLYQGLSTATADTYDALHMQALPPR

SEQ ID NO: 44 - protein, 113 aa, synthetic construct
RVVFSRSADAPAYQQGQNQLYNELNLGRREEYDVLDVRRGRDPEMGGVPQRRVNPQEGLYNELQVDVMAEAYSEIGMVGERRRGVGHDGLYQGLSTATVDTYDALHMQALPPR

SEQ ID NO: 45 - protein, 113 aa, synthetic construct
RVIFSRSADAPAYQQGQNQLYNELNLGRREEYDVLDIRRGRDPEMGGIPQRRINPQEGLYNELQIDIMAEAYSEIGMIGERRRGIGHDGLYQGLSTATIDTYDALHMQALPPR

SEQ ID NO: 46 - protein, 113 aa, synthetic construct
RVLFSRSADAPAYQQGQNQLYNELNLGRREEYDVLDLRRGRDPEMGGLPQRRLNPQEGLYNELQLDLMAEAYSEIGMLGERRRGLGHDGLYQGLSTATLDTYDALHMQALPPR

SEQ ID NO: 47 - protein, 113 aa, synthetic construct
RVMFSRSADAPAYQQGQNQLYNELNLGRREEYDVLDMRRGRDPEMGGMPQRRMNPQEGLYNELQMDMMAEAYSEIGMMGERRRGMGHDGLYQGLSTATMDTYDALHMQALPPR

SEQ ID NO: 48 - protein, 113 aa, synthetic construct
RVFFSRSADAPAYQQGQNQLYNELNLGRREEYDVLDFRRGRDPEMGGFPQRRFNPQEGLYNELQFDFMAEAYSEIGMFGERRRGFGHDGLYQGLSTATFDTYDALHMQALPPR

SEQ ID NO: 49 - protein, 113 aa, synthetic construct
RVYFSRSADAPAYQQGQNQLYNELNLGRREEYDVLDYRRGRDPEMGGYPQRRYNPQEGLYNELQYDYMAEAYSEIGMYGERRRGYGHDGLYQGLSTATYDTYDALHMQALPPR

SEQ ID NO: 50 - protein, 113 aa, synthetic construct
RVSFSRSADAPAYQQGQNQLYNELNLGRREEYDVLDSRRGRDPEMGGSPQRRSNPQEGLYNELQSDSMAEAYSEIGMSGERRRGSGHDGLYQGLSTATSDTYDALHMQALPPR

SEQ ID NO: 51 - protein, 113 aa, synthetic construct
RVTFSRSADAPAYQQGQNQLYNELNLGRREEYDVLDTRRGRDPEMGGTPQRRTNPQEGLYNELQTDTMAEAYSEIGMTGERRRGTGHDGLYQGLSTATTDTYDALHMQALPPR

SEQ ID NO: 52 - protein, 113 aa, synthetic construct
RVNFSRSADAPAYQQGQNQLYNELNLGRREEYDVLDNRRGRDPEMGGNPQRRNNPQEGLYNELQNDNMAEAYSEIGMNGERRRGNGHDGLYQGLSTATNDTYDALHMQALPPR

SEQ ID NO: 53 - protein, 113 aa, synthetic construct
RVQFSRSADAPAYQQGQNQLYNELNLGRREEYDVLDQRRGRDPEMGGQPQRRQNPQEGLYNELQQDQMAEAYSEIGMQGERRRGQGHDGLYQGLSTATQDTYDALHMQALPPR

SEQ ID NO: 54 - protein, 113 aa, synthetic construct
RVHFSRSADAPAYQQGQNQLYNELNLGRREEYDVLDHRRGRDPEMGGHPQRRHNPQEGLYNELQHDHMAEAYSEIGMHGERRRGHGHDGLYQGLSTATHDTYDALHMQALPPR

SEQ ID NO: 55 - protein, 113 aa, synthetic construct
RVDFSRSADAPAYQQGQNQLYNELNLGRREEYDVLDDRRGRDPEMGGDPQRRDNPQEGLYNELQDDDMAEAYSEIGMDGERRRGDGHDGLYQGLSTATDDTYDALHMQALPPR

SEQ ID NO: 56 - protein, 113 aa, synthetic construct
RVEFSRSADAPAYQQGQNQLYNELNLGRREEYDVLDERRGRDPEMGGEPQRRENPQEGLYNELQEDEMAEAYSEIGMEGERRRGEGHDGLYQGLSTATEDTYDALHMQALPPR

SEQ ID NO: 57 - protein, 154 aa, synthetic construct
RSKRSRLLHSDYMNMTPRRPGPTRKHYQPYAPPRDFAAYRSRVAFSRSADAPAYQQGQNQLYNELNLGRREEYDVLDARRGRDPEMGGAPQRRANPQEGLYNELQADAMAEAYSEIGMAGERRRGAGHDGLYQGLSTATADTYDALHMQALPPR

SEQ ID NO: 58 - protein, 154 aa, synthetic construct
RSARSRLLHSDYMNMTPRRPGPTRAHYQPYAPPRDFAAYRSRVAFSRSADAPAYQQGQNQLYNELNLGRREEYDVLDARRGRDPEMGGAPQRRANPQEGLYNELQADAMAEAYSEIGMAGERRRGAGHDGLYQGLSTATADTYDALHMQALPPR

SEQ ID NO: 59 - protein, 155 aa, synthetic construct
KRGRKKLLYIFKQPFMRPVQTTQEEDGCSCRFPEEEEGGCELRVAFSRSADAPAYQQGQNQLYNELNLGRREEYDVLDARRGRDPEMGGAPQRRANPQEGLYNELQADAMAEAYSEIGMAGERRRGAGHDGLYQGLSTATADTYDALHMQALPPR

SEQ ID NO: 60 - protein, 155 aa, synthetic construct
ARGRAALLYIFAQPFMRPVQTTQEEDGCSCRFPEEEEGGCELRVAFSRSADAPAYQQGQNQLYNELNLGRREEYDVLDARRGRDPEMGGAPQRRANPQEGLYNELQADAMAEAYSEIGMAGERRRGAGHDGLYQGLSTATADTYDALHMQALPPR

SEQ ID NO: 61 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 154 aa, synthetic construct
RSERSRLLHSDYMNMTPRRPGPTREHYQPYAPPRDFAAYRSRVAFSRSADAPAYQQGQNQLYNELNLGRREEYDVLDARRGRDPEMGGAPQRRANPQEGLYNELQADAMAEAYSEIGMAGERRRGAGHDGLYQGLSTATADTYDALHMQALPPR

SEQ ID NO: 63 - protein, 154 aa, synthetic construct
RSKRSRLLHSDYMNMTPRRPGPTRKHYQPYAPPRDFAAYRSRVEFSRSADAPAYQQGQNQLYNELNLGRREEYDVLDERRGRDPEMGGEPQRRENPQEGLYNELQEDEMAEAYSEIGMEGERRRGEGHDGLYQGLSTATEDTYDALHMQALPPR

SEQ ID NO: 64 - protein, 154 aa, synthetic construct
RSARSRLLHSDYMNMTPRRPGPTRAHYQPYAPPRDFAAYRSRVEFSRSADAPAYQQGQNQLYNELNLGRREEYDVLDERRGRDPEMGGEPQRRENPQEGLYNELQEDEMAEAYSEIGMEGERRRGEGHDGLYQGLSTATEDTYDALHMQALPPR

SEQ ID NO: 65 - protein, 154 aa, synthetic construct
RSERSRLLHSDYMNMTPRRPGPTREHYQPYAPPRDFAAYRSRVEFSRSADAPAYQQGQNQLYNELNLGRREEYDVLDERRGRDPEMGGEPQRRENPQEGLYNELQEDEMAEAYSEIGMEGERRRGEGHDGLYQGLSTATEDTYDALHMQALPPR

SEQ ID NO: 66 - protein, 153 aa, synthetic construct
RSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - RNA, 1847 nt, synthetic construct
  source 1..1847: mol_type = other RNA
aggactcttctggtccccacagactcagagagaacccaccgccaccatggcactgccagtcacagcactgctgctgccactggcactgctgctccacgccgccagacccgacatcgtgctgacacagagcccagcaagcctggccgtgagcccaggccagagagccacaatcacatgcagagccagcgagtccgtgacaatcctgggctcccacctgatccactggtaccagcagaagcccggacagccacccaaactgctgatcaacctggccagcaacgtgaacacaggcgtgccagccagattcagcggcagcggcagcggaaccgacttcaccctgacaatcagctcagtggaaccagaagacaccgccaactactactgcctgcagagcagaacactgcccagaaccttcggccaaggcaccaaggtcgaaatcaagggctccacaagcggcagcggaaagcccggaagcggcgagggaagcaccaagggccagatccagctggtgcagagcggccccgaactgaagaaacccggcggaagcgtgaaaatctcctgcaaggccagcggctacaccttcaccgactacagcatcaactgggtgcgccaggccccaggcaagggcctggaatgggtcggctggatcaacaccgagacaagagagcccgcctacgcctacgacttcagaggcagattcaccttcagcgcagacacaagcaagagcatggcctacctgcagatcaactccctgagagcagaggacaccgccgtctactactgcgccctggactacacctacggcatggactactggggccagggcaccctcgtgaccgtgtccagcttcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacaggtccgagagatctcgccttctccattctgactacatgaacatgacgccccgcaggccagggccaacacgagagcattatcagccatatgccccaccacgcgattttgctgcgtaccgctctagagtcgaattctcccggtccgctgacgcccctgcgtatcagcagggtcagaaccaactgtataatgagcttaatcttggaagaagagaggaatacgacgtattggatgagaggcgagggcgcgacccggaaatgggcggagaaccccagaggcgagagaatcctcaagagggactttataacgagctgcaggaagatgagatggcggaagcatacagtgaaataggaatggaaggcgaacggcgcaggggcgaagggcatgatggtctgtatcagggtctttctactgcaacggaagacacgtatgacgccttgcatatgcaggcgctgccccccaggtgatgcccgtcctcaccaagactgactgcctgctgctttgctactgcccgggcccatgagactgacttcccactgctctgcctgcctctccccactgcactggcacagccccgccttgccgctgctgatccattgccggtgtgacccaagcacgcagcaatgcagctcaaaacgcttagcctagccacacccccacgggaaacagcagtgattaacctttagcaataaacgaaagtttaactaagctatactaaccccagggttggtcaatttcgtgccagccacacca

SEQ ID NO: 68 - protein, 504 aa, synthetic construct
MALPVTALLLPLALLLHAARPDIVLTQSPASLAVSPGQRATITCRASESVTILGSHLIHWYQQKPGQPPKLLINLASNVNTGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTDYSINWVRQAPGKGLEWVGWINTETREPAYAYDFRGRFTFSADTSKSMAYLQINSLRAEDTAVYYCALDYTYGMDYWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSERSRLLHSDYMNMTPRRPGPTREHYQPYAPPRDFAAYRSRVEFSRSADAPAYQQGQNQLYNELNLGRREEYDVLDERRGRDPEMGGEPQRRENPQEGLYNELQEDEMAEAYSEIGMEGERRRGEGHDGLYQGLSTATEDTYDALHMQALPPR

SEQ ID NO: 69 - protein, 113 aa, synthetic construct
RVAFSRSADAPAYQQGQNQLYNELNLGRREEYDVLDERRGRDPEMGGEPQRRENPQEGLYNELQEDEMAEAYSEIGMEGERRRGEGHDGLYQGLSTATEDTYDALHMQALPPR

SEQ ID NO: 70 - protein, 113 aa, synthetic construct
RVEFSRSADAPAYQQGQNQLYNELNLGRREEYDVLDARRGRDPEMGGEPQRRENPQEGLYNELQEDEMAEAYSEIGMEGERRRGEGHDGLYQGLSTATEDTYDALHMQALPPR

SEQ ID NO: 71 - protein, 113 aa, synthetic construct
RVEFSRSADAPAYQQGQNQLYNELNLGRREEYDVLDERRGRDPEMGGAPQRRENPQEGLYNELQEDEMAEAYSEIGMEGERRRGEGHDGLYQGLSTATEDTYDALHMQALPPR

SEQ ID NO: 72 - protein, 113 aa, synthetic construct
RVEFSRSADAPAYQQGQNQLYNELNLGRREEYDVLDERRGRDPEMGGEPQRRANPQEGLYNELQEDEMAEAYSEIGMEGERRRGEGHDGLYQGLSTATEDTYDALHMQALPPR

SEQ ID NO: 73 - protein, 113 aa, synthetic construct
RVEFSRSADAPAYQQGQNQLYNELNLGRREEYDVLDERRGRDPEMGGEPQRRENPQEGLYNELQADEMAEAYSEIGMEGERRRGEGHDGLYQGLSTATEDTYDALHMQALPPR

SEQ ID NO: 74 - protein, 113 aa, synthetic construct
RVEFSRSADAPAYQQGQNQLYNELNLGRREEYDVLDERRGRDPEMGGEPQRRENPQEGLYNELQEDAMAEAYSEIGMEGERRRGEGHDGLYQGLSTATEDTYDALHMQALPPR

SEQ ID NO: 75 - protein, 113 aa, synthetic construct
RVEFSRSADAPAYQQGQNQLYNELNLGRREEYDVLDERRGRDPEMGGEPQRRENPQEGLYNELQEDEMAEAYSEIGMAGERRRGEGHDGLYQGLSTATEDTYDALHMQALPPR

SEQ ID NO: 76 - protein, 113 aa, synthetic construct
RVEFSRSADAPAYQQGQNQLYNELNLGRREEYDVLDERRGRDPEMGGEPQRRENPQEGLYNELQEDEMAEAYSEIGMEGERRRGAGHDGLYQGLSTATEDTYDALHMQALPPR

SEQ ID NO: 77 - protein, 113 aa, synthetic construct
RVEFSRSADAPAYQQGQNQLYNELNLGRREEYDVLDERRGRDPEMGGEPQRRENPQEGLYNELQEDEMAEAYSEIGMEGERRRGEGHDGLYQGLSTATADTYDALHMQALPPR

SEQ ID NO: 78 - protein, 113 aa, synthetic construct
RVEFSRSADAPAYQQGQNQLYNELNLGRREEYDVLDARRGRDPEMGGAPQRRANPQEGLYNELQADAMAEAYSEIGMAGERRRGAGHDGLYQGLSTATADTYDALHMQALPPR

SEQ ID NO: 79 - protein, 113 aa, synthetic construct
RVAFSRSADAPAYQQGQNQLYNELNLGRREEYDVLDERRGRDPEMGGAPQRRANPQEGLYNELQADAMAEAYSEIGMAGERRRGAGHDGLYQGLSTATADTYDALHMQALPPR

SEQ ID NO: 80 - protein, 113 aa, synthetic construct
RVAFSRSADAPAYQQGQNQLYNELNLGRREEYDVLDARRGRDPEMGGEPQRRANPQEGLYNELQADAMAEAYSEIGMAGERRRGAGHDGLYQGLSTATADTYDALHMQALPPR

SEQ ID NO: 81 - protein, 113 aa, synthetic construct
RVAFSRSADAPAYQQGQNQLYNELNLGRREEYDVLDARRGRDPEMGGAPQRRENPQEGLYNELQADAMAEAYSEIGMAGERRRGAGHDGLYQGLSTATADTYDALHMQALPPR

SEQ ID NO: 82 - protein, 113 aa, synthetic construct
RVAFSRSADAPAYQQGQNQLYNELNLGRREEYDVLDARRGRDPEMGGAPQRRANPQEGLYNELQEDAMAEAYSEIGMAGERRRGAGHDGLYQGLSTATADTYDALHMQALPPR

SEQ ID NO: 83 - protein, 113 aa, synthetic construct
RVAFSRSADAPAYQQGQNQLYNELNLGRREEYDVLDARRGRDPEMGGAPQRRANPQEGLYNELQADEMAEAYSEIGMAGERRRGAGHDGLYQGLSTATADTYDALHMQALPPR

SEQ ID NO: 84 - protein, 113 aa, synthetic construct
RVAFSRSADAPAYQQGQNQLYNELNLGRREEYDVLDARRGRDPEMGGAPQRRANPQEGLYNELQADAMAEAYSEIGMEGERRRGAGHDGLYQGLSTATADTYDALHMQALPPR

SEQ ID NO: 85 - protein, 113 aa, synthetic construct
RVAFSRSADAPAYQQGQNQLYNELNLGRREEYDVLDARRGRDPEMGGAPQRRANPQEGLYNELQADAMAEAYSEIGMAGERRRGEGHDGLYQGLSTATADTYDALHMQALPPR

SEQ ID NO: 86 - protein, 113 aa, synthetic construct
RVAFSRSADAPAYQQGQNQLYNELNLGRREEYDVLDARRGRDPEMGGAPQRRANPQEGLYNELQADAMAEAYSEIGMAGERRRGAGHDGLYQGLSTATEDTYDALHMQALPPR

SEQ ID NO: 87 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 88 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYAGGSYAMDYWGQGTSVTVSS

SEQ ID NO: 89 - RNA, 1874 nt, synthetic construct
  source 1..1874: mol_type = other RNA
aggactcttctggtccccacagactcagagagaacccaccgccaccatggcactgccagtcacagcactgctgctgccactggcactgctgctccacgccgccagacccgagcagaagctgatcagcgaggaggacctggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggctccacaagcggcagcggaaagcccggaagcggcgagggaagcaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcattcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacaggtccgagagatctcgccttctccattctgactacatgaacatgacgccccgcaggccagggccaacacgagagcattatcagccatatgccccaccacgcgattttgctgcgtaccgctctagagtcgaattctcccggtccgctgacgcccctgcgtatcagcagggtcagaaccaactgtataatgagcttaatcttggaagaagagaggaatacgacgtattggatgagaggcgagggcgcgacccggaaatgggcggagaaccccagaggcgagagaatcctcaagagggactttataacgagctgcaggaagatgagatggcggaagcatacagtgaaataggaatggaaggcgaacggcgcaggggcgaagggcatgatggtctgtatcagggtctttctactgcaacggaagacacgtatgacgccttgcatatgcaggcgctgccccccaggtgatgcccgtcctcaccaagactgactgcctgctgctttgctactgcccgggcccatgagactgacttcccactgctctgcctgcctctccccactgcactggcacagccccgccttgccgctgctgatccattgccggtgtgacccaagcacgcagcaatgcagctcaaaacgcttagcctagccacacccccacgggaaacagcagtgattaacctttagcaataaacgaaagtttaactaagctatactaaccccagggttggtcaatttcgtgccagccacacca

SEQ ID NO: 90 - RNA, 1874 nt, synthetic construct
  source 1..1874: mol_type = other RNA
aggactcttctggtccccacagactcagagagaacccaccgccaccatggcactgccagtcacagcactgctgctgccactggcactgctgctccacgccgccagacccgagcagaagctgatcagcgaggaggacctggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggctccacaagcggcagcggaaagcccggaagcggcgagggaagcaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattacgcaggtggtagctatgctatggactactggggccaaggaacctcagtcaccgtctcctcattcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacaggtccgagagatctcgccttctccattctgactacatgaacatgacgccccgcaggccagggccaacacgagagcattatcagccatatgccccaccacgcgattttgctgcgtaccgctctagagtcgaattctcccggtccgctgacgcccctgcgtatcagcagggtcagaaccaactgtataatgagcttaatcttggaagaagagaggaatacgacgtattggatgagaggcgagggcgcgacccggaaatgggcggagaaccccagaggcgagagaatcctcaagagggactttataacgagctgcaggaagatgagatggcggaagcatacagtgaaataggaatggaaggcgaacggcgcaggggcgaagggcatgatggtctgtatcagggtctttctactgcaacggaagacacgtatgacgccttgcatatgcaggcgctgccccccaggtgatgcccgtcctcaccaagactgactgcctgctgctttgctactgcccgggcccatgagactgacttcccactgctctgcctgcctctccccactgcactggcacagccccgccttgccgctgctgatccattgccggtgtgacccaagcacgcagcaatgcagctcaaaacgcttagcctagccacacccccacgggaaacagcagtgattaacctttagcaataaacgaaagtttaactaagctatactaaccccagggttggtcaatttcgtgccagccacacca

SEQ ID NO: 91 - RNA, 1874 nt, synthetic construct
  source 1..1874: mol_type = other RNA
aggactcttctggtccccacagactcagagagaacccaccgccaccatggcactgccagtcacagcactgctgctgccactggcactgctgctccacgccgccagacccgagcagaaactcatctcagaagaggatctggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggctccacaagcggcagcggaaagcccggaagcggcgagggaagcaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcattcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacaggagcaagaggtccaggctgctgcatagcgactacatgaacatgacccccagaaggcccggacccaccaggaagcactaccagccctacgccccccctagggattttgctgcctacaggtccagagtgaagttcagcagatccgccgacgcaccagcctaccagcagggacagaaccagctgtacaacgagctgaacctggggagaagagaagagtacgacgtgctggataagcggagaggcagagaccctgagatgggcggcaagccccaaagacggaagaacccacaagagggcctgtacaacgagctgcagaaagacaagatggccgaggcctacagcgagatcggaatgaagggcgagcgcagaagaggcaagggacacgacggactgtaccagggcctgagcacagccaccaaggatacctacgatgccctgcacatgcaggccctgccaccaagatgatgcccgtcctcaccaagactgactgcctgctgctttgctactgcccgggcccatgagactgacttcccactgctctgcctgcctctccccactgcactggcacagccccgccttgccgctgctgatccattgccggtgtgacccaagcacgcagcaatgcagctcaaaacgcttagcctagccacacccccacgggaaacagcagtgattaacctttagcaataaacgaaagtttaactaagctatactaaccccagggttggtcaatttcgtgccagccacacca

SEQ ID NO: 92 - RNA, 1874 nt, synthetic construct
  source 1..1874: mol_type = other RNA
aggactcttctggtccccacagactcagagagaacccaccgccaccatggcactgccagtcacagcactgctgctgccactggcactgctgctccacgccgccagacccgagcagaaactcatctcagaagaggatctggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggctccacaagcggcagcggaaagcccggaagcggcgagggaagcaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattacgcaggtggtagctatgctatggactactggggccaaggaacctcagtcaccgtctcctcattcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacaggagcaagaggtccaggctgctgcatagcgactacatgaacatgacccccagaaggcccggacccaccaggaagcactaccagccctacgccccccctagggattttgctgcctacaggtccagagtgaagttcagcagatccgccgacgcaccagcctaccagcagggacagaaccagctgtacaacgagctgaacctggggagaagagaagagtacgacgtgctggataagcggagaggcagagaccctgagatgggcggcaagccccaaagacggaagaacccacaagagggcctgtacaacgagctgcagaaagacaagatggccgaggcctacagcgagatcggaatgaagggcgagcgcagaagaggcaagggacacgacggactgtaccagggcctgagcacagccaccaaggatacctacgatgccctgcacatgcaggccctgccaccaagatgatgcccgtcctcaccaagactgactgcctgctgctttgctactgcccgggcccatgagactgacttcccactgctctgcctgcctctccccactgcactggcacagccccgccttgccgctgctgatccattgccggtgtgacccaagcacgcagcaatgcagctcaaaacgcttagcctagccacacccccacgggaaacagcagtgattaacctttagcaataaacgaaagtttaactaagctatactaaccccagggttggtcaatttcgtgccagccacacca

SEQ ID NO: 93 - protein, 503 aa, synthetic construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - protein, 464 aa, synthetic construct
MALPVTALLLPLALLLHAARPDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKGSTSGSGKPGSGEGSTKGQVQLVESGGGVVQPGRSLRLSCAASGFAFSRYGMHWVRQAPGKGLEWVAVIWYDGSNKYYADSVKGRFTISRDNSKNTQYLQMNSLRAEDTAVYYCARGGDFLYYYYYGMDVWGQGTTVTVSSFVPVFLPAKPTTTPAPRPPTPAPTIASQPLSLRPEACRPAAGGAVHTRGLDFACDIYIWAPLAGTCGVLLLSLVITLYCNHRNRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 95 - RNA, 1847 nt, synthetic construct
  source 1..1847: mol_type = other RNA
aggactcttctggtccccacagactcagagagaacccaccgccgccaccatggccctgcccgtgacagctctgctgctccctctggctctgctgctgcacgctgccagacccgacatcgtgctgacacaaagcccccctagcctggccatgagcctgggcaagagggccacaatctcctgcagggccagcgagtccgtgaccatcctgggcagccacctgatccactggtaccagcagaagcctggccagccccctaccctgctgatccagctggcctccaacgtgcagacaggagtgcccgctaggttctccggctccggcagcagaaccgactttaccctgaccatcgaccccgtcgaggaggacgacgtggctgtctactattgcctgcagagcaggaccatccccaggaccttcggaggcggcacaaagctggagatcaagggcagcaccagcggctccggaaagcctggcagcggagaaggctccaccaagggacagattcagctggtgcagagcggacccgagctgaagaagcccggcgaaacagtcaagatcagctgcaaagcttccggctacaccttcacagactacagcatcaactgggtgaaaagggcccccggcaagggcctgaaatggatgggctggatcaacaccgagaccagggagcccgcctacgcctacgactttaggggcaggttcgccttcagcctggagacatccgctagcaccgcttacctgcaaattaataacctgaagtacgaggacaccgccacctatttttgtgccctggactacagctacgcaatggactattggggacagggaacctccgtgaccgtgagcagctttgtgcccgtgttcctgcccgccaaacccaccaccacacctgctcctaggccccctacacctgcccctaccatcgcttcccagcccctgagcctcagacctgaagcctgcagacctgctgctggaggcgccgtccacacaaggggactggacttcgcctgtgacatctacatctgggctcctctcgccggcacctgtggagtgctgctgctgtccctggtcatcaccctgtactgcaaccacaggaacaggagcaagaggtccaggctgctgcatagcgactacatgaacatgacccccagaaggcccggacccaccaggaagcactaccagccctacgccccccctagggattttgctgcctacaggtccagagtgaagttcagcagatccgctgatgcccctgcctatcagcagggccagaaccaactctacaacgagctgaacctgggaaggagggaggagtacgacgtgctcgacaagagaaggggaagggatcctgaaatgggcggcaagcccaggagaaagaaccctcaggagggcctgtacaacgaactgcagaaggataagatggccgaggcctacagcgaaatcggcatgaaaggcgagagaaggagaggcaagggccacgacggactgtaccagggcctgtccaccgccaccaaagacacctacgatgccctgcacatgcaggccctgccccccaggtgatgcccgtcctcaccaagactgactgcctgctgctttgctactgcccgggcccatgagactgacttcccactgctctgcctgcctctccccactgcactggcacagccccgccttgccgctgctgatccattgccggtgtgacccaagcacgcagcaatgcagctcaaaacgcttagcctagccacacccccacgggaaacagcagtgattaacctttagcaataaacgaaagtttaactaagctatactaaccccagggttggtcaatttcgtgccagccacacca

SEQ ID NO: 96 - RNA, 1724 nt, synthetic construct
  source 1..1724: mol_type = other RNA
aggactcttctggtccccacagactcagagagaacccaccgccgccaccatggccctgcccgtgacagctctgctgctccctctggctctgctgctgcacgctgccagacccgacatcgtgctgacacaaagcccccctagcctggccatgagcctgggcaagagggccacaatctcctgcagggccagcgagtccgtgaccatcctgggcagccacctgatccactggtaccagcagaagcctggccagccccctaccctgctgatccagctggcctccaacgtgcagacaggagtgcccgctaggttctccggctccggcagcagaaccgactttaccctgaccatcgaccccgtcgaggaggacgacgtggctgtctactattgcctgcagagcaggaccatccccaggaccttcggaggcggcacaaagctggagatcaagggcagcaccagcggctccggaaagcctggcagcggagaaggctccaccaagggacagattcagctggtgcagagcggacccgagctgaagaagcccggcgaaacagtcaagatcagctgcaaagcttccggctacaccttcacagactacagcatcaactgggtgaaaagggcccccggcaagggcctgaaatggatgggctggatcaacaccgagaccagggagcccgcctacgcctacgactttaggggcaggttcgccttcagcctggagacatccgctagcaccgcttacctgcaaattaataacctgaagtacgaggacaccgccacctatttttgtgccctggactacagctacgcaatggactattggggacagggaacctccgtgaccgtgagcagctttgtgcccgtgttcctgcccgccaaacccaccaccacacctgctcctaggccccctacacctgcccctaccatcgcttcccagcccctgagcctcagacctgaagcctgcagacctgctgctggaggcgccgtccacacaaggggactggacttcgcctgtgacatctacatctgggctcctctcgccggcacctgtggagtgctgctgctgtccctggtcatcaccctgtactgcaaccacaggaacagagtgaagttcagcagatccgctgatgcccctgcctatcagcagggccagaaccaactctacaacgagctgaacctgggaaggagggaggagtacgacgtgctcgacaagagaaggggaagggatcctgaaatgggcggcaagcccaggagaaagaaccctcaggagggcctgtacaacgaactgcagaaggataagatggccgaggcctacagcgaaatcggcatgaaaggcgagagaaggagaggcaagggccacgacggactgtaccagggcctgtccaccgccaccaaagacacctacgatgccctgcacatgcaggccctgccccccaggtgatgcccgtcctcaccaagactgactgcctgctgctttgctactgcccgggcccatgagactgacttcccactgctctgcctgcctctccccactgcactggcacagccccgccttgccgctgctgatccattgccggtgtgacccaagcacgcagcaatgcagctcaaaacgcttagcctagccacacccccacgggaaacagcagtgattaacctttagcaataaacgaaagtttaactaagctatactaaccccagggttggtcaatttcgtgccagccacacca

SEQ ID NO: 97 - RNA, 1847 nt, synthetic construct
  source 1..1847: mol_type = other RNA
aggactcttctggtccccacagactcagagagaacccaccgccaccatggcactgccagtcacagcactgctgctgccactggcactgctgctccacgccgccagacccgacatcgtgctgacacaaagcccccctagcctggccatgagcctgggcaagagggccacaatctcctgcagggccagcgagtccgtgaccatcctgggcagccacctgatccactggtaccagcagaagcctggccagccccctaccctgctgatccagctggcctccaacgtgcagacaggagtgcccgctaggttctccggctccggcagcagaaccgactttaccctgaccatcgaccccgtcgaggaggacgacgtggctgtctactattgcctgcagagcaggaccatccccaggaccttcggaggcggcacaaagctggagatcaagggcagcaccagcggctccggaaagcctggcagcggagaaggctccaccaagggacagattcagctggtgcagagcggacccgagctgaagaagcccggcgaaacagtcaagatcagctgcaaggcctccggctacaccttcacagactacagcatcaactgggtgaaaagggcccccggcaagggcctgaaatggatgggctggatcaacaccgagaccagggagcccgcctacgcctacgactttaggggcaggttcgccttcagcctggagacatccgctagcaccgcttacctgcaaattaataacctgaagtacgaggacaccgccacctatttttgtgccctggactacagctacgcaatggactattggggacagggaacctccgtgaccgtgagcagcttcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacaggtccgagagatctcgccttctccattctgactacatgaacatgacgccccgcaggccagggccaacacgagagcattatcagccatatgccccaccacgcgattttgctgcgtaccgctctagagtcgaattctcccggtccgctgacgcccctgcgtatcagcagggtcagaaccaactgtataatgagcttaatcttggaagaagagaggaatacgacgtattggatgagaggcgagggcgcgacccggaaatgggcggagaaccccagaggcgagagaatcctcaagagggactttataacgagctgcaggaagatgagatggcggaagcatacagtgaaataggaatggaaggcgaacggcgcaggggcgaagggcatgatggtctgtatcagggtctttctactgcaacggaagacacgtatgacgccttgcatatgcaggcgctgccccccaggtgatgcccgtcctcaccaagactgactgcctgctgctttgctactgcccgggcccatgagactgacttcccactgctctgcctgcctctccccactgcactggcacagccccgccttgccgctgctgatccattgccggtgtgacccaagcacgcagcaatgcagctcaaaacgcttagcctagccacacccccacgggaaacagcagtgattaacctttagcaataaacgaaagtttaactaagctatactaaccccagggttggtcaatttcgtgccagccacacca

SEQ ID NO: 98 - RNA, 1724 nt, synthetic construct
  source 1..1724: mol_type = other RNA
aggactcttctggtccccacagactcagagagaacccaccgccaccatggcactgccagtcacagcactgctgctgccactggcactgctgctccacgccgccagacccgacatcgtgctgacacagagcccagcaagcctggccgtgagcccaggccagagagccacaatcacatgcagagccagcgagtccgtgtccttcctgggcatcaacctgatccactggtaccagcagaagcccggacagccacccaaactgctgatctactccgccagcaacctgcagtcaggcgtgccagccagattcagcggcagcggcagcggaaccgacttcaccctgacaatcagctcagtggaaccagaagacaccgccaactactactgcctgcagagcagaaccctgcccagaaccttcggccaaggcaccaaggtcgaaatcaagggctccacaagcggcagcggaaagcccggaagcggcgagggaagcaccaagggccagatccagctggtgcagagcggccccgaactgaagaaacccggcggaagcgtgaaaatctcctgcaaggccagcggctacaccttcacctcctacagcatcaactgggtgcgccaggccccaggcaagggcctggaatgggtcggctggatcaacaccgagacaagagagcccgcctacgcccagggcttcacaggcagattcaccttcagcgcagacacaagcaagagcatggcctacctgcagatcaactccctgagagcagaggacaccgccgtctactactgcgccctggactacctctactccctggacttctggggccagggcaccctcgtgaccgtgtccagcttcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacagagtcgaattctcccggtccgctgacgcccctgcgtatcagcagggtcagaaccaactgtataatgagcttaatcttggaagaagagaggaatacgacgtattggatgagaggcgagggcgcgacccggaaatgggcggagaaccccagaggcgagagaatcctcaagagggactttataacgagctgcaggaagatgagatggcggaagcatacagtgaaataggaatggaaggcgaacggcgcaggggcgaagggcatgatggtctgtatcagggtctttctactgcaacggaagacacgtatgacgccttgcatatgcaggcgctgccccccaggtgatgcccgtcctcaccaagactgactgcctgctgctttgctactgcccgggcccatgagactgacttcccactgctctgcctgcctctccccactgcactggcacagccccgccttgccgctgctgatccattgccggtgtgacccaagcacgcagcaatgcagctcaaaacgcttagcctagccacacccccacgggaaacagcagtgattaacctttagcaataaacgaaagtttaactaagctatactaaccccagggttggtcaatttcgtgccagccacacca

SEQ ID NO: 99 - RNA, 1847 nt, synthetic construct
  source 1..1847: mol_type = other RNA
aggactcttctggtccccacagactcagagagaacccaccgccaccatggcactgccagtcacagcactgctgctgccactggcactgctgctccacgccgccagacccgacatcgtgctgacacagagcccagcaagcctggccgtgagcccaggccagagagccacaatcacatgcagagccagcgagtccgtgtccttcctgggcatcaacctgatccactggtaccagcagaagcccggacagccacccaaactgctgatctactccgccagcaacctgcagtcaggcgtgccagccagattcagcggcagcggcagcggaaccgacttcaccctgacaatcagctcagtggaaccagaagacaccgccaactactactgcctgcagagcagaaccctgcccagaaccttcggccaaggcaccaaggtcgaaatcaagggctccacaagcggcagcggaaagcccggaagcggcgagggaagcaccaagggccagatccagctggtgcagagcggccccgaactgaagaaacccggcggaagcgtgaaaatctcctgcaaggccagcggctacaccttcacctcctacagcatcaactgggtgcgccaggccccaggcaagggcctggaatgggtcggctggatcaacaccgagacaagagagcccgcctacgcccagggcttcacaggcagattcaccttcagcgcagacacaagcaagagcatggcctacctgcagatcaactccctgagagcagaggacaccgccgtctactactgcgccctggactacctctactccctggacttctggggccagggcaccctcgtgaccgtgtccagcttcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacaggtccgagagatctcgccttctccattctgactacatgaacatgacgccccgcaggccagggccaacacgagagcattatcagccatatgccccaccacgcgattttgctgcgtaccgctctagagtcgaattctcccggtccgctgacgcccctgcgtatcagcagggtcagaaccaactgtataatgagcttaatcttggaagaagagaggaatacgacgtattggatgagaggcgagggcgcgacccggaaatgggcggagaaccccagaggcgagagaatcctcaagagggactttataacgagctgcaggaagatgagatggcggaagcatacagtgaaataggaatggaaggcgaacggcgcaggggcgaagggcatgatggtctgtatcagggtctttctactgcaacggaagacacgtatgacgccttgcatatgcaggcgctgccccccaggtgatgcccgtcctcaccaagactgactgcctgctgctttgctactgcccgggcccatgagactgacttcccactgctctgcctgcctctccccactgcactggcacagccccgccttgccgctgctgatccattgccggtgtgacccaagcacgcagcaatgcagctcaaaacgcttagcctagccacacccccacgggaaacagcagtgattaacctttagcaataaacgaaagtttaactaagctatactaaccccagggttggtcaatttcgtgccagccacacca

SEQ ID NO: 100 - RNA, 1724 nt, synthetic construct
  source 1..1724: mol_type = other RNA
aggactcttctggtccccacagactcagagagaacccaccgccaccatggccctgcccgtgacagctctgctgctccctctggctctgctgctgcacgctgccagacccgacatcgtgctgacacaaagcccccctagcctggccatgagcctgggcaagagggccacaatctcctgcagggccagcgagtccgtgaccatcctgggcagccacctgatccactggtaccagcagaagcctggccagccccctaccctgctgatccagctggcctccaacgtgcagacaggagtgcccgctaggttctccggctccggcagcagaaccgactttaccctgaccatcgaccccgtcgaggaggacgacgtggctgtctactattgcctgcagagcaggaccatccccaggaccttcggaggcggcacaaagctggagatcaagggcagcaccagcggctccggaaagcctggcagcggagaaggctccaccaagggacagattcagctggtgcagagcggacccgagctgaagaagcccggcgaaacagtcaagatcagctgcaaagcttccggctacaccttcacagactacagcatcaactgggtgaaaagggcccccggcaagggcctgaaatggatgggctggatcaacaccgagaccagggagcccgcctacgcctacgactttaggggcaggttcgccttcagcctggagacatccgctagcaccgcttacctgcaaattaataacctgaagtacgaggacaccgccacctatttttgtgccctggactacagctacgcaatggactattggggacagggaacctccgtgaccgtgagcagcttcgtgcccgtgttcctgccagccaagccaaccacaacaccagcacccagaccaccaacaccagcaccaacaatcgccagccagccactgtccctgaggccagaggcatgcagaccagcagcaggcggagccgtgcacaccagaggactggacttcgcctgcgacatctacatctgggcaccactggccggaacatgcggcgtgctgctgctgagcctggtcatcaccctgtactgcaaccaccggaacagagtcgaattctcccggtccgctgacgcccctgcgtatcagcagggtcagaaccaactgtataatgagcttaatcttggaagaagagaggaatacgacgtattggatgagaggcgagggcgcgacccggaaatgggcggagaaccccagaggcgagagaatcctcaagagggactttataacgagctgcaggaagatgagatggcggaagcatacagtgaaataggaatggaaggcgaacggcgcaggggcgaagggcatgatggtctgtatcagggtctttctactgcaacggaagacacgtatgacgccttgcatatgcaggcgctgccccccaggtgatgcccgtcctcaccaagactgactgcctgctgctttgctactgcccgggcccatgagactgacttcccactgctctgcctgcctctccccactgcactggcacagccccgccttgccgctgctgatccattgccggtgtgacccaagcacgcagcaatgcagctcaaaacgcttagcctagccacacccccacgggaaacagcagtgattaacctttagcaataaacgaaagtttaactaagctatactaaccccagggttggtcaatttcgtgccagccacacca

SEQ ID NO: 101 - protein, 463 aa, synthetic construct
MALPVTALLLPLALLLHAARPDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRVEFSRSADAPAYQQGQNQLYNELNLGRREEYDVLDERRGRDPEMGGEPQRRENPQEGLYNELQEDEMAEAYSEIGMEGERRRGEGHDGLYQGLSTATEDTYDALHMQALPPR

SEQ ID NO: 102 - protein, 504 aa, synthetic construct
MALPVTALLLPLALLLHAARPDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSERSRLLHSDYMNMTPRRPGPTREHYQPYAPPRDFAAYRSRVEFSRSADAPAYQQGQNQLYNELNLGRREEYDVLDERRGRDPEMGGEPQRRENPQEGLYNELQEDEMAEAYSEIGMEGERRRGEGHDGLYQGLSTATEDTYDALHMQALPPR

(19) United States
(12) Patent Application Publication
(10) Pub. No.: US 2026/0265394 A1
(43) Pub. Date: Sep. 10, 2026

(54) INTRACELLULAR SIGNALING AND COSTIMULATORY DOMAINS ADAPTED FOR PROLONGED EXPRESSION OF CHIMERIC ANTIGEN RECEPTORS

(71) Applicant: Cartesian Therapeutics, Inc., Frederick, MD (US)

(72) Inventors: Yi Zhang, Potomac, MD (US); Shaji Daniel, Germantown, MD (US); Atsuo Ochi, Frederick, MD (US); C. Andrew Stewart, Frederick, MD (US); Metin Kurtoglu, Bethesda, MD (US); Murat V. Kalayoglu, Silver Spring, MD (US); Michael S. Singer, Chestnut Hill, MA (US)

(73) Assignee: Cartesian Therapeutics, Inc., Frederick, MD (US)

(21) Appl. No.: 19/166,014

(22) PCT Filed: Mar. 15, 2024
(86) PCT No.: PCT/US2024/020251

§ 371 Date: Sep. 16, 2025

Related U.S. Application Data
(60) Provisional application No. 63/491,038, filed on Mar. 17, 2023.

(51) Int. Cl. C07K 16/28; A61K 9/00; A61K 31/7105; A61K 31/711; A61K 35/12; A61K 35/17; A61K 35/28; A61K 38/19; A61K 40/11; A61K 40/15; A61K 40/31; A61K 40/42; A61P 35/00; C07K 14/705; C12N 5/078; C12N 15/86

(52) U.S. Cl. CPC C07K 16/2878; A61K 9/0019; A61K 31/7105; A61K 31/711; A61K 35/17; A61K 35/28; A61K 38/19; A61K 40/11; A61K 40/15; A61K 40/31; A61K 40/42; A61P 35/00; C07K 14/7051; C07K 14/70521; C07K 16/2803; C12N 5/0634; C12N 15/86; A61K 2035/124; A61K 2239/48; C07K 2317/53; C07K 2317/622; C07K 2319/02; C07K 2319/03

(57) ABSTRACT

Provided herein are proteins that comprise a CD3-zeta intracellular domain, such as Chimeric Antigen Receptors (CARs), such as those specific for BCMA, that have improved properties. Use of the proteins that comprise a CD3-zeta intracellular domain, such as CARs, in immune cells (e.g., T cells), compositions (e.g., CARs and nucleic acid constructs encoding the same), and methods are also contemplated.

### RELATED APPLICATIONS

This application claims priority under 35 U.S.C. § 119(e) to U.S. provisional patent application, U.S. Ser. No. 63/491,038, filed Mar. 17, 2023, the entire contents of which are incorporated herein by reference.

### REFERENCE TO AN ELECTRONIC SEQUENCE LISTING

The contents of the electronic sequence listing (C154070004WO00-SEQ-AZW.xml; Size: 137,935 bytes; and Date of Creation: Mar. 15, 2024) is herein incorporated by reference in its entirety.

### BACKGROUND

A Chimeric Antigen Receptor (CAR) is a synthetic transmembrane protein comprising an extracellular antigen recognition domain (e.g., an antibody single-chain variable fragment), a transmembrane domain, and an intracellular signaling domain (e.g., a T cell signaling domain, e.g., CD3-zeta). When, by artificial means, the CAR is expressed in or by a first cell (e.g., a T cell), the CAR directs the first cell to kill a second cell, such as a cancer cell, wherein the second cell expresses a surface antigen that is, by design, recognized by the CAR's extracellular antigen recognition domain. The cell modified to express the CAR, for example a T cell (such as a CAR T cell), can be administered to a patient to kill tumor cells or other pathogenic cells. Toward this end, CARs have been developed with extracellular antigen recognition domains that specifically bind surface antigens (markers), such as CD19, BCMA, EGFR/HER, CD22, mesothelin, CD123, CD20, PD1, and CD30. CAR-expressing cells, e.g., CAR T cells, have been developed to treat hematologic malignancies, solid tumors, and non-cancerous conditions, such as autoimmune disease. See, for example, Alnefaie et al., “Chimeric Antigen Receptor T-Cells: an overview of concepts, applications, limitations, and proposed solutions” Front. Bioeng. Biotechnol. 2022; 10:797440 (doi 10.3389); and U.S. Pat. No. 10,934,337, each of which is incorporated herein by reference.

### SUMMARY

Nevertheless, efforts to obtain optimal expression of a CAR in a cell of interest, e.g., in a T cell, have been hampered by an unwanted down-modulation or internalization of the CAR after it binds its corresponding antigen. See, for example: Caruso et al. (Cancer Res. 2015; 75:3505); Davenport et al. (Immunol. Res. 2015; 3:483); Walker et al. (Mol. Ther. 2017; 25:2189); Hamieh et al. (Nature 2019; 568:7750); and Li (Immunity 2020; 53:456). This phenomenon can reduce the durability and benefit of a CAR-based therapy. Therefore, there is a need for new CAR molecules that resist, or are less prone to, down-modulation or internalization after they bind their corresponding antigens.

The present disclosure is based on the identification that certain novel modifications to the intracellular domain CD3-zeta (the terms “CD3-zeta,” “CD3-Z,” and “CD3-ζ” are used interchangeably herein) of a CAR (i.e., series of amino acid substitutions) confer upon the CAR: (1) a remarkable degree of maintenance of CAR expression despite (e.g., after) exposure of the CAR to its corresponding antigen (e.g., its intended target); (2) superior target cell cytotoxicity; and/or (3) superior cytokine (e.g., interferon-gamma) secretion. The aforementioned superior experimental performance of the CAR proteins of the present disclosure is in comparison to otherwise identical CAR proteins comprising the wild-type CD3-zeta intracellular domain (for example, SEQ ID NO: 18), where exposure to the CAR's corresponding antigen results in a substantial drop in CAR expression. An observation was also made that some of the amino acid substitutions of the present disclosure conferred superior CAR expression even prior to exposure of the CAR to its corresponding antigen. Again, this was determined by experimental comparison to otherwise identical CAR proteins that comprise the wild-type intracellular CD3-zeta intracellular domain.

Thus in on aspect, provided herein are proteins that is capable of intracellular signaling comprising an intracellular domain, wherein the intracellular domain comprises an intracellular signaling domain, a costimulatory domain, or both, and wherein at least two lysine amino acids of the intracellular domain are substituted by an amino acid independently selected from the group consisting of alanine, aspartate, asparagine, glutamate, glutamine, histidine, leucine, methionine, serine, threonine, and valine.

In some embodiments, the intracellular domain comprises a CD3-zeta domain. In certain embodiments, the intracellular domain comprises a domain that is at least 80%, at least 85%, at least 90%, at least 95%, or at least 99% identical to SEQ ID NO:18 or 61, or 66. In some embodiments, the CD3-zeta intracellular domain is 100% identical to SEQ ID NO: 18, 61, or 66 except for the substituted lysine amino acids.

In some embodiments, at least three lysine amino acids are substituted by an amino acid independently selected from the group consisting of alanine, aspartate, asparagine, glutamate, glutamine, histidine, leucine, methionine, serine, threonine, and valine.

In certain environments, at least four, at least five, at least six, at least seven, or at least eight lysine amino acids are substituted by an amino acid independently selected from the group consisting of alanine, aspartate, asparagine, glutamate, glutamine, histidine, leucine, methionine, serine, threonine, and valine.

In some embodiments, at least nine lysine amino acids are substituted by an amino acid independently selected from the group consisting of alanine, aspartate, asparagine, glutamate, glutamine, histidine, leucine, methionine, serine, threonine, and valine.

In certain embodiments, at least six at least seven, at least eight, or at least nine lysine amino acids are substituted by an amino acid independently selected from the group consisting of alanine, aspartate, and glutamate.

In another aspect of the present disclosure, provided is a CD3-zeta intracellular domain, wherein at least two lysine amino acids of the CD3-zeta intracellular domain are substituted by an amino acid independently selected from the group consisting of alanine, aspartate, asparagine, glutamate, glutamine, histidine, leucine, methionine, serine, threonine, and valine.

In certain embodiments, the intracellular domain comprises a domain that is at least 80%, at least 85%, at least 90%, at least 95%, or at least 99% identical to SEQ ID NO:18, 61, or 66.

In some embodiments, the CD3-zeta intracellular domain is 100% identical to SEQ ID NO: 18, 61, or 66 except for the substituted lysine amino acids.

In certain embodiments, at least three lysine amino acids are substituted by an amino acid independently selected from the group consisting of alanine, aspartate, asparagine, glutamate, glutamine, histidine, leucine, methionine, serine, threonine, and valine.

In some embodiments, at least four, at least five, at least six, at least seven, or at least eight lysine amino acids are substituted by an amino acid independently selected from the group consisting of alanine, aspartate, asparagine, glutamate, glutamine, histidine, leucine, methionine, serine, threonine, and valine.

In certain embodiments, at least nine lysine amino acids are substituted by an amino acid independently selected from the group consisting of alanine, aspartate, asparagine, glutamate, glutamine, histidine, leucine, methionine, serine, threonine, and valine.

In some embodiments, at least six at least seven, at least eight, or at least nine lysine amino acids are substituted by an amino acid independently selected from the group consisting of alanine, aspartate, and glutamate.

In certain embodiments, the CD3-zeta intracellular domain is a domain of a protein.

In some embodiments, wherein the CD3-zeta intracellular domain is a domain of a transmembrane protein.

In some embodiments, the CD3-zeta intracellular domain is a domain of an intercellular signaling protein.

In certain embodiments, the CD3-zeta intracellular domain is a domain of a CAR.

In one aspect, provided herein is a protein comprising the CD3-zeta intracellular of the present disclosure.

In another aspect of the present disclosure, provided is a protein comprising a CD3-zeta intracellular domain, wherein the CD3-zeta intracellular domain is at least 80%, at least 85%, at least 90%, at least 95%, or at least 99% identical to SEQ ID NO:18, wherein at least two lysine amino acids of SEQ ID NO: 18 are either (a) deleted or (b) substituted by an amino acid independently selected from the group consisting of alanine, aspartate, asparagine, glutamate, glutamine, histidine, leucine, methionine, serine, threonine, and valine.

In some embodiments, the CD3-zeta intracellular domain is 100% identical to SEQ ID NO: 18 except for the deleted or substituted lysine amino acids.

In certain embodiments, at least three, at least four, at least five, at least six, at least seven, at least eight, or at least nine lysine amino acids of SEQ ID NO: 18 are either (a) deleted or (b) substituted by an amino acid independently selected from the group consisting of alanine, aspartate, and glutamate.

In some embodiments, at least three, at least four, at least five, at least six, at least seven, at least eight, or at least nine lysine amino acids of SEQ ID NO: 18 are substituted by alanine.

In certain embodiments, at least three, at least four, at least five, at least six, at least seven, at least eight, or at least nine lysine amino acids of SEQ ID NO: 18 are substituted by aspartate.

In some embodiments, at least three, at least four, at least five, at least six, at least seven, at least eight, or at least nine lysine amino acids of SEQ ID NO: 18 are substituted by glutamate.

In another aspect, provided is a protein comprising an amino acid sequence that is at least 80% identical to any one of SEQ ID NOs: 2-7, 19-66, and 68-86.

In some embodiments, the protein comprising an amino acid sequence that is, at least 85%, at least 90%, at least 95%, or at least 99% identical to any one of SEQ ID NOs: 2-7, 19-66, and 68-86.

In certain embodiments, the protein is a Chimeric Antigen Receptor (CAR).

In some embodiments, the protein further comprising a costimulatory domain.

In certain embodiments, the costimulatory domain is selected from the group consisting of CD8-alpha domains, 41BB domains, CD28 domains, FcR gamma domains, CD27 domains, OX40 domains, CD30 domains, CD40 domains, PD-1 domains, ICOS domains, LFA-1 domains, CD2 domains, CD7 domains, LIGHT domains, NKG2C domains, and B7 H3 domains, and any variants thereof.

In some embodiments, the costimulatory domain is CD28.

In some embodiments, the costimulatory domain is 41BB.

In certain embodiments, at least one lysine amino acids of the costimulatory domain are substituted by an amino acid independently selected from the group consisting of alanine, aspartate, asparagine, glutamate, glutamine, histidine, leucine, methionine, serine, threonine, and valine.

In some embodiments, at least two lysine amino acids of the costimulatory domain are substituted by an amino acid independently selected from the group consisting of alanine, aspartate, and glutamate.

In certain embodiments, the protein further comprising an extracellular antigen binding domain.

In some embodiments, the extracellular antigen binding domain binds CD19, BCMA, EGFR/HER, CD22, mesothelin, CD123, CD20, PD1, or CD30.

In certain embodiments, the extracellular antigen binding domain binds BCMA.

In some embodiments, the extracellular antigen binding domain binds CD19.

In certain embodiments, the extracellular antigen binding domain is a scFv.

In some embodiments, the protein further comprising a transmembrane domain.

In certain embodiments, the transmembrane domain comprises the transmembrane region of MHC class I molecules, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, or a receptor that specifically binds with CD83.

In certain embodiments, the protein further comprising a hinge region.

In some embodiments, the protein of the present disclosure further comprising a leader domain.

In certain embodiments, the protein further comprises one or more spacer sequences between one or more of the domains.

In certain embodiments, the spacer sequences is a polypeptide linker.

In another aspect, the present disclosure provides a nucleic acid construct encoding the protein disclosed herein.

In some embodiments, the nucleic acid construct is RNA.

In certain embodiments, the nucleic acid construct is DNA.

In one aspect, provided herein a vector encoding the protein described herein.

In certain embodiments, the vector comprises the nucleic acid construct disclosed herein.

In some embodiments, the vector is a viral vector.

In another aspect, provided herein a composition comprising the protein disclosed herein, the nucleic acid construct described herein, or the vector described herein,

In another aspect of the present disclosure, provided herein a pharmaceutical composition comprising the composition of the protein described herein, the nucleic acid construct described herein, or the vector described herein.

In certain embodiments, the pharmaceutical composition further comprising s pharmaceutically acceptable excipient.

In one aspect, provided herein a cell comprising the protein of the present disclosure.

In another aspect, the present disclosure provides a cell comprising the nucleic acid construct or the vector of describe herein.

In some embodiments, the cell is a human cell.

In certain embodiments, the cell is an immune cell.

In some embodiments, the cell is a T-cell, CD3+ cell, CD8+ cell, CD4+ cell, NK cell, stem cell, hematopoietic stem cell, or mesenchymal stem cell.

In one aspect, provided is a method for producing a cell therapy for treating a disease, the method comprising transfecting a plurality of cells with vector described herein.

In another aspect of the present disclosure, provided is a method of treating a disease in a subject in need thereof, the method comprising administering to the subject the cell described herein.

In some embodiments, the cells are human cells.

In certain embodiments, the cells are immune cells.

In some embodiments, the cells are T cells.

In certain embodiments, the cells are CD3+ cells.

In some embodiments, the cells are CD8+ cells.

In certain embodiments, the cells are CD4+ cells.

In certain embodiments, the cells are NK cells.

In certain embodiments, the cells are stem cells.

In some embodiments, the stem cells are hematopoietic stem cells.

In some embodiments, the stem cells are mesenchymal stem cells.

In certain embodiments, the method further comprising a cytokine.

In some embodiments, the disease is cancer, an autoimmune condition, or an allergic condition.

In certain embodiments, the disease is myeloma.

In some embodiments, the disease is myeloma

In certain embodiments, the disease myasthenia gravis (MG) disease

In some embodiments, the method is characterized by increased cellular secretion of a cytokine.

In certain embodiments, the secreted cytokine is interferon gamma.

In some embodiments, the method is characterized by the selective killing of cancer cells.

In some embodiments, the method is characterized by the selective killing of immune cells.

In certain embodiments, the method is characterized by the selective killing of BCMA+ or CD19+ cells.

In one aspect, provided is the use of proteins disclosed herein, the nucleic acid construct described herein, the viral vector disclosed herein, or the cell described herein for the treatment of cancer.

In another aspect, provided is a kit comprising one or more of the proteins disclosed herein, the nucleic acid construct provided herein, the viral vector described herein, or the cell disclosed herein.

### BRIEF DESCRIPTION OF THE DRAWINGS

FIG. 1A shows CAR expression in the absence of the target ligand (left) and presence of the target ligand (right) measured as median fluorescent intensity (MFI) for CAR T cells generated with wild-type CD3-zeta intracellular domain sequence (SEQ ID NO: 13) and with lysine-mutant constructs (SEQ ID NOs: 14-17).

FIG. 1B shows expression of interferon-gamma in supernatants of co-cultures between CAR T cells generated with lysine-mutant constructs (SEQ ID NOs: 14-17) or wild-type CAR T cells (SEQ ID NO: 13) and BCMA+ MM1S multiple myeloma analyzed by specific ELISA. Prior to co-culture, CAR T cells were pre-exposed (grey) or not pre-exposed (black) to BCMA+ cells.

FIG. 1C shows cytotoxicity of pre-exposed CAR T cells generated with constructs encoding lysine-mutant CAR (SEQ ID NOs: 14-17) and wild-type CAR (SEQ ID NO: 13) against MM1S-GFP target cells expressing BCMA ligand. Cytotoxicity was evaluated by co-culture at various effector:target ratios.

FIG. 2A shows CAR expression in the absence of the target ligand (left) and presence of the target ligand (right) measured as median fluorescent intensity (MFI) for CAR T cells generated with wild-type constructs (SEQ ID NO: 18 and 61) and mutated constructs (SEQ ID NOs: 19-27 and 43).

FIG. 2B shows expression of interferon-gamma during co-culture with MM1S target cells for CAR T cells generated with various test constructs (SEQ ID NOs: 19-27 and 43) and wild-type CAR T (SEQ ID NOs: 18 and 61) cells.

FIG. 2C shows CAR expression in the absence of the target ligand (left) and presence of the target ligand (right) measured as median fluorescent intensity (MFI) for CAR T cells generated with wild-type constructs (SEQ ID NO: 18 and 61) and mutated constructs (SEQ ID NOs: 34-43).

FIG. 2D shows expression of interferon-gamma during co-culture with MM1S target cells for CAR T cells generated with various test constructs (SEQ ID NOs: 34-43) and wild-type CAR T (SEQ ID NOs: 18 and 61) cells.

FIG. 2E shows CAR expression in the absence of the target ligand (left) and presence of the target ligand (right) measured as median fluorescent intensity (MFI) for CAR T cells generated with wild-type constructs (SEQ ID NO: 18 and 61) and mutated constructs (SEQ ID NOs: 28-33 and 43).

FIG. 2F shows expression of interferon-gamma during co-culture with MM1S target cells for CAR T cells generated with various test constructs (SEQ ID NOs: 28-33 and 43) and wild-type CAR T (SEQ ID NOs: 18 and 61) cells.

FIG. 3A shows CAR expression measured as median fluorescent intensity (MFI) in the absence of BCMA ligand (left) and the presence of BCMA ligand (right) by CAR T cells expressing various test constructs (SEQ ID NOs: 43-56) and CAR T cells expressing wild-type constructs (SEQ ID NOs: 18 and 61).

FIG. 3B shows expression of interferon-gamma during co-culture with MM1S target cells for CAR T cells generated with various test constructs (SEQ ID NOs: 43-56) and wild-type CAR T (SEQ ID NOs: 18 and 61) cells.

FIG. 4A shows CAR expression measured as median fluorescent intensity (MFI) in the absence of BCMA ligand (left) and the presence of ligand (right) by CAR T cells generated with various test constructs (SEQ ID NOs: 43 and 57-60) and CAR T cells generated with wild-type construct (SEQ ID NO: 18).

FIG. 4B shows expression of interferon-gamma upon exposure to MM1S target cells for CAR T cells generated with various test constructs (SEQ ID NOs: 43 and 57-60) and wild-type construct (SEQ ID NO: 18).

FIG. 4C shows cytotoxicity of CAR T cells generated with separate constructs comprising wild-type CD3-zeta (SEQ ID NO:61), or mutated CD3-zeta only, CD28-CD3-zeta, and 41BB-CD3-zeta signaling domains (SEQ ID NOs: 43, 57, and 59, respectively). Cytotoxicity was evaluated by pre-exposure of cells to MM1S followed by a cytotoxicity assay against BCMA+ MM1S-GFP cells for 72 hours at various effector:target ratios.

FIG. 5A shows CAR expression measured as median fluorescent intensity (MFI) in the absence of BCMA ligand (left) and the presence of BCMA ligand (right) by CAR T cells expressing variations of the anti-BCMA CAR protein (SEQ ID NOs: 43, 56-58, and 62-65) and CAR T cells expressing wild-type construct (SEQ ID NO: 18).

FIG. 5B shows cytotoxicity of CAR T cells generated with various constructs (SEQ ID NOs: 18, 43, 57, 58, and 62). Cytotoxicity was evaluated by pre-exposure of cells to MM1S followed by a cytotoxicity assay against BCMA+ MM1S-GFP cells for 72 hours at various effector:target ratios.

FIG. 5C shows cytotoxicity of CAR T cells generated with various constructs (SEQ ID NOs: 56 and 63-65). Cytotoxicity was evaluated by pre-exposure of cells to MM1S followed by a cytotoxicity assay against BCMA+ MM1S-GFP cells for 72 hours at various effector:target ratios.

FIGS. 6A-6B show flow cytometric evaluation of activation of BCMA CAR T (SEQ ID NO: 43) or control CD8+ T cells without CAR using activation induced markers CD69 and CD137 (41BB). Activation of BCMA CAR T (SEQ ID NO: 43) and control CD8+ T cells was evaluated in response to co-culturing with plasmablasts (FIG. 6A) and plasmacytoid dendritic cells (FIG. 6B).

FIGS. 7A-7B show bioluminescence of mice carrying MM1S-fluc myeloma and treated with control CD8+ T cells, or CAR T cells expressing CAR of SEQ ID NOs: 5 and 68. FIG. 7A shows bioluminescence measurement of individual mice on Day 9 and Day 12. FIG. 7B shows summary statistics of mice in each group at given timepoints. The arrow indicates the day of CAR T cell administration. Mean±S.D for n=4.

FIGS. 8A-8B show bioluminescence (total flux) of mice carrying MM1S-fluc myeloma and treated with control CD8+ T cells, or CAR T cells containing anti-BCMA CAR with K-E-mutated intracellular CD28-CD3-zeta (SEQ ID NO: 65) or wild-type intracellular CD28-CD3-zeta (SEQ ID NO: 66) signaling domains. FIG. 8A shows summary statistics of bioluminescence (photons/second) for mice treated with 12.5 million CAR T or control cells. The arrow indicates the day of CAR T cell administration. FIG. 8B shows bioluminescence data from individual mice on Day 21 in groups treated with 2 million or 12.5 million CAR T cells generated with anti-BCMA CAR with K-E-mutated intracellular CD28-CD3-zeta (SEQ ID NO: 65) or wild-type intracellular CD28-CD3-zeta (SEQ ID NO: 66) signaling domains. Mean±S.D for n=4.

FIG. 8C shows flow cytometric evaluation of whole blood and bone marrow of mice administered with CAR T cells generated with IVT mRNA construct SEQ ID NO: 68 encoding a signaling domain of SEQ ID NO: 65. Cells were stained with antibodies against human CD45, CD3, and recombinant BCMA-APC and evaluated by flow cytometry for expression of CAR. Data show expression of CD3 and CAR on CD45-gated cells.

FIG. 9A shows the cytotoxicity of anti-BCMA CAR T cells from two myasthenia gravis (MG) disease donors prepared using the SEQ ID NO: 68 CAR construct and SEQ ID NO: 94 (anti-PSMA CAR as negative control) against autologous plasma cells differentiated from MG patients.

FIG. 9B shows donor variation in interferon-gamma cytokine production post cytotoxicity.

FIG. 9C shows functionality of MG donor CAR T cells evaluated using malignant MM1S-GFP cell line.

FIG. 9D shows Interferon-gamma cytokine production of MG donor CAR T cells evaluated using malignant MM1S-GFP cell line.

FIG. 10A shows apparent affinity of anti-BCMA CAR to soluble BCMA.

FIG. 10B shows expression of anti-CD19 CAR by CAR T cells expressing CAR containing CD28-CD3-zeta intracellular domains with wild-type sequences or mutant sequences containing lysines mutated to glutamic acid following culture in the absence or presence of BCMA+ MM1S cells.

### DEFINITIONS

As used herein and in the claims, the singular forms “a,” “an,” and “the” include the singular and the plural reference unless the context clearly indicates otherwise. Thus, for example, a reference to “an agent” includes a single agent and a plurality of such agents.

The terms “allergy” and “allergic”, as used herein, refer to a medical condition involving an abnormal hypersensitivity reaction to an ordinarily harmless substance, i.e., an allergen. Exemplary allergic conditions include anaphylaxis, asthma, food allergy, stinging insect allergy, drug allergy, allergic rhinitis, urticaria, angioedema, eczema, atopic dermatitis, contact dermatitis, and eosinophilic esophagitis.

The amino acids in a polypeptide sequence can be identified by their unabbreviated names or by three-letter or single-letter abbreviations, which are known in the art, and such identifiers are used interchangeably herein. The naturally occurring amino acids include Alanine (Ala) (A); Arginine (Arg) (R); Asparagine (Asn) (N); Aspartate (Asp) (D); Cysteine (Cys) (C); Glutamine (Gln) (Q); Glutamate (Glu) (E); Glycine (Gly) (G); Histidine (His) (H); Isoleucine (Ile) (I); Leucine (Leu) (L); Lysine (Lys) (K); Methionine (Met) (M); Phenylalanine (Phe) (F); Proline (Pro) (P); Serine (Ser) (S); Threonine (Thr) (T); Tryptophan (Trp) (W); Tyrosine (Tyr) (Y); and Valine (Val) (V). “Aspartate” includes aspartate and aspartic acid. “Glutamate” includes glutamate and glutamic acid. The basic amino acids include lysine, arginine, and histidine and are positively charged at neutral pH.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (e.g., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, nanobodies, linear antibodies, single chain antibodies, and any other modified configuration of the immunoglobulin molecule that comprises an antigen recognition site of the required specificity, including glycosylation variants of antibodies, amino acid sequence variants of antibodies, and covalently modified antibodies. The term “antibody” refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. Non-limiting embodiments thereof are discussed below.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG 3, IgG4, IgA1 and IgA2) or subclass.

The term “antigen-binding portion” of an antibody (or simply “antibody portion”), as used herein, refers to one or more fragments of an antibody that retain the ability to specifically bind to an antigen (e.g., BCMA). It has been shown that the antigen-binding function of an antibody can be performed by fragments of a full-length antibody. Such antibody embodiments may also be bispecific, dual specific, or multi-specific formats; specifically binding to two or more different antigens. Multispecific, dual specific, and bispecific antibody constructs are well known in the art and described and characterized in Kontermann (ed.), Bispecific Antibodies, Springer, NY (2011), and Spiess et al., Mol. Immunol. 67(2):96-106 (2015).

Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., PCT publication WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5).

The term “synthetic antibody,” as used herein, refers an antibody which is generated using recombinant DNA technology, such as, for example, an antibody expressed by a viral vector. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

In some embodiments, the term “antigen” or “Ag” as used herein is defined as a molecule that provokes an immune response. This immune response may involve either antibody production, or the activation of specific immunologically competent cells, or both.

The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “tumor antigen” as used herein refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 on B cells. In some embodiments, a tumor antigen is a cell surface molecule that is overexpressed in a cancer cell in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. Examples of tumor antigens include but are not limited to, BCMA, CD19, EGFR/HER, CD22, mesothelin, CD123, CD20, PD1, and CD30.

The term “anti-tumor effect,” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the present disclosure in prevention of the occurrence of tumor in the first place.

The term “autoimmune” refers to a disease or illness wherein an individual's immune system, or a component thereof, attacks that individual's normal cells or body tissue(s). An autoimmune disease can be mediated by an autoantibody, i.e., an antibody produced by an individual that recognizes an antigen of that individual's own cells or tissue(s). Exemplary autoimmune diseases include myasthenia gravis, systemic lupus erythematosus (SLE), rheumatoid arthritis, blistering skin diseases, e.g., pemphigus, psoriasis, inflammatory bowel disease, celiac sprue, pernicious anemia, idiopathic thrombocytopenia purpura, scleroderma, Graves disease, Sjögren syndrome, Goodpasture syndrome, multiple sclerosis, and type 1 diabetes.

The term “autologous,” as used herein, is meant to refer to any material derived from the same individual to which it is later to be re-introduced into the individual.

The term “allogeneic,” as used herein, refers to a graft derived from a different animal of the same species. “Xenogeneic” refers to a graft derived from an animal of a different species.

The term “cancer,” as used herein, is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In some embodiments, the cancer is a cancer that expresses BCMA. Exemplary cancers that express BCMA include multiple myeloma, Hodgkin lymphoma, non-Hodgkin lymphoma, chronic lymphocytic leukemia (CLL), and glioblastoma. In some embodiments, cancer refers to multiple myeloma. Multiple myeloma is a cancer of plasma cells. Multiple myeloma can be diagnosed with blood tests (serum protein electrophoresis, serum free kappa/lambda light chain assay), bone marrow examination, urine protein electrophoresis, and/or X-rays of commonly involved bones. In some embodiments, cancer refers to Hodgkin's lymphoma (HL). HL is a cancer of B cells.

An “effective amount” refers to the amount of a therapy which is sufficient to reduce or ameliorate the severity and/or duration of a disorder or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent).

As used herein, the term “exogenous” refers to any material introduced from or produced outside an organism, cell, tissue or system.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein, “CD3-zeta intracellular domain” refers to a protein domain that is the intracellular portion of a CD3-zeta protein. In the human, for example, the CD3-zeta intracellular domain corresponds, corresponds approximately, or is similar, to amino acids 351 to 463 as sequentially numbered in the full-length CD3-zeta amino acid sequence of SEQ ID NO: 1 and corresponds approximately, or is similar to the partial CD3-zeta amino acid sequence of SEO ID NO: 18. The definition of “corresponds approximately” is as follows: because the boundary between one protein domain and another (e.g., between a CD3-zeta transmembrane domain and intracellular domain) is not necessarily strictly defined, “CD3-zeta intracellular domain” can consist of more or fewer amino acids (e.g., between 1 and 10 amino acids more or fewer on each of the amino- and carboxy sides of the sequence as listed, numerically delimited, or otherwise specified) than those described hereinabove, yet still correspond approximately to those described hereinabove, i.e., the amino acid sequence of SEQ ID NO: 18 or amino acids 351-463 of SEQ ID NO: 1. Furthermore, the term “CD3-zeta intracellular domain” is intended to include all allelic variants and naturally-occurring or artificial mutations of CD3-zeta that are not contrary to the amino acid substitutions of the present disclosure described herein.

The term “immunoglobulin” or “Ig,” as used herein is as a class of proteins, which function as antibodies, and the term has it usual meaning in the art.

Where used herein with reference to the selection of two or more amino acids from a list, e.g., a Markush group, “independently selected” means that selection of one amino acid from the list is, or can be, independent of the selection of each successive amino acid from the list, and so forth, and that the first and successive selections can be different or the same.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Unless otherwise specified, a “nucleotide sequence or nucleic acid encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some versions contain an intron(s).

By the term “modulate” or “modulating,” as used herein, is meant mediating a detectable increase or decrease in the level of a response compared with the level of a response in the absence of a treatment or compound, and/or compared with the level of a response in an otherwise identical situation. The term encompasses perturbing and/or affecting a native signal or response thereby mediating a beneficial effect.

The term “linker,” as used herein, refers to a bond (e.g., covalent bond), chemical group, or a molecule linking two molecules or moieties, e.g., two domains of a fusion protein, such as, for example, a nuclease-inactive Cas9 domain and a nucleic acid-editing domain (e.g., an adenosine deaminase). Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

“Parenteral” administration of an immunogenic composition includes, e.g., subcutaneous (s.c), intravenous (i.v.), intramuscular (i.m.), or intrasternal injection, or infusion techniques.

The terms “patient,” “subject,” and “individual” are used interchangeably herein, and refer to any animal, or cells thereof whether in vitro or in situ, amenable to the methods described herein. In some embodiments, the patient, subject or individual is a human. Other examples include dogs, cats, mice, rats, and transgenic species thereof. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development. In some embodiments, the subject has cancer (e.g., multiple myeloma). In other embodiments, the subject is a healthy volunteer.

The term “specifically binds” or “specific for”, as used herein with respect to an antigen recognition domain, e.g., an antibody, e.g., an scFv, means a protein or domain thereof that recognizes a specific antigen (or surface marker) but does not substantially recognize or bind other molecules in a sample (or, in certain contexts, in the body of an individual). For example, an antibody that specifically binds to an antigen from one species may also bind to that antigen from one or more species, but such cross-species reactivity does not itself alter the classification of an antibody as specific. An antibody that specifically binds to an antigen may also bind to different allelic forms of the antigen. However, such cross reactivity does not itself alter the classification of an antibody as specific. In some instances, the terms “specific binding” or “specifically binding,” refers to the interaction of an antibody, a protein (or a domain thereof), or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

Where used herein with reference to an amino acid or an amino acid's position in an amino acid sequence, “substituted” means that one type of amino acid is substituted for another at the same (or corresponding) position in the amino acid sequence. Corresponding amino acid positions in two or more similar sequences, e.g., a wild-type sequence and a substituted sequence, can be ascertained by aligning those sequences by readily available informatics tools, e.g., BLAST. Where two similar proteins have sequences that have or comprise one or more amino acid deletions or insertions with reference to each other, an optimal alignment can be obtained, e.g., with BLAST, by inclusion of one or more gaps, and corresponding amino acid positions can be identified between the two proteins. Where used herein with reference to an amino acid substitution in a sequence, “mutation”, “mutate” and “mutated” refer to an amino acid substitution, typically one that changes the wild-type sequence. Where used herein with reference to modification of an amino acid sequence, “mutation”, “mutate” and “mutated” refer to a substitution of one or more amino acids in the sequence. A mutation can be artificially created. Where used herein with reference to modification of an amino acid sequence, “deletion”, “delete” and “deleted” mean the removal of one or more amino acids from the sequence, typically from the wild-type sequence. In this disclosure, amino acid substitutions or mutations can be described by text such as “X-Z substitution”, “X-Z mutation” and the like, where “X” refers to one or more positions in a first sequence occupied by the amino acid “X”, and “Z” refers to a second sequence wherein such positions are substituted with, or mutated to, the amino acid “Z”. Generally, but not necessarily if the context otherwise indicates, the first sequence is a reference (e.g., wild-type) sequence, and the second sequence is a modified sequence (e.g., modified sequences of the inventive). In some embodiments, the formula “X-Z substitution” refers to only one amino acid position; in some embodiments, it refers to more than amino acid position; and in some embodiments, it refers to all amino acid positions in a sequence (or specified portion thereof) occupied by the amino acids “X” or “Z” as the case may be; in all such cases, the number of amino acid positions where such substitution(s) or mutation(s) occur clear from the context. Thus, for example, a “K-A” or “Lys-Ala” substitution at 9 amino acid positions means that each of 9 positions that occupies a lysine in a first sequence is substituted by an alanine in a second sequence.

As used herein, unless otherwise clear from the context, the term “surface marker” means an antigen or other molecular moiety present on the surface of a cell to which a CAR can specifically bind. Examples of useful surface markers BCMA, CD19, EGFR/HER, CD22, mesothelin, CD123, CD20, PD1, and CD30. A tumor antigen, which is an antigen specific or relatively specific to a cancerous cell, can serve as surface marker. Many (but not all) surface markers are membrane-bound proteins or domains thereof, which can include glycosylation and other post-translational modifications.

As used herein, unless otherwise clear from the context, the terms “target” and derivatives such as “target cell surface marker” refer to a surface marker or a cell, tissue, or tumor that is specifically bound by a CAR. In such cases where the target refers to a cell, tissue, or type of tumor, such cell, tissue, or tumor typically expresses (i.e., displays) a surface marker that is specifically bound by a CAR. Thus, as used herein, a “target cell” refers to a cell that is specifically bound by a particular CAR or CAR-expressing cell, e.g., a CAR T cell.

The term “therapeutic” as used herein means a treatment and/or prophylaxis. A therapeutic effect is obtained by suppression, remission, or eradication of a disease state.

The term “therapeutically effective amount” as used herein, refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated. A therapeutically effective amount does not need to be an amount required for clinical efficacy.

As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

### DETAILED DESCRIPTION OF CERTAIN EMBODIMENTS

### Overall CAR Structure

In one aspect, the present disclosure provides novel CARs that each comprise a modified CD3-zeta intracellular domain, i.e., where certain amino acid residues are selectively modified or mutated with respect to the wild-type sequence, to obtain CARs that provide better and more durable expression and CAR-mediated cellular function, such as cytotoxicity and cytokine secretion.

The overall design of CAR proteins is known in the art. See, for example, Alnefaie, supra, and U.S. Pat. No. 10,934,337, the entire contents of which are hereby incorporated by reference. In some embodiments, a CAR of the present disclosure comprises an extracellular antigen-binding domain specific for a particular surface antigen (marker) (e.g., CD19, BCMA, EGFR/HER, CD22, mesothelin, CD123, CD20, PD1, or CD30), a transmembrane domain, and an intracellular (T-cell signaling) domain of the disclosure, as described herein. Each intracellular domain of the present disclosure comprises a modified CD3-zeta intracellular domain, as disclosed hereinbelow. Optionally, the intracellular domain can further comprise a CD8-alpha protein, a CD28 protein, an FcR gamma protein, a CD27 protein, an OX40 protein, a 4-1BB protein, a CD30 protein, a CD40 protein, PD-1 protein, an ICOSprotein, an LFA-1 protein, a CD2 protein, a CD7 protein, a LIGHT protein, an NKG2C protein, a B7 H3 protein—or a modified version or a portion of any of them—or other costimulatory domains known for use in CARs, and combinations thereof.

Generally, for purposes of CAR construction, wherein a class of suitable extracellular (antigen-binding) domains is known and a separate class of suitable intracellular domains is known, any particular species of such extracellular domain can be combined (via a transmembrane domain) with any particular species of such intracellular domain, to obtain an operable CAR. Thus, where a functional CAR is provided, a different species of extracellular domain can be substituted in that CAR, for example, to confer upon the CAR binding specificity for a particular antigen, i.e., a target cell surface marker. Likewise, where a functional CAR is provided, a different species of intracellular domain can be substituted in that CAR, for example, to affect other CAR properties, as described herein. This phenomenon is beneficial to the present disclosure, as the novel CD3-zeta intracellular domains of the present disclosure as described herein are typically suitable, without limitation, for use in any CAR comprising a CD3-zeta intracellular domain, including many CARs hitherto described. Moreover, the novel CD3-zeta intracellular domains of the present disclosure are suitable in any system that comprises a CD3-zeta intracellular domain. Thus, for example, wherever in this disclosure novel CD3-zeta intracellular domains are described with respect to a BCMA-specific CAR, those same novel CD3-zeta intracellular domains are suitable for use in CARs comprising a different extracellular antigen recognition domain that binds a different surface antigen, such as, but not necessarily limited to CD19, EGFR/HER, CD22, mesothelin, CD123, CD20, PD1, or CD30.

In some embodiments, between the extracellular domain and the transmembrane domain of the CAR, or between the transmembrane domain and the intracellular domain of the CAR, there may be incorporated a spacer and/or hinge domain. As used herein, the term “spacer domain” generally refers to any oligo- or polypeptide that functions to link the transmembrane domain to, either the extracellular domain or, the intracellular domain in the polypeptide chain. Spacer domains for use in CARs are known in the art. See, e.g., U.S. Pat. No. 10,934,337.

In some embodiments, a CAR of the present disclosure comprises the structure NH₂-[extracellular antigen-binding domain]-[transmembrane domain]-[intracellular domain of the present disclosure]-COOH. In some embodiments, the CAR comprises the structure NH₂-[extracellular antigen-binding domain]-[hinge region]-[transmembrane domain]-[intracellular domain of the present disclosure]-COOH. In some embodiments, the CAR comprises one or more spacer sequences. In some embodiments, each instance of “]-[” indicates the optional presence of a spacer sequence.

In some embodiments, the intracellular domain comprises an CD3-zeta protein of the present disclosure and optionally comprises a CD8-alpha protein, a CD28 protein, an FcR gamma protein, a CD27 protein, an OX40 protein, 41BB protein, a CD30 protein, a CD40 protein, PD-1 protein, an ICPS protein, an LFA-1 protein, a CD2 protein, a CD7 protein, a LIGHT protein, an NKG2C protein, a B7 H3 protein—or a portion of any of them—other intracellular costimulatory molecules known for use in CARs, and any combination thereof. In some embodiments, an inventive CAR of the present disclosure comprises at least one of the following structures:
NH₂-[extracellular antigen-binding domain]-[transmembrane domain]-[intracellular domain]-COOH;
NH₂-[extracellular antigen-binding domain]-[hinge region]-[transmembrane domain]-[intracellular domain]-COOH;
NH₂-[signal peptide]-[extracellular antigen-binding domain]-[transmembrane domain]-[intracellular domain]-COOH; or
NH₂-[signal peptide]-[extracellular antigen-binding domain]-[hinge region]-[transmembrane domain]-[intracellular domain]-COOH.

In some embodiments, the CAR comprises a intracellular domain having an arrangement selected from one of the following exemplary, non-limiting arrangements: NH₂-[CD3-zeta intracellular domain of the present disclosure]-COOH; NH₂-[CD28]-[CD3-zeta intracellular domain of the present disclosure]-COOH; NH₂-[41BB]-[CD3-zeta intracellular domain of the present disclosure]-COOH; NH₂-[CD27]-[CD3-zeta intracellular domain of the present disclosure]-COOH; NH₂-[CD40]-[CD3-zeta intracellular domain of the present disclosure]-COOH; NH₂-[ICOS]-[CD3-zeta intracellular domain of the present disclosure]-COOH; NH₂-[CD40L]-[CD3-zeta intracellular domain of the present disclosure]-COOH; NH₂-[OX40]-[CD3-zeta intracellular domain of the present disclosure]-COOH; or NH₂-[41BB]-[OX40]-[CD3-zeta intracellular domain of the present disclosure]-COOH.

In some embodiments, the above exemplary, non-limiting arrangements are from left to right, N-terminus to C-terminus of the CAR. In some embodiments, each instance of “]-[” indicates the optional presence of a spacer sequence.

In some embodiments, a CAR is designed with a leader domain (also referred to as a “signal peptide”) for directing the translated chimeric protein to the membrane. In some embodiments the CAR comprises a leader sequence at the amino terminus of the CAR protein. For example, the CAR can comprise a leader sequence at the N terminus of the extracellular antigen-binding domain, wherein the leader sequence is optionally selected for its tendency or capacity to be cleaved from the antigen-binding domain during cellular processing and localization of the CAR to the cellular membrane. Leader domains are described further in U.S. Pat. No. 10,934,337.

Transmembrane domains for use in CARs are also described in U.S. Pat. No. 10,934,337. The transmembrane domain may be derived either from a naturally occurring sequence or may be synthetic. Where the source is natural, the domain may be derived from any membrane-bound or transmembrane protein, provided that the transmembrane domain permits signaling to the intracellular domain(s) whenever the CAR has bound to a target. A transmembrane domain of particular use in this invention may include at least the transmembrane region(s) of e.g., the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, CD154. In some embodiments, a transmembrane domain may include at least the transmembrane region(s) of a costimulatory molecule, e.g., MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83. In some embodiments, the transmembrane domain may be synthetic, in which case it will comprise predominantly hydrophobic residues such as leucine and valine. In some embodiments, one or both ends of a synthetic transmembrane domain comprise a triplet of phenylalanine, tryptophan and valine. Optionally, a short oligo- or polypeptide linker, e.g., between 2 and 10 amino acids in length, may form the linkage between the transmembrane domain and the intracellular signaling domain of the CAR. A glycine-serine doublet provides an exemplary suitable linker.

In some embodiments, the transmembrane domain in the CAR of the present disclosure is a CD8 transmembrane domain or a CD28 transmembrane domain. Sequences of CD8 for this purpose are known in the art and set forth in PCT Pub No. WO 2014/055771, which is incorporated herein by reference.

### Intracellular Domain

A CAR protein, or any other protein that comprises a CD3-zeta intracellular domain, of the present invention comprises a novel intracellular domain that is a substituted, modified, or mutated version of the CD3-zeta intracellular domain.

In some embodiments, the novel intracellular domain comprises a sequence that is similar to that of the wild-type CD3-zeta intracellular domain sequence (SEQ ID NO: 18), except for substitution of certain lysine amino acids naturally present in the wild-type sequence. For example, the novel intracellular domain comprises a sequence that is at least 70%, at least 75%, at least 80%, at least 85%, at least 90%, at least 95%, at least 96%, at least 97%, at least 98%, at least 98%, or is 100% identical to SEQ ID NO: 18, except for substitution of certain lysine amino acids naturally present in the wild-type sequence.

Thus, in some embodiments, the CAR, or any other protein that comprises a CD3-zeta intracellular domain, comprises a human CD3-zeta intracellular domain of SEQ ID NO: 18 except that each of at least six lysine amino acids of SEQ ID NO: 18 is substituted by an amino acid independently selected from the group consisting of alanine, aspartate, asparagine, glutamate, glutamine, histidine, leucine, methionine, serine, threonine, and valine.

In some embodiments, the CAR, or any other protein that comprises a CD3-zeta intracellular domain, comprises a human CD3-zeta intracellular domain of SEQ ID NO: 18 except that at least seven lysine amino acids of SEQ ID NO: 18 are substituted by an amino acid independently selected from the group consisting of alanine, aspartate, asparagine, glutamate, glutamine, histidine, leucine, methionine, serine, threonine, and valine.

In some embodiments, the CAR, or any other protein that comprises a CD3-zeta intracellular domain, comprises a human CD3-zeta intracellular domain of SEQ ID NO: 18 except that at least eight lysine amino acids of SEQ ID NO: 18 are substituted by an amino acid independently selected from the group consisting of alanine, aspartate, asparagine, glutamate, glutamine, histidine, leucine, methionine, serine, threonine, and valine.

In some embodiments, the CAR, or any other protein that comprises a CD3-zeta intracellular domain, comprises a human CD3-zeta intracellular domain of SEQ ID NO: 18 except that at least nine lysine amino acids of SEQ ID NO: 18 are substituted by an amino acid independently selected from the group consisting of alanine, aspartate, asparagine, glutamate, glutamine, histidine, leucine, methionine, serine, threonine, and valine.

In some embodiments, the CAR, or any other protein that comprises a CD3-zeta intracellular domain, comprises a human CD3-zeta intracellular domain of SEQ ID NO: 18 except that nine lysine amino acids of SEQ ID NO: 18 are substituted by an amino acid independently selected from the group consisting of alanine, aspartate, asparagine, glutamate, glutamine, histidine, leucine, methionine, serine, threonine, and valine.

In some embodiments where at least six, at least seven, at least eight, or at least nine lysine amino acids of SEQ ID NO: 18 are substituted, the substitutions can be selected from the group consisting of alanine, aspartate, and glutamate. In some embodiments where at least six, at least seven, at least eight, or at least nine lysine amino acids of SEQ ID NO: 18 are substituted, all of the substitutions can be with alanine. In some embodiments where at least six, at least seven, at least eight, or at least nine lysine amino acids of SEQ ID NO: 18 are substituted, all of the substitutions can be with aspartate. In some embodiments where at least six, at least seven, at least eight, or at least nine lysine amino acids of SEQ ID NO: 18 are substituted, all of the substitutions can be with glutamate.

The amino acid substitutions described above can be further understood by reference to sequences for certain embodiments of CARs of the present disclosure, or any other protein that comprises a CD3-zeta intracellular domain. Thus, in some embodiments, a CAR of the present disclosure, or any other protein that comprises a CD3-zeta intracellular domain, comprises a sequence (which corresponds to a modified CD3-zeta domain) selected from the group consisting of SEQ ID NOs: 2-5. In some embodiments, a CAR of the present disclosure, or any other protein that comprises a CD3-zeta intracellular domain, comprises a sequence (which corresponds to a modified CD3-zeta intracellular domain) selected from the group consisting of SEQ ID NOs: 19-66 and 68-86.

In some embodiments, a CAR of the present disclosure, or any other protein that comprises a CD3-zeta intracellular domain, comprises a human CD3-zeta intracellular domain of SEQ ID NO: 18 except that at least six lysine amino acids of SEQ ID NO: 18 are deleted. In some such embodiments, at least seven such lysine amino acids are deleted. In some such embodiments, at least eight such lysine amino acids are deleted. In some such embodiments, at least nine such lysine amino acids are deleted. In some such embodiments, nine such lysine amino acids are deleted.

In some embodiments, a CAR of the present disclosure, or any other protein that comprises a CD3-zeta intracellular domain, comprises a human CD3-zeta intracellular domain of SEQ ID NO: 18 except that at least six lysine amino acids of SEQ ID NO: 18 are substituted, in any of the manners described hereinabove, or are deleted. In some such embodiments, at least seven such lysine amino acids are substituted or deleted. In some such embodiments, at least eight such lysine amino acids are substituted or deleted. In some such embodiments, at least nine such lysine amino acids are substituted or deleted. In some such embodiments, nine such lysine amino acids are substituted or deleted.

Some of the intracellular domains for use in the present disclosure comprise, in addition to a CD3-zeta sequence of the present disclosure, or any other protein that comprises a CD3-zeta intracellular domain, a costimulatory domain that corresponds to a wild-type costimulatory domain, or wherein one or more lysine amino acids of the costimulatory domain have also been substituted or mutated, e.g., to alanine amino acids. See, e.g., Examples 4 and 5 and the costimulatory domain amino acid sequences of SEQ ID NOs: 57-60 and 62-66. Thus, in some embodiments, CARs of the present disclosure, or any other proteins that comprise a CD3-zeta intracellular domain, comprise a novel costimulatory domain wherein one, more than one, or all of the lysine amino acids that naturally occur in a costimulatory domain (or portion thereof, as incorporated into the CAR) are substituted by an amino acid independently selected from the group consisting of alanine, aspartate, asparagine, glutamate, glutamine, histidine, leucine, methionine, serine, threonine, and valine. In some embodiments, the substitutions can be selected from the group consisting of alanine, aspartate, and glutamate. In some embodiments, all of the substitutions are with alanine. In some embodiments, all of the substitutions are with aspartate. In some embodiments, all of the substitutions are with glutamate.

Example of proteins, at least portions of which can be incorporated into a CAR, in each case as a costimulatory domain, include MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIR2DS1, KIR2DS2, KIR3DS1, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83, and the like.

### Nucleic Acids and Vectors

In some embodiments, the present invention encompasses a nucleic acid molecule (e.g., DNA or RNA) that encodes a CAR of the present disclosure (e.g., any of the CARs provided herein), or any other protein that comprises a CD3-zeta intracellular domain. In some embodiments, the nucleic acid molecule is a DNA. In some embodiments, the nucleic acid molecule is an RNA. In some embodiments, the nucleic acid molecule comprises the sequence of a CAR, or any other protein that comprises a CD3-zeta intracellular domain, wherein the sequence comprises a nucleic acid sequence that encodes a CAR of the present disclosure, or any other protein that comprises a CD3-zeta intracellular domain.

Any of the nucleic acid molecules provided herein may include other features, for example a 5′ untranslated region (5′ UTR), a 3′ untranslated region (3′ UTR), a poly-adenine tail (polyA), a 7-methylguanosine cap (m⁷G), an internal ribosome entry site (IRES), and/or an open reading frame. In some embodiments, the disclosure provides an RNA having the following arrangement of features, or a DNA that encodes the same:
5′-[CAR]-3′
5′-[5′ UTR]-[CAR]-3′
5′-[m⁷G cap]-[5′ UTR]-[CAR]-3′
5′-[m⁷G cap]-[5′ UTR]-[CAR]-[polyA]-3′
5′-[CAR]-[polyA]-3′
5′-[CAR]-[3′ UTR]-[polyA]-3′
5′-[5′ UTR]-[CAR]-[3′ UTR]-3′
5′-[5′ UTR]-[CAR]-[3′ UTR]-[polyA]-3′
5′-[m⁷G cap]-[5′ UTR]-[CAR]-[3′ UTR]-[PolyA]-3′

Construction of nucleic acids to encode the CARs of the present disclosure can be further understood by reference to U.S. Pat. No. 10,934,337, which is incorporated herein by reference.

The present invention also provides vectors in which a DNA or RNA of the present invention is inserted. Construction of such vectors for the CARs of the present disclosure can be further understood by reference to U.S. Pat. No. 10,934,337, which is incorporated herein by reference.

### Cells Modified to Express the CARs

Any of the CARs of the present disclosure, or any other proteins of the present disclosure that comprise a CD3-zeta intracellular domain, presented herein can be expressed in a suitable cell. An example of a suitable cell is a T cell, which once modified to express the CAR is a CAR T cell. In some embodiments, the cell is a CD3+ cell. In some embodiments, the cell is a CD8+ cell. In some embodiments, the cell is a CD4+ cell. Other cells that can be suitable for CAR expression, or the expression of any other protein that comprises a CD3-zeta intracellular domain, include NK cells and stem cells, e.g., hematopoietic stem cells.

Methods of introducing and expressing genes into a cell are known in the art. In the context of an expression vector(s), the vector(s) can be readily introduced into a host cell, e.g., mammalian, bacterial, yeast, or insect cell by any method in the art. For example, the expression vector(s) can be transferred into a host cell by physical, chemical, or biological means. In some embodiments, the host cell is a T cell. Physical methods for introducing a polynucleotide into a host cell include electroporation, mechanical membrane disruption (e.g., cell squeezing or nanoparticle-based delivery), calcium phosphate precipitation, lipofection, particle bombardment, microinjection, and the like. Methods for producing cells comprising vectors and/or exogenous nucleic acids are well-known in the art. See, e.g., Sambrook et al. (2001, Molecular Cloning: A Laboratory Manual, Cold Spring Harbor Laboratory, New York). A preferred method for the introduction of a polynucleotide into a host cell is electroporation.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, e.g., U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). In the case where a non-viral delivery system is utilized, an exemplary delivery vehicle is a liposome. The use of lipid formulations is contemplated for the introduction of the nucleic acids into a host cell (in vitro, ex vivo or in vivo).

Regardless of the method used to introduce exogenous nucleic acids into a host cell or otherwise expose a cell to the inhibitor of the present invention, to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the present disclosure.

### RNA Transfection

In some embodiments, CAR T cells of the present disclosure, or other cells comprising a protein that comprises a modified CD3-zeta intracellular domain of the present disclosure, are obtained through the introduction of RNA (e.g., an mRNA comprises a sequence encoding a CAR as described herein). In some embodiments, an in vitro transcribed RNA CAR can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription can be a CAR of the present invention.

RNA can be introduced into target cells using any of a number of different methods, for instance, commercially available methods which include, but are not limited to, electroporation (Amaxa® Nucleofector-II® (Amaxa Biosystems, Cologne, Germany), ECM 830 (BTX) (Harvard Instruments, Boston, Mass.) Gene Pulser II® (BioRad, Denver, Colo.), Multiporator® (Eppendorf, Hamburg Germany), mechanical membrane disruption (e.g., cell squeezing, see U.S. Pat. Pub. No. 2014/287509A1), cationic liposome mediated transfection using lipofection, polymer encapsulation, peptide mediated transfection, or biolistic particle delivery systems such as “gene guns” (see, for example, Nishikawa, et al. Hum Gene Ther., 12 (8): 861-70 (2001).

Disclosed herein are methods for producing an in vitro transcribed RNA CAR, or in vitro transcribed RNA for any other protein that comprises a CD3-zeta intracellular domain. The present invention also includes a CAR encoding RNA construct, or an RNA construct of any other protein that comprises a CD3-zeta intracellular domain, that can be directly transfected into a cell. A method for generating mRNA for use in transfection can involve in vitro transcription (IVT) of a template with specially designed primers, followed by polyA addition, to produce a construct comprising 5′ and 3′ untranslated sequence (“UTR”), a 5′ cap, the nucleic acid to be expressed, and a polyA tail, typically 50-400, 50-2000 bases, 150-400 bases, or 150-2000 bases in length. RNA so produced can efficiently transfect different kinds of cells. In one aspect, the template includes sequences for the CARs of the present disclosure. In another aspects, the template includes sequences for the other proteins of the present disclosure that comprise a CD3-zeta intracellular domain.

The production of mRNA, for example, by in vitro transcription (IVT) from a DNA template, is known in the art. For example, one method for generating mRNA for use in transfection involves in vitro transcription (IVT) of a template with specially designed primers. Optionally, the mRNA is 3′ polyadenylated by methods known in the art and may comprise, for example, a 3′ polyadenine tail of about 25, 50, 100, 150, 250, 500, or 1000 adenine nucleotides.

In some embodiments, the nucleic acid is a self-amplifying RNA (saRNA) prepared according to methods known in the art. In some embodiments, the RNA, e.g., mRNA, comprises pseudouridine. In some embodiments, the RNA is artificially enriched in pseudouridine. In some embodiments, substantially all the uridine nucleotides (e.g., greater than 90%, 95%, 97%, 99% or 99.9%) of the RNA are substituted with pseudouridine. Methods for incorporating pseudouridine into an RNA are known in the art.

In some embodiments, the nucleic acid is circular RNA prepared according to methods known in the art.

### Therapeutic Applications

In some embodiments, the present invention encompasses a cell (e.g., T cell) modified to express an CAR of the present disclosure, or other protein of the present disclosure that comprises a CD3-zeta intracellular domain. Therefore, in some instances, the transduced immune cell (e.g., T cell) can elicit a CAR-mediated immune (e.g., T-cell) response, cytotoxic response, or anti-tumor response. In some embodiments, the present disclosure provides the use of a CAR of the present disclosure to redirect the specificity of a primary T cell to a surface marker or tumor antigen. Thus, in some embodiments, the present invention also provides a method for stimulating a T cell-mediated cytotoxic or immune response to a target cell population or tissue in a mammal comprising the step of administering to the mammal a T cell that expresses a CAR of the present disclosure, wherein the CAR comprises an antigen-binding domain that specifically binds a predetermined target or surface marker (e.g., BCMA). In some embodiments, the present invention includes a type of cellular therapy where T cells are genetically modified to express a CAR of the present disclosure and the CAR T cell is infused to a recipient in need thereof. The infused cell is able to kill target cells in the recipient. Unlike antibody therapies, some CAR T cells are able to replicate in vivo resulting in long-term persistence of such cells.

The CAR-modified T cells of the present disclosure, or the cells modified with a protein of the present disclosure that comprises a CD3-zeta intracellular domain, may also serve as a type of vaccine for ex vivo immunization and/or in vivo therapy in a mammal. Preferably, the mammal is a human.

With respect to ex vivo cell production, at least one of the following occurs in vitro prior to administering the cell into a mammal: i) expansion of the cells, ii) introducing a nucleic acid encoding a CAR to the cells, and/or iii) cryopreservation of the cells. Ex vivo procedures are well known in the art and are discussed more fully below. Briefly, cells are isolated from a mammal (e.g., a human) and genetically modified (e.g., transduced or transfected in vitro) with a nucleic acid or vector expressing a CAR of the present disclosure as disclosed herein. The CAR-modified cell can be administered to a mammalian recipient to provide a therapeutic benefit. The mammalian recipient may be a human and the CAR-modified cell can be autologous with respect to the recipient. Alternatively, the cells can be allogeneic, syngeneic, or xenogeneic with respect to the recipient.

The CAR-modified immune cells (e.g., CAR T cells) of the present invention, or a composition comprising such cells, may be used, or may be administered to a subject in need thereof, in an effective amount, to provide anti-tumor immunity; to treat or prevent cancer; to treat or prevent autoimmune condition; or to treat or prevent an allergic condition. In some embodiments, the cancer is multiple myeloma, Hodgkin lymphoma, non-Hodgkin lymphoma, a leukemia, or glioblastoma. In some embodiments, the autoimmune condition is myasthenia gravis, systemic lupus erythematosus, rheumatoid arthritis, pemphigus, psoriasis, inflammatory bowel disease, celiac sprue, pernicious anemia, idiopathic thrombocytopenia purpura, scleroderma, Graves disease, Sjögren syndrome, Goodpasture syndrome, or type 1 diabetes. In some embodiments, the allergic condition is anaphylaxis, asthma, food allergy, stinging insect allergy, drug allergy, allergic rhinitis, urticaria, angioedema, eczema, atopic dermatitis, contact dermatitis, and eosinophilic esophagitis.

The CAR-modified immune cells (e.g., CAR T cells) of the present invention may be administered either alone, or as a composition (e.g., a pharmaceutical composition) in combination with diluents and/or with other components such as IL-2 or other cytokines or cell populations. Briefly, pharmaceutical compositions of the present invention may comprise a target cell population as described herein, in combination with one or more pharmaceutically or physiologically acceptable carriers, diluents, or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Compositions of the present invention are preferably formulated for intravenous administration but can be formulated for other routes of parenteral administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount,” “an anti-tumor effective amount,” “a tumor-inhibiting effective amount,” or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the CAR-modified immune cells (e.g., CAR T cells) of the present disclosure as described herein may be administered at a dosage of 10⁴ to 10⁹ cells/kg body weight, preferably 10⁵ to 10⁹ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting treatment accordingly.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the present disclosure described herein may be more fully understood, the following examples are set forth. The synthetic examples described in this application are offered to illustrate the compounds and methods provided herein and are not to be construed in any way as limiting their scope.

Unless defined otherwise, all technical and scientific terms used herein have the same meaning as commonly understood by one of ordinary skill in the art to which the present disclosure pertains. Although any methods and materials similar or equivalent to those described herein can be used in the practice for testing of the present invention, the preferred materials and methods are described herein. In describing and claiming the present invention, the following terminology will be used.

### Example 1

CAR T cells were produced by use of an mRNA construct of the present disclosure that encoded a CAR protein of the present disclosure comprising a mutated CD3-zeta intracellular domain to prevent downregulation of the protein. A series of experiments were then performed on them. The CAR proteins investigated either comprised a wild-type CD3-zeta intracellular domain sequence (SEQ ID NO: 1), a mutated sequence with all CD3-zeta intracellular domain lysines mutated to arginine (SEQ ID NO: 2), a mutated sequence with all CD3-zeta intracellular domain lysines mutated to alanine (SEQ ID NO: 3), a mutated sequence with all CD3-zeta intracellular domain lysines mutated to aspartate (SEQ ID NO: 4), or a mutated sequence with all CD3-zeta intracellular domain lysines mutated to glutamate (SEQ ID NO: 5). The CAR T cells were observed to express high levels of CAR protein, bind BCMA, and kill BCMA+ tumor cells. Exposure of the CAR T cells to target cells expressing the BCMA ligand led to low levels of cell surface CAR protein downregulation.

mRNA constructs of the present disclosure, separately comprising the nucleotide sequence of SEQ ID NOs: 13-17, were generated by in vitro transcription from a PCR-amplified DNA template. In vitro transcription was performed using a T7 RNA polymerase and a PCR-product template that included a 180-nucleotide poly A tail. A 7-methylguanosine cap (CleanCap) was incorporated at the 5′ end of the mRNA during the co-transcriptional mRNA synthesis.

An mRNA construct of the present disclosure comprised SEQ ID NO: 13, and comprised, from 5′ to 3′: a 5′ cap, a 5′ UTR described as SEQ ID NO: 11, an open reading frame (ORF) described as SEQ ID NO: 6, a 3′ UTR described as SEQ ID NO: 12, and a 3′ polyadenine tail of 150 adenine units or more. The ORF encoded a CAR protein of the present disclosure with the amino acid sequences of SEQ ID NO: 1.

Another mRNA construct of the present disclosure comprised SEQ ID NO: 14, and comprised, from 5′ to 3′: a 5′ cap, a 5′ UTR described as SEQ ID NO: 11, an open reading frame (ORF) described as SEQ ID NO: 7, a 3′ UTR described as SEQ ID NO: 12, and a 3′ polyadenine tail of 150 adenine units or more. The ORF encoded a CAR protein of the present disclosure with the amino acid sequences of SEQ ID NO: 2.

Another mRNA construct of the present disclosure comprised SEQ ID NO: 15 and comprised, from 5′ to 3′: a 5′ cap, a 5′ UTR described as SEQ ID NO: 11, an open reading frame (ORF) described as SEQ ID NO: 8, a 3′ UTR described as SEQ ID NO: 12, and a 3′ polyadenine tail of 150 adenine units or more. The ORF encoded a CAR protein of the present disclosure with the amino acid sequences of SEQ ID NO: 3.

Another mRNA construct of the present disclosure comprised SEQ ID NO: 16 and comprised, from 5′ to 3′: a 5′ cap, a 5′ UTR described as SEQ ID NO: 11, an open reading frame (ORF) described as SEQ ID NO: 9, a 3′ UTR described as SEQ ID NO: 12, and a 3′ polyadenine tail of 150 adenine units or more. The ORF encoded a CAR protein of the present disclosure with the amino acid sequences of SEQ ID NO: 4.

Another mRNA construct of the present disclosure comprised SEQ ID NO: 17 and comprised, from 5′ to 3′: a 5′ cap, a 5′ UTR described as SEQ ID NO: 11, an open reading frame (ORF) described as SEQ ID NO: 10, a 3′ UTR described as SEQ ID NO: 12, and a 3′ polyadenine tail of 150 adenine units or more. The ORF encoded a CAR protein of the present disclosure with the amino acid sequences of SEQ ID NO: 5.

To prepare CAR T cells from the mRNA constructs, lymphocytes were obtained from whole blood of a healthy human donor. From these lymphocytes, CD8+ T cells were positively selected by use of paramagnetic microbeads conjugated to an anti-CD8 antibody. This yielded cells that were 95% CD8+ T cells and 95% viable. These enriched CD8+T cells were expanded by incubation at 37° C. with 5% CO₂ in the presence of anti-CD3 antibody (clone OKT3), IL-7, and IL-15 for up to 14 days. The cells were transfected with 0.1 μg/μl of the mRNA construct by electroporation (4D Nucleofector, Lonza) according to manufacturer's instructions. Cells were then returned to culture in complete medium containing IL-7 and IL-15 overnight.

CAR T cells obtained from the above-described process were tested for viability, CAR protein expression, BCMA binding, cytotoxicity, i.e., the ability to kill BCMA+ myeloma (tumor) cells, and cytokine production. Resistance of the CAR protein to downregulation was tested by incubation of CAR T cells with, or without, BCMA+ myeloma cells, followed by analysis of CAR expression. Viability, CAR expression, and BCMA binding were determined by flow cytometry on a Guava® EasyCyte® 12HT Flow cytometer (Luminex). To test viability, a sample of the CAR T cells was mixed with propidium iodide and acridine orange and analyzed by fluorescence microscopy using a Nexcelom Auto 2000 cytometer. To test CAR protein expression and BCMA binding, a sample of the CAR T cells was incubated with 0.4 μg/mL of allophycocyanin (APC)-conjugated BCMA (Recombinant Human TNFRSF17 protein, Fc-tagged, APC labeled; Creative Biomart, Shirley, NY). CAR expression was assessed on the flow cytometer with electronic gating on fluorescence in the red channel to detect presence or absence of emission from BCMA-APC on CAR-positive and CAR-negative cells. BCMA binding was determined by measuring the intensity of fluorescence in the red channel to determine the relative quantity of BCMA-APC bound to labelled CAR-positive cells. For the viability, expression, cytotoxicity, cytokine production and BCMA binding assays, control (non-CAR) CD8+ T cells generated by electroporation without IVT mRNA were tested as a parallel control.

To test the capacity of the wild-type and mutated CAR proteins (SEQ ID NOs: 1-5) of the present disclosure to resist antigen-mediated downregulation, 50,000 CAR T cells generated with mRNA sequence (SEO ID NOs: 13-17) of the present disclosure co-incubated with 100,000 MM1S tumor cells in wells of a 96-well V-bottom plate. The MM1S tumor cell line is derived from a patient with IgA Lambda Multiple Myeloma. See, e.g., Greenstein et al., Exp. Hematol (2003) 31(4): 271-82 (DOI=10.1016/S0301-472X(03)00023-7). Additional cultures of CAR T cells alone (without target cells) were prepared as a control. Following 24 hours of incubation at 37° C., tissue culture supernatants and cells were harvested. CAR expression was analyzed by staining cells with CD8-BV421 antibody, propidium iodide and BCMA-APC. Viable CD8+ T cells were identified by exclusion of dead cells staining with propidium iodide (near-infrared fluorescence) and selection of CD8+ cells with blue fluorescence off the violet laser. CAR expression on total viable CD8+ T cells was quantitated by intensity of red fluorescence off the red laser. Signaling of the CAR was evaluated by analysis of Interferon-gamma production in the tissue culture supernatant by specific ELISA.

Capacity of the remaining expressed CAR protein to signal was determined by a secondary culture. The secondary culture was set up by co-culturing pre-exposed CAR T cells with MM1S-GFP tumor cells. Aliquots of 50,000 MM1S-GFP tumor cells were placed in wells of a 96-well plate. CAR T cells from the primary cell culture were washed to remove residual components of the media. Between about 1,500 to 50,000 washed CAR T cells were added to each well to obtain various effector:target ratios (i.e., ratios of CAR T cells to BCMA+ myeloma cells) that were between 1:1 and 1:32. Following 24-72 hours of incubation, propidium iodide was used to stain dead cells. Viable target cells were identified by expression of GFP (green fluorescence off the blue laser) and exclusion of propidium iodide, and cell density was determined by flow cytometry. The degree of myeloma cell killing by the CAR T cells was calculated by comparison to the number of myeloma cells in wells concurrent control wells that did not contain CAR T cells. Signaling was evaluated by analysis of Interferon-gamma production in the supernatant using specific ELISA.

The CAR T cells of the present disclosure showed similar viability following electroporation with all constructs of the present disclosure (comprising sequences of SEQ ID NOs: 13-17). The percentage of CAR T cells expressing anti-BCMA CAR was similar with all constructs (91.3 to 94.0%).

TABLE 1
CAR T Cell Viability and CAR Expression
following electroporation with IVT mRNA

| Relevant | CD3-zeta | | |
|---|---|---|---|
| construct | intracellular | Viability | Expression of anti- |
| (SEQ ID NO) | domain | (%) | BCMA CAR (%) |
| 13 | wild-type | 76.8 ± 5.3 | 91.3 ± 4.0 |
| 14 | K-R | 67.6 ± 5.8 | 94.0 ± 1.7 |
| 15 | K-A | 61.7 ± 11.5 | 93.7 ± 1.5 |
| 16 | K-D | 73.8 ± 3.5 | 91.3 ± 3.1 |
| 17 | K-E | 79.5 ± 3.0 | 93.3 ± 2.9 |

Mean ± SD (n = 3)

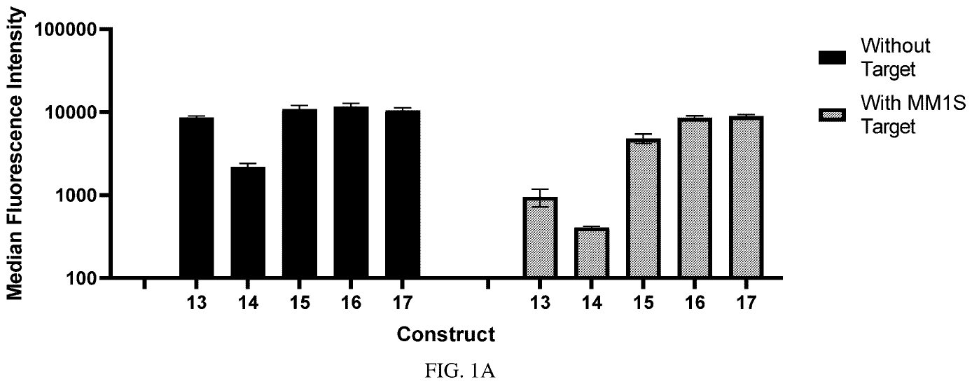

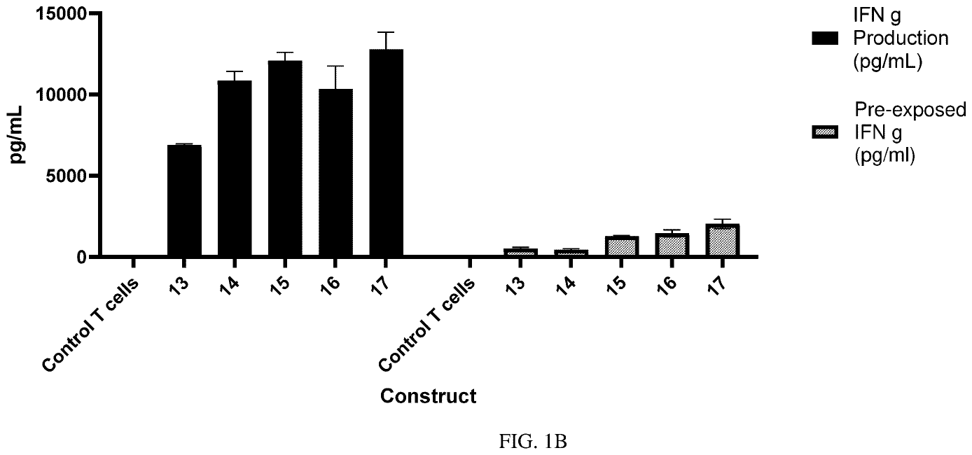

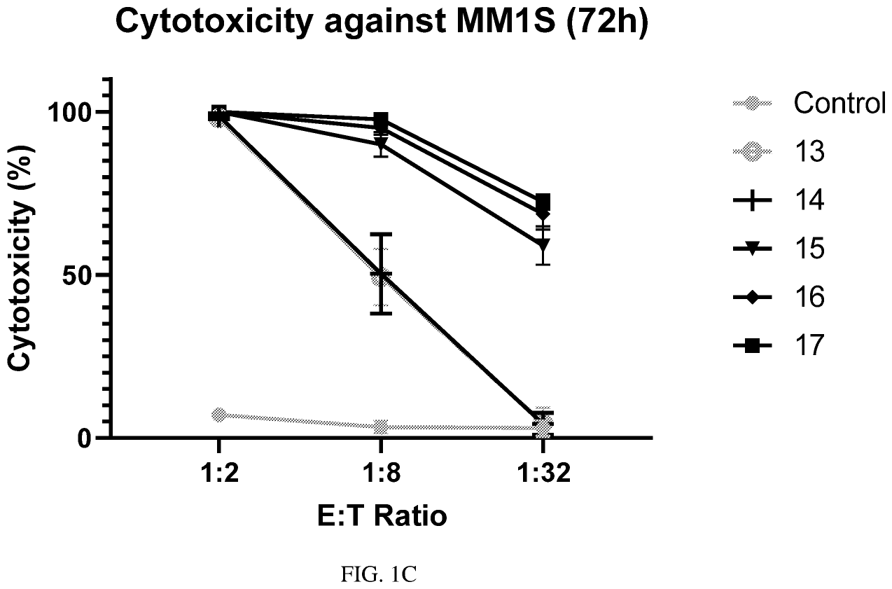

Exposure of CAR T cells of the present disclosure to the ligand (target), BCMA, on MM1S myeloma target cells for 24 hours led to downregulation of CAR expression. CAR T cells generated with a construct (SEQ ID NO: 13) that encoded a protein that comprised the wild-type CD3-zeta intracellular domain sequence showed a downregulation in CAR expression from 8,633 Median Fluorescence Intensity (MFI) in the absence of BCMA ligand to 950 MFI in the presence of BCMA ligand expressed by MM1S myeloma target cells (i.e., a 89% drop in CAR expression) (FIG. 1A). Construct SEQ ID NO: 14 was designed to encode a protein that had mutations of all CD3-zeta intracellular domain lysine residues to arginine. Generation of the CAR T cells with that construct (SEQ ID NO: 14) produced cells with lower expression of the anti-BCMA CAR than the wild-type construct SEQ ID NO: 13 (2,188 MFI compared with 8,633 MFI). Exposure of these CAR T cells to target ligand BCMA on MM1S target cells led to further reduction in surface CAR expression (405 MFI with SEQ ID NO: 14 compared with 950 MFI with the wild-type construct SEQ ID NO: 13) (FIG. 1A).

Other amino acid substitutions were introduced in place of the CAR CD3-zeta intracellular domain lysines. IVT mRNA of SEQ ID NO: 15 encodes a protein having all CD3-zeta intracellular lysines mutated to alanine. IVT mRNA SEQ ID NO: 16 encodes a protein having all CD3-zeta intracellular lysines mutated to aspartate. IVT mRNA SEQ ID NO: 17 encodes a protein having all CD3-zeta intracellular lysines mutated to glutamate. Generation of CAR T cells with these constructs (SEQ ID NOs: 15, 16, 17) led to CAR T cells with remarkably greater CAR expression and enhanced resistance to ligand-mediated CAR downregulation. CAR T cells generated with these constructs exhibited higher CAR expression than wild-type CAR (10,493 to 11,678 MFI compared with 8,633 from the wild-type construct). Downregulation was dramatically reduced with each of SEQ ID NOs: 15, 16, and 17, with 44.2%, 73.0% and 85.3% of CAR expression after exposure to BCMA ligand on MM1S myeloma cells, respectively, compared with the 11.0% of CAR expression maintained by wild-type construct SEQ ID NO: 13 (FIG. 1A).

TABLE 2
Expression of anti-BCMA CAR by CAR T cells following culture
in the absence or presence of MM1S target cells

| | | | CAR expression | CAR expression |
|---|---|---|---|---|
| Construct | CD3-zeta | CAR expression in | following exposure to | maintained |
| (SEQ ID | intracellular | absence of target | BCMA + MM1S cells | following ligand |
| NO) | domain | ligand (MFI) | (MFI) | exposure (%) |
| 13 | wild-type | 8633 ± 638 | 950 ± 393 | 11.0% |
| 14 | K-R | 2188 ± 378 | 405 ± 25 | 18.5% |
| 15 | K-A | 10892 ± 2021 | 4810 ± 1108 | 44.2% |
| 16 | K-D | 11678 ± 1922 | 8526 ± 850 | 73.0% |
| 17 | K-E | 10493 ± 1365 | 8947 ± 672 | 85.3% |

Mean ± SD (n = 3)

CAR T cells of the present disclosure generated with CD3-zeta intracellular domain lysines mutated to arginine, alanine, aspartate and glutamate (SEQ ID NOs: 14-17) all maintained capacity to signal. Supernatants of co-cultures between the CAR T cells of the present disclosure and BCMA⁺ MM1S multiple myeloma were analyzed for expression of Interferon-gamma by specific ELISA. Prior to co-culture, CAR T cells were pre-exposed or not pre-exposed to BCMA+ cells. All constructs of the present disclosure showed high levels of Interferon-gamma production. Expression of Interferon-gamma was comparable between CAR T generated with lysine-mutant constructs SEQ ID NOs: 14-17 (10,326 to 12,767 pg/mL). CAR T cells generated with lysine-mutant constructs showed higher Interferon-gamma expression than wild-type CAR T cells (SEQ ID NO: 13; 6,881 pg/mL). Therefore, mutation of the CAR CD3-zeta intracellular domain lysines to other amino acids including arginine, alanine, aspartate, and glutamate permits, and does not interfere with, CAR T cell activation (FIG. 1B).

TABLE 3
Cytokine (Interferon-gamma) production by anti-BCMA CAR
T cells cultured in the presence of MM1S target cells

| | CD3-zeta intracellular | |
|---|---|---|
| Construct | domain | Interferon-gamma (pg/mL) |
| Control T cells | N/A | not detected |
| SEQ ID NO: 13 | wild-type | 6881 ± 131 |
| SEQ ID NO: 14 | K-R | 10845 ± 1001 |
| SEQ ID NO: 15 | K-A | 12065 ± 922 |
| SEQ ID NO: 16 | K-D | 10326 ± 2484 |
| SEQ ID NO: 17 | K-E | 12767 ± 1842 |

Mean ± SD (n = 3);
N/A = not applicable

CAR T cells generated with SEQ ID NOs: 15-17 maintained higher expression of CAR following exposure to the BCMA ligand than wild-type (SEQ ID NO: 13). The superiority of these constructs was tested in a secondary culture. Pre-exposed CAR T cells generated with constructs of the present disclosure encoding lysine-mutant CAR and wild-type CAR were used to evaluate cytotoxicity and cytokine production against MM1S-GFP target cells expressing BCMA ligand. Cytotoxicity was evaluated by co-culture at various effector:target ratios. At a 1:2 effector:target ratio, control T cells showed no cytotoxicity against the MM1S-GFP target cell (<10%), while pre-exposed CAR T cells generated with each of SEQ ID NOs: 13-17 showed total or near-total elimination of target cells (98-100% cytotoxicity) (FIG. 1C). At lower effector:target cell ratios, the wild-type pre-exposed CAR-T cells (SEQ ID NO: 13) showed dramatic reduction in cytotoxicity (49.3% at 1:8 and 4.7% at 1:32) (FIG. 1C). CAR T cells generated with IVT mRNA for SEQ ID NO: 14, encoding a protein with the lysine-to-arginine mutant, showed a similar cytotoxicity profile (FIG. 1C). In contrast, pre-exposed CAR T cells generated with IVT mRNA for each of SEQ ID NOs: 15, 16, and 17 showed high levels of cytotoxicity down to a 1:32 ratio (59.0%, 68.7% and 72.3%, respectively) (FIG. 1C). This maintenance of cytolytic activity at very low effector:target ratios was matched by higher levels of interferon-gamma production (at a 1:2 ratio) by pre-exposed CAR T cells generated with these constructs (Table 4 and FIG. 1B, right). Pre-exposed CAR T cells generated with constructs in which the intracellular domain lysines were mutated to amino acids with opposing negative charge—aspartate (SEQ ID NO: 16) and glutamate (SEQ ID NO: 17) —showed the highest cytotoxic activity and cytokine production.

TABLE 4
CAR T cell 72-hour cytotoxicity and 24-hour cytokine production
activity following exposure to BCMA ligand (MM1S)

| | | | | | Interferon- |
|---|---|---|---|---|---|
| | CD3-zeta | % MM1S-GFP | % MM1S-GFP | % MM1S-GFP | gamma |
| | intracellular | Cytotoxicity | Cytotoxicity | Cytotoxicity | production |
| Construct | domain | (1:2) | (1:8) | (1:32) | (pg/mL) |
| Control T cells | N/A | 7.1 ± 2.1 | 3.4 ± 3.2 | 3.0 ± 4.1 | 0 ± 0 |
| SEQ ID NO: 13 | wild-type | 98.0 ± 2.6 | 49.3 ± 15.0 | 4.7 ± 8.0 | 519 ± 132 |
| SEQ ID NO: 14 | K-R | 98.7 ± 1.5 | 50.3 ± 21.0 | 4.3 ± 5.9 | 442 ± 102 |
| SEQ ID NO: 15 | K-A | 100 ± 0.0 | 90.0 ± 6.6 | 59.0 ± 10.1 | 1267 ± 94 |
| SEQ ID NO: 16 | K-D | 100 ± 0.0 | 95.0 ± 3.5 | 68.7 ± 8.1 | 1449 ± 372 |
| SEQ ID NO: 17 | K-E | 100 ± 0.0 | 97.7 ± 1.2 | 72.3 ± 4.0 | 2026 ± 509 |

mean ± SD (n = 3)

Thus, CAR T cells of the present disclosure generated with an IVT mRNA encoding a CAR of the present disclosure (SEQ ID NOs: 14, 15, 16, and 17) provide superior cytotoxicity and cytokine production compared with wild-type CAR T constructs.

### Example 2

A series of experiments was performed to test how variation in the number of CAR CD3-zeta intracellular domain lysine residues that were mutated to alanine residues would affect resistance of the CAR to downregulation by target binding.

Numerous IVT mRNA constructs were prepared that encoded variations of an anti-BCMA CAR protein that were identical with the exception of the intracellular polypeptide sequence encoding the signaling domains. The constructs encoding CARs that comprised either wild-type CD3-zeta intracellular domain sequence (SEQ ID NO: 18), CD3-zeta intracellular domain sequence with one single lysine mutated to alanine (SEQ ID NOs: 19-27), CD3-zeta intracellular domain sequence with between 2 and 7 lysine residues mutated to alanine (SEQ ID NOs: 28-33), CD3-zeta intracellular domain sequence with all but one (i.e., 8) of the lysine residues mutated to alanine (SEQ ID NO: 34-42) or a CD3-zeta intracellular domain sequence with all nine lysine residues mutated to alanine (SEQ ID NO: 43). These constructs were otherwise identical with respect to the Cap, 5′ UTR, open reading frame encoding the signal peptide, scFv and transmembrane sequences, 3′ UTR and poly A tail. The contribution of either individual lysines or the ensemble of the lysine amino acids to downregulation of CAR following exposure to CAR ligand (BCMA) was investigated.

For each of the mRNA constructs, CAR T cells were prepared substantially as described in Example 1. 24 hours after transfection, CAR T cells made from each mRNA construct were exposed to MM1S or no target cells by the method described in Example 1. 24 hours later, the cells were evaluated for cell count, viability, maintenance of CAR expression, and cytokine production by the method described in Example 1. The constructs and their effect on downregulation of CAR expression are shown below.

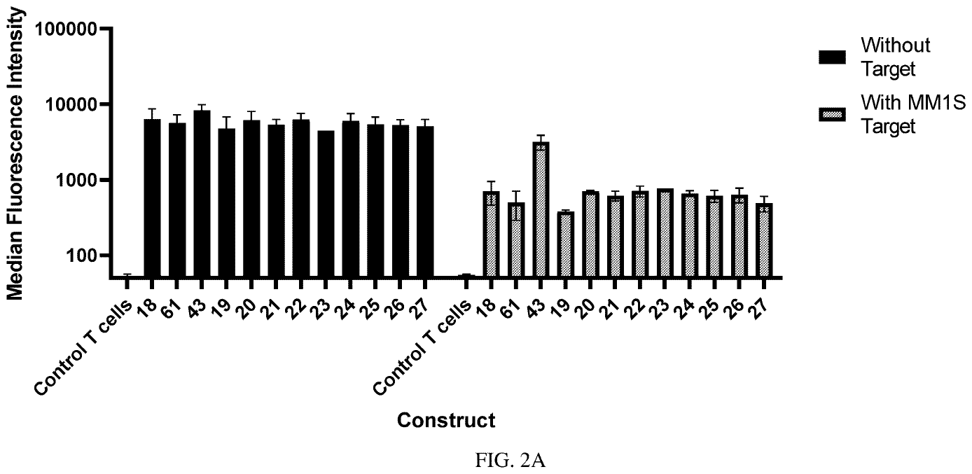

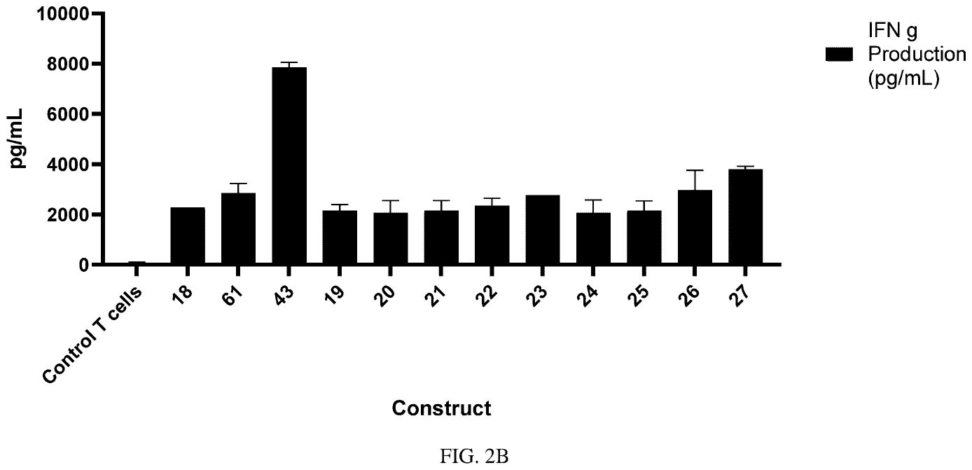

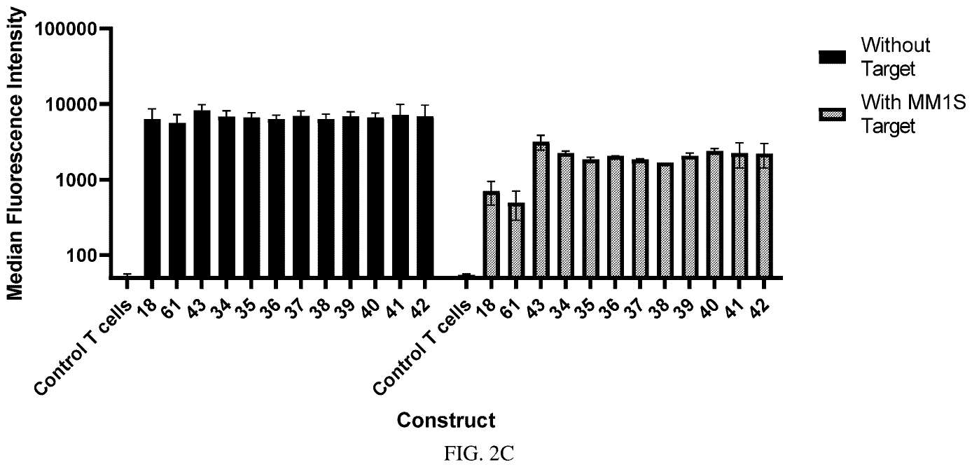

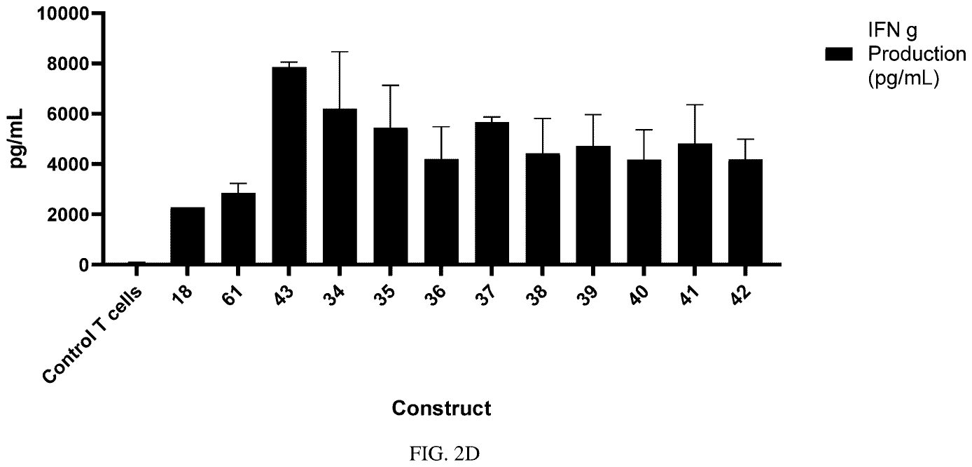

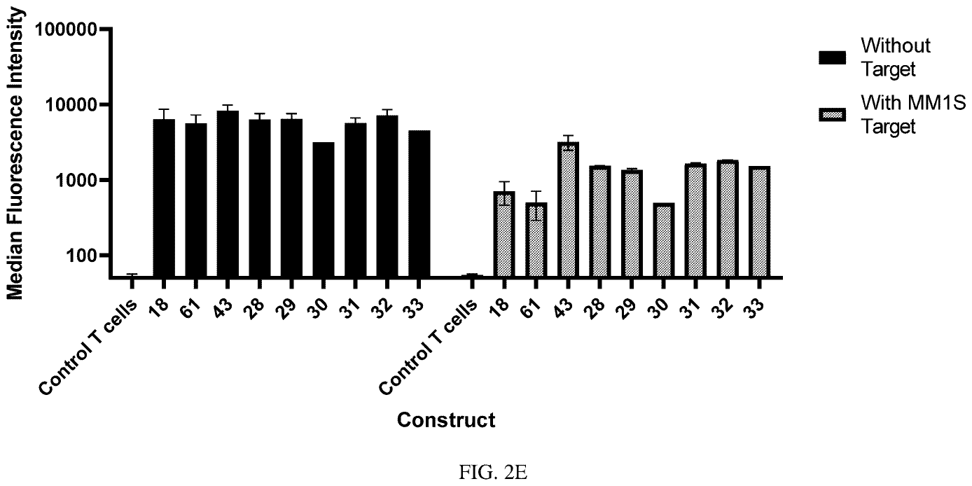

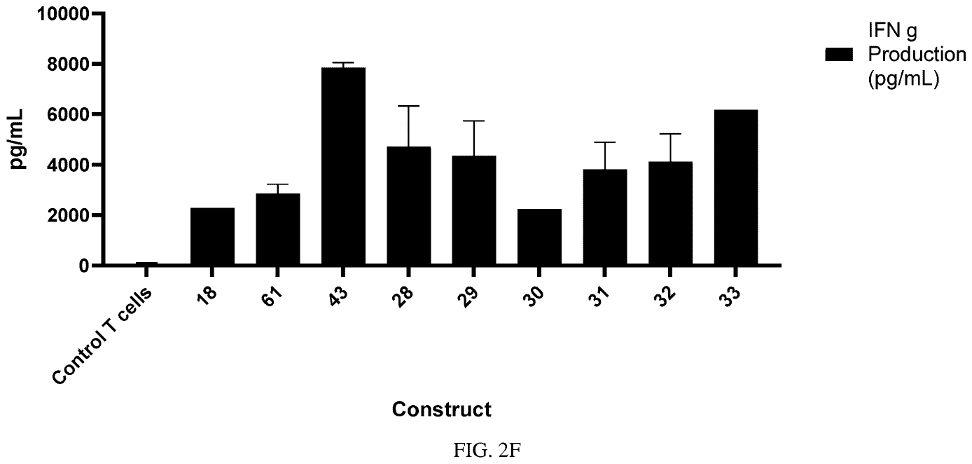

CAR T cells generated with all test constructs showed good viability following electroporation (viability >70%). All CAR T cells showed a high percentage of anti-BCMA CAR expression following electroporation (>85%). Exposure of CAR T cells to the ligand, BCMA, on MM1S myeloma target cells for 24 hours led to downregulation of CAR expression. CAR T cells generated with IVT mRNA comprising wild-type intracellular polypeptide sequence (SEQ ID NO: 18) showed a downregulation of CAR expression from 6,358 MFI to 705 MFI, i.e., a reduction in CAR expression of 89%. In contrast, CAR T cells generated using IVT mRNA in which all 9 intracellular lysines were mutated to alanine (SEQ ID NO: 43) showed a downregulation in CAR expression from 8,264 MFI to 3,165 MFI, with 38% of CAR expression maintained after exposure to the BCMA ligand. CAR T cells generated with constructs comprising individual CD3-zeta intracellular lysine-alanine mutations (SEQ ID NOs: 19-27) showed downregulation of CAR expression from 5,707±609 MFI in the absence of ligand to 617±119 MFI in the presence of BCMA+ MM1S myeloma cells (Mean±SD for all constructs) (FIG. 2A). This reduction in anti-BCMA CAR expression represents an 89%±3% drop in CAR expression and was therefore comparable to the 89% drop in CAR expression observed with the wild-type construct (SEQ ID NO: 18) (FIG. 2A). CAR T cells generated with constructs comprising CD3-zeta intracellular lysine-alanine mutations at 8 of 9 lysines (SEQ ID NOs: 34-42) showed downregulation in CAR expression from 6,736±299 MFI in the absence of ligand to 2,064±234 MFI in the presence of BCMA+ MM1S myeloma cells (mean±SD for all constructs) (FIG. 2C). Following exposure to BCMA ligand, the anti-BCMA CAR expression remained at 31%±3% of the CAR expression observed in the absence of ligand, showing a pattern of expression similar to CAR cells generated with an IVT mRNA that has all CD3-zeta intracellular domain lysines mutated to alanine (SEQ ID NO: 43) (FIG. 2A). CAR T cells generated with IVT mRNA constructs comprising CD3-zeta intracellular lysine-alanine mutations at 2-7 lysines (SEQ ID NOs: 28-33) showed intermediate effects, with 25%+6% of the anti-BCMA CAR MFI remaining after exposure to BCMA ligand compared with culture in the absence of ligand (FIG. 2E). Similar trends were observed with interferon-gamma production from CAR T cells generated with constructs comprising CD3-zeta intracellular domains that had various numbers of lysine-alanine mutations. CAR T cells generated with the wild-type IVT mRNA construct (SEQ ID NO: 18) produced 2,291 pg/mL of interferon-gamma following co-culture with MM1S (FIG. 2B). CAR T cells generated with the 9 lysine-alanine mutant construct (SEQ ID NO: 43) produced 7,853±285 pg/mL of interferon-gamma upon co-culture (FIG. 2B). Similar to wild-type IVT mRNA (SEQ ID NO: 18), the CAR T cells generated with constructs comprising CD3-zeta intracellular domains with one lysine-alanine mutation (SEQ ID NO: 19-27) produced 2,498±583 pg/mL of interferon-gamma during co-culture with MM1S (FIG. 2B). CAR T cells generated with constructs comprising CD3-zeta intracellular domains with 8 lysine-alanine mutations (SEQ ID NOs: 34-42) produced 4,871±739 pg/mL of interferon-gamma during co-culture with MM1S, a level that was higher than the wild-type construct but reduced compared with the fully-mutated construct (SEO) ID NO: 43) (FIG. 2D). Finally, the constructs with intermediate numbers of 2-7 lysine-alanine mutations (SEQ ID NO: 28-33) generated CAR T cells that produced, on average, 4,241±1,279 pg/mL of interferon-gamma upon culture with MM1S, representing an intermediate expression level compared with the wild-type (SEQ ID NO: 18) and fully-mutated (SEQ ID NO: 43) constructs (FIG. 2F).

TABLE 5
Expression of anti-BCMA CAR by CAR T cells expressing CAR comprising
CD3-zeta intracellular domains with lysine-alanine mutations following
culture in the absence or presence of MM1S target cells

| | | CAR | CAR expression | CAR |
|---|---|---|---|---|
| Construct CD3- | | expression | after exposure to | expression |
| zeta intracellular | Number of K- | without target | BCMA + MM1S | after target |
| domain | A mutations | (MFI) | cells (MFI) | exposure (%) |
| SEQ ID NO: 18 | 0 | 6358 ± 3216 | 705 ± 346 | 11% |
| SEQ ID NO: 43 | 9 | 8264 ± 3071 | 3165 ± 1427 | 38% |
| SEQ ID NO: 19 | 1 | 4755 ± 2871 | 380 ± 23 | 8% |
| SEQ ID NO: 20 | 1 | 6131 ± 2662 | 703 ± 24 | 11% |
| SEQ ID NO: 21 | 1 | 5330 ± 1329 | 614 ± 129 | 12% |
| SEQ ID NO: 22 | 1 | 6211 ± 1917 | 708 ± 165 | 11% |
| SEQ ID NO: 23 | 1 | 4437 | 766 | 17% |
| SEQ ID NO: 24 | 1 | 5981 ± 2145 | 656 ± 79 | 11% |
| SEQ ID NO: 25 | 1 | 5390 ± 1916 | 611 ± 156 | 11% |
| SEQ ID NO: 26 | 1 | 5318 ± 1292 | 634 ± 197 | 12% |
| SEQ ID NO: 27 | 1 | 5117 ± 1605 | 486 ± 160 | 9% |
| SEQ ID NO: 34 | 8 | 6840 ± 1882 | 2263 ± 170 | 33% |
| SEQ ID NO: 35 | 8 | 6629 ± 1483 | 1847 ± 184 | 28% |
| SEQ ID NO: 36 | 8 | 6323 ± 1189 | 2057 ± 21 | 33% |
| SEQ ID NO: 37 | 8 | 6954 ± 1667 | 1844 ± 58 | 27% |
| SEQ ID NO: 38 | 8 | 6282 ± 1481 | 1674 ± 3 | 27% |
| SEQ ID NO: 39 | 8 | 6890 ± 1463 | 2062 ± 254 | 30% |
| SEQ ID NO: 40 | 8 | 6610 ± 1395 | 2376 ± 277 | 36% |
| SEQ ID NO: 41 | 8 | 7178 ± 3922 | 2245 ± 1166 | 31% |
| SEQ ID NO: 42 | 8 | 6917 ± 3948 | 2211 ± 1116 | 32% |
| SEQ ID NO: 28 | 7 | 6249 ± 1875 | 1531 ± 13 | 24% |
| SEQ ID NO: 29 | 6 | 6387 ± 1667 | 1352 ± 68 | 21% |
| SEQ ID NO: 30 | 2 | 3145 | 494 | 16% |
| SEQ ID NO: 31 | 7 | 5650 ± 1382 | 1642 ± 49 | 29% |
| SEQ ID NO: 32 | 7 | 7111 ± 2007 | 1833 ± 21 | 26% |
| SEQ ID NO: 33 | 7 | 4516 | 1524 | 34% |

TABLE 6
Interferon-gamma production by anti-BCMA CAR T cells expressing
CAR comprising CD3-zeta intracellular domain lysine-alanine
mutations in presence of MM1S target cells

| Construct CD3-zeta | Number of K-A mutations in | Interferon-gamma |
|---|---|---|
| intracellular domain | intracellular domain | (pg/mL) |
| Control T cells | N/A | 52 |
| SEQ ID NO: 18 | 0 | 2291 |
| SEQ ID NO: 43 | 9 | 7853 |
| SEQ ID NO: 19 | 1 | 2153 |
| SEQ ID NO: 20 | 1 | 2069 |
| SEQ ID NO: 21 | 1 | 2165 |
| SEQ ID NO: 22 | 1 | 2344 |
| SEQ ID NO: 23 | 1 | 2763 |
| SEQ ID NO: 24 | 1 | 2065 |
| SEQ ID NO: 25 | 1 | 2158 |
| SEQ ID NO: 26 | 1 | 2974 |
| SEQ ID NO: 27 | 1 | 3793 |
| SEQ ID NO: 34 | 8 | 6208 |
| SEQ ID NO: 35 | 8 | 5437 |
| SEQ ID NO: 36 | 8 | 4195 |
| SEQ ID NO: 37 | 8 | 5668 |
| SEQ ID NO: 38 | 8 | 4429 |
| SEQ ID NO: 39 | 8 | 4723 |
| SEQ ID NO: 40 | 8 | 4174 |
| SEQ ID NO: 41 | 8 | 4818 |
| SEQ ID NO: 42 | 8 | 4187 |
| SEQ ID NO: 28 | 7 | 4718 |
| SEQ ID NO: 29 | 6 | 4358 |
| SEQ ID NO: 30 | 2 | 2247 |
| SEQ ID NO: 31 | 7 | 3823 |
| SEQ ID NO: 32 | 7 | 4119 |
| SEQ ID NO: 33 | 7 | 6182 |

In summary, the improved resistance to downregulation provided by mutation of lysines in the CD3-zeta intracellular domain of the anti-BCMA CAR was optimal with mutation of all 9 lysines. Mutation of any one lysine in isolation failed to provide resistance to downregulation of CAR in the presence of BCMA ligand. Mutation of any 8 of 9 lysines provided resistance to downregulation that was similar to that of the fully mutated construct. Intermediate numbers of mutations showed intermediate effects. Overall mutation (substitution) of six, seven, eight, or nine of the lysines provided substantial improvements, in proportion to the number of such mutations (substitutions).

### Example 3

A series of experiments was performed to test how substitution of lysine residues in the CD3-zeta intracellular domain of CAR with different amino acids affects resistance of the CAR to downregulation by target ligand.

Numerous IVT mRNA constructs were prepared that encoded variations of the anti-BCMA CAR protein. These constructs were identical with the exception of the intracellular polypeptide sequence encoding the signaling domains. The constructs encoded CAR that comprised either wild-type CD3-zeta intracellular polypeptide sequence (SEQ ID NO: 18), or CD3-zeta intracellular domain sequences with all lysine residues mutated to one of several different amino acids (SEQ ID NO: 43-56). A total of 14 different amino acid variants were tested (Ala, Val, Ile, Leu, Met, Phe, Tyr, Ser, Thr, Asn, Gln, His, Asp, Glu). These IVT mRNA constructs were otherwise identical with respect to the Cap, 5′ UTR, open reading frame encoding the signal peptide, scFv and transmembrane sequences, 3′ UTR and poly A tail. The purpose was to compare the effect of different amino acid substitutions (mutations) on preservation of CAR expression, maintenance of CAR signaling, and resistance of protein to ligand-mediated downregulation following exposure to CAR ligand (BCMA).

For each of the IVT mRNA constructs, CAR T cells were prepared substantially as described in Example 1. 24 hours after transfection, CAR T cells made from each mRNA construct were exposed to MM1S or no target cells by the method described in Example 1. 24 hours later the cells were evaluated for cell count, viability, maintenance of CAR expression, and cytokine production by the method described in Example 1.

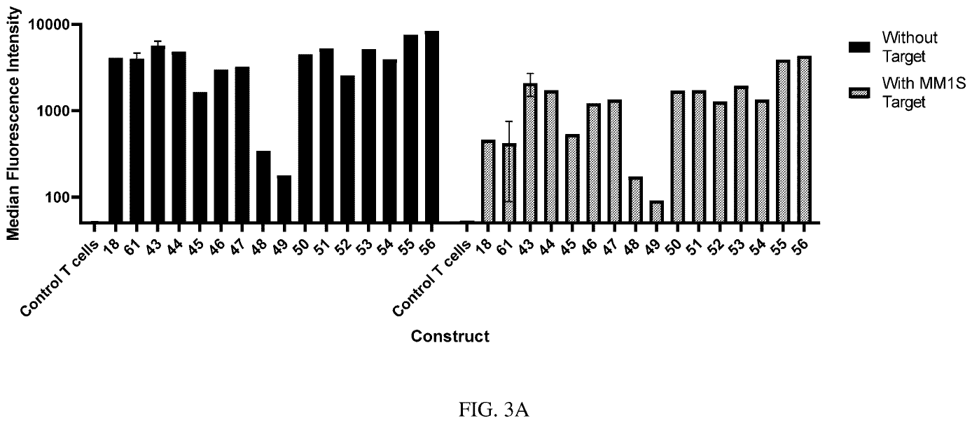

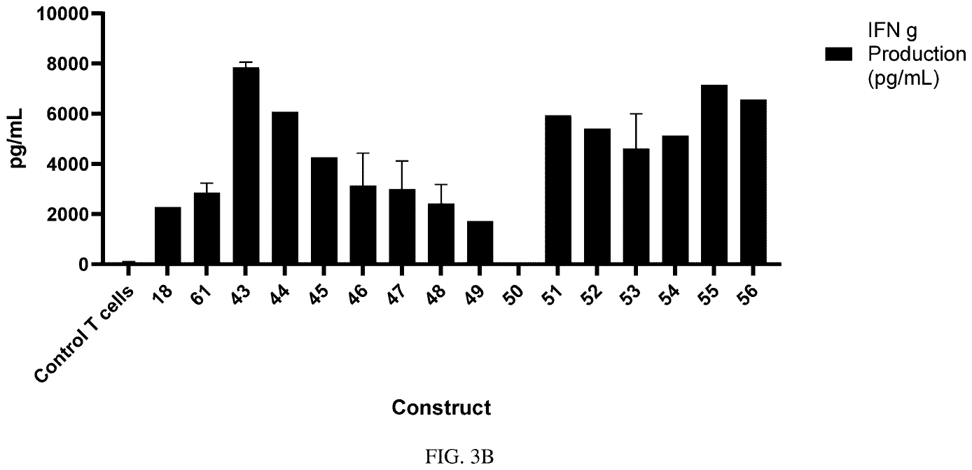

CAR T cells generated with all test constructs showed good viability following electroporation (>70% viability). All constructs tested were capable of driving anti-BCMA CAR expression. In the absence of BCMA ligand, CAR expression by CAR T expressing constructs with lysine-phenylalanine mutations (SEQ ID NO: 48; 340 MFI), lysine-tyrosine mutations (SEQ ID NO: 49; 177 MFI) and lysine-isoleucine mutations (SEQ ID NO: 45; 1,634 MFI) was lower than that of the wild-type construct (SEQ ID NO: 18; 4,084 MFI) (FIG. 3A). Conversely, CAR expression on CAR T cells expressing constructs with lysine-aspartate mutations (SEQ ID NO: 55; 7,562 MFI), lysine-glutamate mutations (SEQ ID NO: 56; 8,346 MFI), and lysine-alanine mutations (SEQ ID NO: 43; 5,656 MFI) was higher than that of the wild-type construct (SEQ ID NO: 18) (FIG. 3A). The other constructs tested showed CAR expression that was comparable to the wild-type constructs (FIG. 3A). All mutated CAR constructs showed an improvement in maintenance of anti-BCMA CAR expression following exposure to ligand (32% to 53%) compared with the wild-type constructs (11%). Among these constructs, the IVT mRNAs comprising SEQ ID NO: 55 (lysine-aspartate intracellular domain mutations) or SEQ ID NO: 56 (lysine-glutamate intracellular domain mutations) showed the highest maintenance of CAR expression following exposure to BCMA ligand (52%) (FIG. 3A). CAR T cells generated with all constructs tested were capable of ligand-dependent signaling, producing interferon-gamma at levels between 1,729 and 7,853 pg/mL during co-culture with MM1S target cells (FIG. 3B). The highest levels of interferon-gamma production were observed using IVT mRNA constructs comprising SEQ ID NO: 55 (lysine-aspartate), SEQ ID NO: 56 (lysine-glutamate), or SEQ ID NO: 43 (lysine-alanine) (FIG. 3B).

TABLE 7
Expression of anti-BCMA CAR by CAR T cells expressing CAR comprising
CD3-zeta intracellular domains with lysines mutated to other amino acids
following culture in the absence or presence of MM1S target cells

| | | | CAR expression | CAR |
|---|---|---|---|---|
| Construct CD3- | Amino Acid in | CAR expression | after exposure to | expression |
| zeta intracellular | place of wild- | without target | BCMA + MM1S | after target |
| domain | type lysine | (MFI) | cells (MFI) | exposure (%) |
| SEQ ID NO: 18 | Lysine (wild- | 4084 | 460 | 11% |
| | type) | | | |
| SEQ ID NO: 43 | Alanine | 5656 | 2079 | 37% |
| SEQ ID NO: 44 | Valine | 4809 | 1725 | 36% |
| SEQ ID NO: 45 | Isoleucine | 1634 | 531 | 32% |
| SEQ ID NO: 46 | Leucine | 2995 | 1212 | 40% |
| SEQ ID NO: 47 | Methionine | 3223 | 1339 | 42% |
| SEQ ID NO: 48 | Phenylalanine | 340 | 172 | 51% |
| SEQ ID NO: 49 | Tyrosine | 177 | 91 | 51% |
| SEQ ID NO: 50 | Serine | 4509 | 1705 | 38% |
| SEQ ID NO: 51 | Threonine | 5245 | 1723 | 33% |
| SEQ ID NO: 52 | Asparagine | 2558 | 1274 | 50% |
| SEQ ID NO: 53 | Glutamine | 5137 | 1936 | 38% |
| SEQ ID NO: 54 | Histidine | 3921 | 1341 | 34% |
| SEQ ID NO: 55 | Aspartate | 7562 | 3903 | 52% |
| SEQ ID NO: 56 | Glutamate | 8346 | 4304 | 52% |

TABLE 8
Cytokine (interferon-gamma) production by anti-BCMA CAR
T cells expressing CAR comprising CD3-zeta intracellular
domain lysine residues mutated to other amino acids following
culture in the presence of MM1S target cells

| Construct CD3-zeta | Amino Acid in place of | Interferon-gamma |
|---|---|---|
| intracellular domain | wild-type Lysine | (pg/mL) |
| SEQ ID NO: 18 | Lysine (wild-type) | 2291 |
| SEQ ID NO: 43 | Alanine | 7853 |
| SEQ ID NO: 44 | Valine | 6085 |
| SEQ ID NO: 45 | Isoleucine | 4266 |
| SEQ ID NO: 46 | Leucine | 3138 |
| SEQ ID NO: 47 | Methionine | 2993 |
| SEQ ID NO: 48 | Phenylalanine | 2427 |
| SEQ ID NO: 49 | Tyrosine | 1729 |
| SEQ ID NO: 50 | Serine | n.d. |
| SEQ ID NO: 51 | Threonine | 5950 |
| SEQ ID NO: 52 | Asparagine | 5412 |
| SEQ ID NO: 53 | Glutamine | 4617 |
| SEQ ID NO: 54 | Histidine | 5125 |
| SEQ ID NO: 55 | Aspartate | 7158 |
| SEQ ID NO: 56 | Glutamate | 6568 |

n.d.—not determined

In summary, the improved resistance to downregulation provided by mutation of lysines in the CD3-zeta intracellular domain of the anti-BCMA CAR was compatible with their mutation to a variety of different amino acids including alanine, valine, isoleucine, leucine, methionine, serine, threonine, asparagine, glutamine, histidine, aspartate, and glutamate. CAR constructs in which the lysine amino acids were mutated to aspartate, glutamate, or alanine showed the highest preservation of CAR expression upon exposure to BCMA⁺ target cells and highest level of signaling.

### Example 4

A series of experiments were conducted to test how inclusion of additional costimulatory domains in the intracellular part of lysine-alanine mutated CAR receptor affects resistance of the CAR to downregulation by target ligand BCMA.

A series of IVT mRNA constructs were prepared that encoded variations of the anti-BCMA CAR protein. These constructs were identical with the exception of the intracellular polypeptide sequence encoding the intracellular domains. The constructs encoded a protein that comprised either wild-type intracellular CD3-zeta polypeptide sequence (SEQ ID NO: 18), or intracellular CD3-zeta polypeptide sequence with all lysine residues mutated to alanine (SEQ ID NO: 43), or an intracellular CD3-zeta polypeptide sequence with all lysine residues mutated to alanine in addition to a preceding costimulatory sequence, namely: a wild-type CD28 costimulatory domain (SEQ ID NO: 57), a CD28 costimulatory domain with lysines mutated to alanine (SEQ ID NO: 58), a wild-type 41BB costimulatory domain (SEQ ID NO: 59), or a 41BB costimulatory domain with all lysines mutated to alanine (SEQ ID NO: 60). The constructs were otherwise identical with respect to the Cap, 5′ UTR, open reading frame encoding the signal peptide, scFv and transmembrane sequences, 3′ UTR and poly A tail. The experiment then tested the compatibility of wild-type or lysine-alanine mutated CD28 and 41BB costimulatory domains to preserve CAR expression, maintain CAR signaling, and provide resistance of the CAR protein to ligand-mediated downregulation following exposure to CAR ligand (BCMA).

For each of the IVT mRNA constructs, CAR T cells were prepared substantially as described in Example 1. 24 hours after transfection, CAR T cells made from each mRNA construct were exposed to MM1S or no target cells by the method described in Example 1. 24 hours later the cells were evaluated for cell count, viability maintenance of CAR expression, cytotoxicity, and cytokine production by the method described in Example 1. The constructs and their effect on downregulation of CAR expression are shown below.

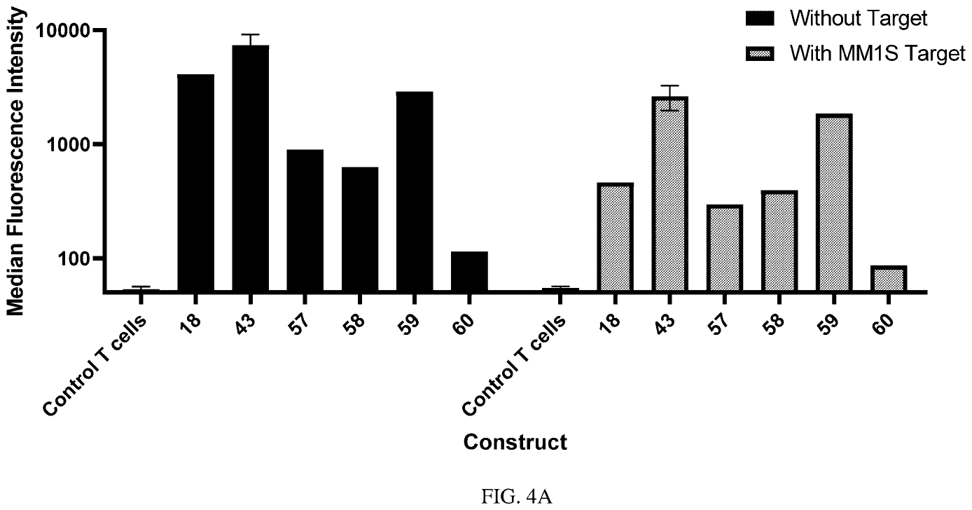

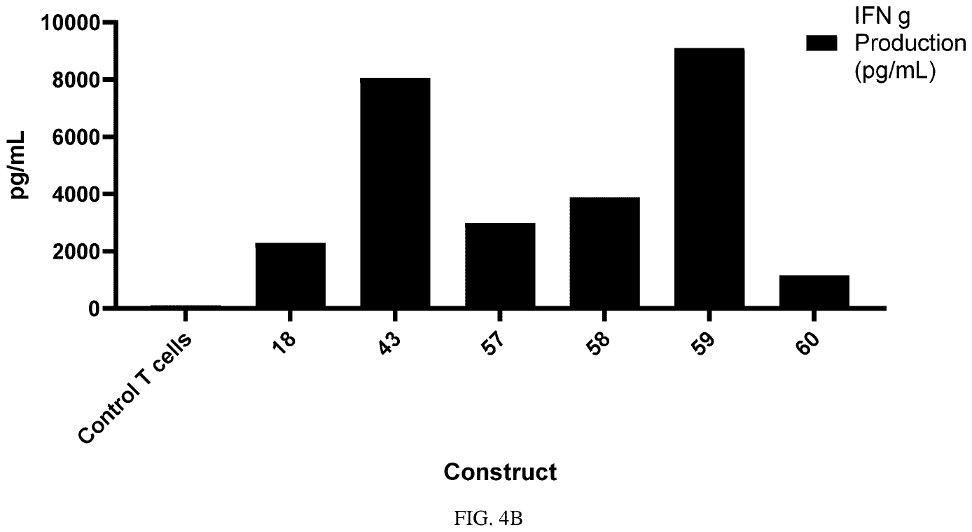

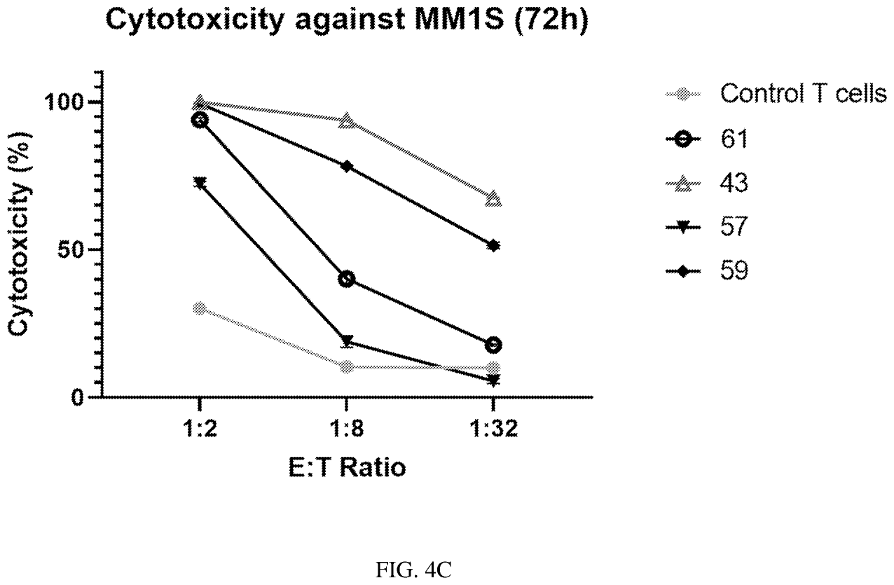

CAR T cells generated with all test constructs showed high viability following electroporation. CAR expression in CAR T cells generated using constructs encoding a protein that only contained the intracellular CD3-zeta signaling domain, SEQ ID NO: 18 (wild-type) and SEQ ID NO: 43 (K-A), showed highest anti-BCMA CAR expression in the absence of BCMA ligand (FIG. 4A). Addition of a wild-type CD28 (SEQ ID NO: 57) or 41BB (SEQ ID NO: 59) signaling domain led to reduced CAR expression in the absence of ligand (896 MFI and 2,886 MFI compared with 7,389 MFI for SEQ ID NO: 43) (FIG. 4A). Mutation of the costimulatory domains by mutation of lysine to alanine (SEQ ID NOs: 58 and 60) resulted in further loss of anti-BCMA CAR expression in absence of ligand (625 MFI and 114 MFI, respectively) (FIG. 4A). In the presence of ligand, CAR T cells generated with all constructs encoding a protein that contained a mutated lysine-alanine CD3-zeta signaling domain showed higher maintenance of CAR expression on the cell surface following exposure to BCMA+ MM1S target cells (35%-75%) compared with that on the construct SEQ ID NO: 18 comprising wild-type CD3-zeta (11%) (FIG. 4A). Similarly, CAR T cells generated with all constructs could signal and produce interferon-gamma upon exposure to MM1S target cells (FIG. 4B). Interferon-gamma production was highest for construct SEQ ID NO: 59 comprising a wild-type 41BB signaling domain and a K-A mutant CD3-zeta signaling domain (9,101 pg/mL), followed by SEQ ID NO: 43 comprising a K-A mutant CD3-zeta signaling domain alone (8,054 pg/mL) (FIG. 4B). Interferon-gamma production driven by constructs SEQ ID NO: 57 and 58 that comprised wild-type and K-A mutant CD28 signaling domains, respectively, (2,998 and 3,886 pg/mL) was higher than the wild-type CAR construct SEQ ID NO: 18 (2,291 pg/mL) (FIG. 4B). Cytotoxicity of CAR T cells generated with separate constructs comprising CD3-zeta only, CD28-CD3-zeta, and 41BB-CD3-zeta signaling domains was evaluated by pre-exposure of cells to MM1S followed by a cytotoxicity assay against BCMA+ MM1S-GFP cells for 72 hours. All constructs tested (SEQ ID NOs: 61, 43, 57, and 59) displayed a high degree of cytotoxicity against the MM1S

GFP target cell at a 1:2 effector:target ratio compared with control T cells (FIG. 4C). At lower effector:target ratios, CAR T cells generated with SEQ ID NO: 43 (comprising a K-A CD3-zeta signaling domain), and SEQ ID NO: 59 (comprising a WT 41BB and a K-A CD3-zeta signaling domain) showed high levels of specific cytotoxicity against MM1S-GFP (67.4% and 51.4%, respectively, at the 1:32 effector:target ratio) that were 3- to 4-fold higher than those observed with the wild-type construct (SEQ ID NO: 61, comprising a wild-type CD3-zeta signaling domain alone) (FIG. 4C).

TABLE 9
Expression of anti-BCMA CAR by CAR T cells expressing CAR comprising
CD3-zeta intracellular domains with lysines mutated to alanine and wild-
type or mutant co-stimulation domains in the intracellular domain

| | | CAR | CAR expression | CAR |
|---|---|---|---|---|
| Construct | | expression | after exposure to | expression |
| intracellular | Signaling | without target | BCMA + MM1S | after target |
| domain | Domains | (MFI) | cells (MFI) | exposure (%) |
| SEQ ID NO: 18 | CD3-zeta WT | 4084 | 460 | 11% |
| SEQ ID NO: 43 | CD3-zeta K-A | 7389 | 2617 | 35% |
| SEQ ID NO: 57 | CD28WT, CD3- | 896 | 295 | 33% |
| | zeta K-A | | | |
| SEQ ID NO: 58 | CD28 K-A, | 625 | 394 | 63% |
| | CD3-zeta K-A | | | |
| SEQ ID NO: 59 | 41BB WT, CD3- | 2886 | 1845 | 64% |
| | zeta K-A | | | |
| SEQ ID NO: 60 | 41BB K-A, | 114 | 86 | 75% |
| | CD3-zeta K-A | | | |

TABLE 10
Cytokine (interferon-gamma) production by anti-BCMA CAR
T cells expressing CAR comprising CD3-zeta intracellular
domain lysines mutated to alanine and wild-type or mutant
co-stimulation domains in the intracellular domain.

| Construct | | Interferon-gamma |
|---|---|---|
| intracellular domain | Signaling Domains | (pg/mL) |
| SEQ ID NO: 18 | CD3-zeta WT | 2291 |
| SEQ ID NO: 43 | CD3-zeta K-A | 8054 |
| SEQ ID NO: 57 | CD28WT, CD3-zeta K-A | 2998 |
| SEQ ID NO: 58 | CD28 K-A, CD3-zeta K-A | 3886 |
| SEQ ID NO: 59 | 41BB WT, CD3-zeta K-A | 9101 |
| SEQ ID NO: 60 | 41BB K-A, CD3-zeta K-A | 1165 |

TABLE 11
CAR T cell 72-hour cytotoxicity against MM1S-GFP
cells following exposure to BCMA ligand (MM1S)

| Construct | | % MM1S-GFP | % MM1S-GFP | % MM1S-GFP |
|---|---|---|---|---|
| intracellular | Signaling | Cytotoxicity | Cytotoxicity | Cytotoxicity |
| domain | Domains | (1:2) | (1:8) | (1:32) |
| Control T cells | N.A. | 30.1 ± 3.6 | 10.3 ± 2.3 | 9.9 ± 1 |
| SEQ ID NO: 61 | CD3-zeta WT | 93.9 ± 1.3 | 40.1 ± 3.6 | 17.7 ± 0.6 |
| SEQ ID NO: 43 | CD3-zeta K-A | 99.8 ± 0.1 | 93.8 ± 0.2 | 67.4 ± 0.2 |
| SEQ ID NO: 57 | CD28WT, CD3- | 72.2 ± 1.5 | 18.8 ± 3.4 | 5.6 ± 1.4 |
| | zeta K-A | | | |
| SEQ ID NO: 59 | 41BB WT, CD3- | 99.4 ± 0.1 | 78.2 ± 0.7 | 51.4 ± 1.8 |
| | zeta K-A | | | |

mean ± SD (n = 3)

In summary, CAR constructs encoding proteins comprising CD3-zeta intracellular signaling domains that were rendered resistant to ligand-dependent downregulation by mutation of lysine amino acids were successfully combined with additional signaling domains from CD28 and 41BB. Addition of these domains did not interfere with prevention of downregulation even in the context of wild-type CD28 and 41BB signaling domain sequences. CAR constructs comprising mutated CD28-CD3-zeta, or 41BB-CD3-zeta, intracellular signaling domains were capable of efficiently driving CAR-dependent cytokine production and cytotoxicity.

### Example 5

Experiments were conducted to test how inclusion of a CD28 costimulatory domain in the intracellular part of lysine-alanine mutated or lysine-glutamate mutated CAR receptor affected resistance of the CAR to downregulation by target BCMA.

A series of IVT mRNA constructs were prepared that encoded variations of the anti-BCMA CAR protein. These constructs were identical with the exception of the intracellular polypeptide sequence encoding the signaling domains. A control construct encoded a protein that comprised wild-type intracellular CD3-zeta polypeptide sequence (SEQ ID NO: 18). Two series of test constructs encoded proteins that comprised either intracellular CD3-zeta polypeptide sequence with all lysine residues mutated to alanine (SEQ ID NOs: 43, 57, 58, 62), or intracellular CD3-zeta polypeptide sequence with all lysine residues mutated to glutamate (SEQ ID NOs: 56, 63, 64, 65). The constructs varied in presence and sequence composition of the CD28 costimulatory domain of the encoded protein: two control test constructs that encoded a protein that lacked the CD28 costimulatory domain (SEQ ID NOs: 43, 56); two constructs encoded a protein that comprised wild-type CD28 costimulatory domains (SEQ ID NOs: 57, 63); two constructs encoded a protein that comprised a CD28 costimulatory domain with lysine residues mutated to alanine (SEQ ID NOs: 58; 64); and two constructs encoded a protein that comprised a CD28 costimulatory domain with lysines mutated to glutamate (SEQ ID NOs: 62, 65). The constructs were otherwise identical with respect to the Cap, 5′ UTR, open reading frame encoding the signal peptide, scFv and transmembrane sequences, 3′ UTR, and poly A tail.

The experiment assessed the capacity of second-generation CAR comprising lysine-alanine or lysine-glutamate mutated CD28-CD3-zeta intracellular polypeptides to preserve CAR expression, maintain CAR signaling, and provide resistance of the CAR protein to ligand-mediated downregulation following exposure to CAR ligand (BCMA).

For each of the IVT mRNA constructs, CAR T cells were prepared substantially as described in Example 1. 24 hours after transfection, CAR T cells made from each mRNA construct were exposed to MM1S or no target cells by the method described in Example 1. 24 hours later the cells were evaluated for cell count, viability maintenance of CAR expression, cytotoxicity, and cytokine production by the method described in Example 1. The constructs and their effect on downregulation of CAR expression are shown below.

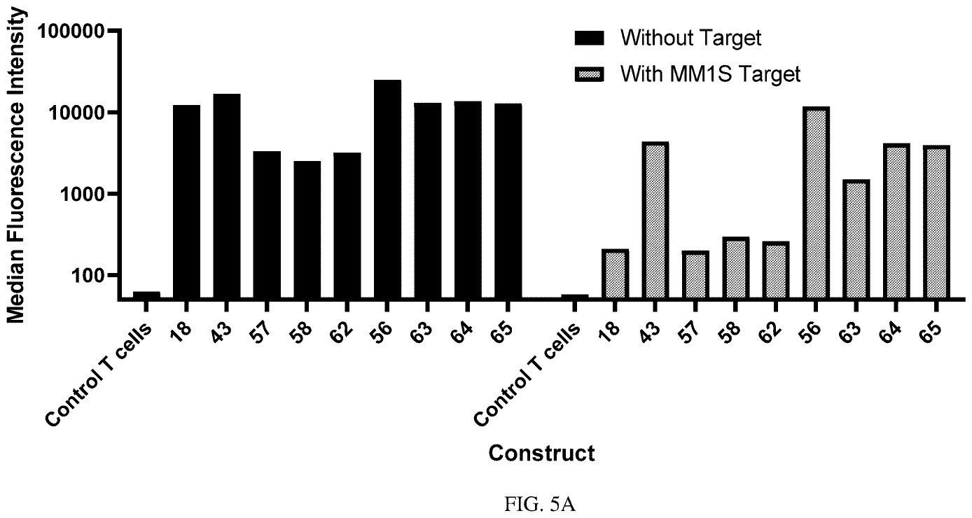

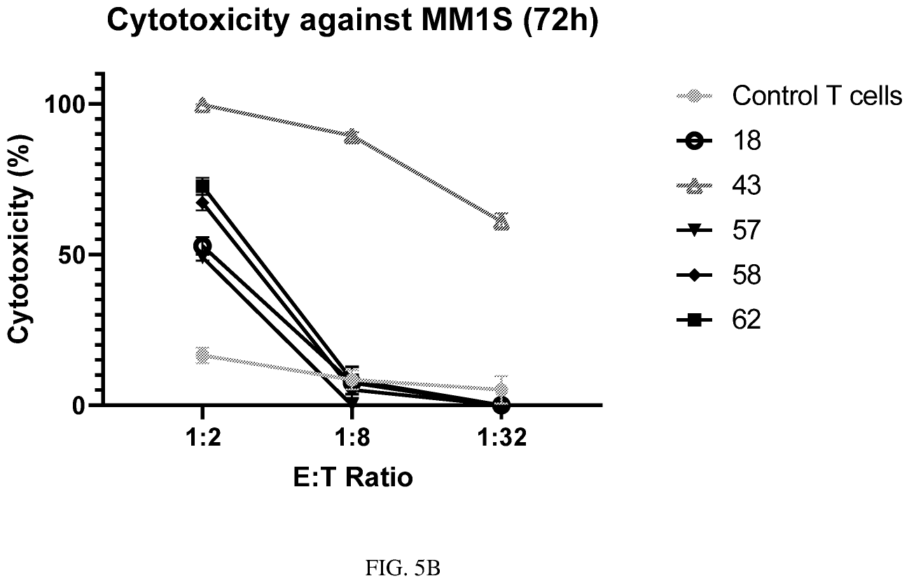

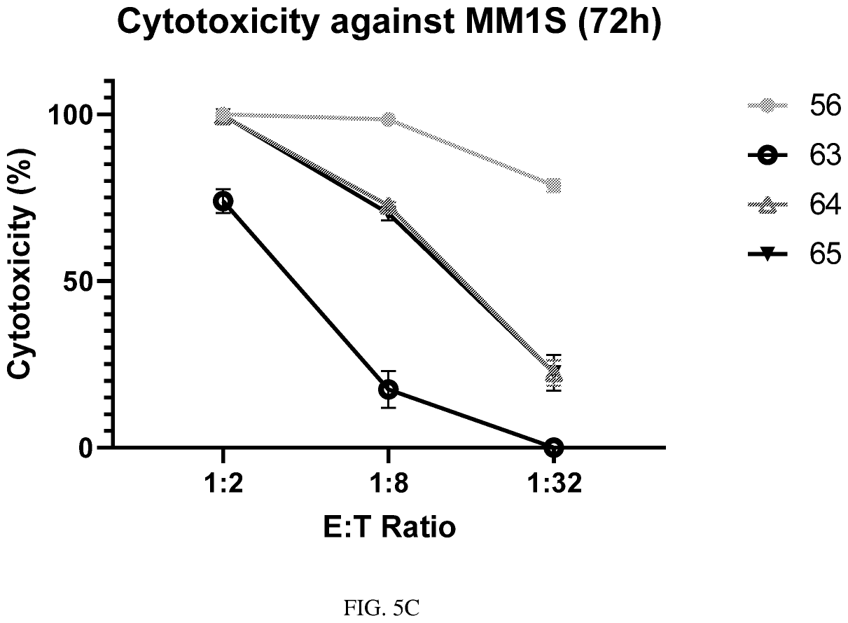

CAR T cells generated with all test constructs showed high viability following electroporation. CAR expression CAR T cells generated using constructs that encoded a protein that comprised the intracellular CD3-zeta signaling domain, SEQ ID NO: 18 (wild-type), SEQ ID NO: 43 (K-A), and SEQ ID NO: 56 (K-E) showed high anti-BCMA CAR expression in the absence of BCMA ligand. Addition of the CD28 costimulatory domain resulted in a reduction in CAR expression compared with the CD3-zeta-alone constructs (2,507-3,306 MFI for SEQ ID NOs: 57, 58, 62 compared with 16,848 MFI for SEQ ID NO: 43 in the context of lysine-alanine mutations, and 12,784-13,598 MFI for SEQ ID NOs: 63, 64, 65 compared with 24,850 MFI for SEQ ID NO: 56 in the context of lysine-glutamate mutations) (FIG. 5A). In the presence of BCMA, CAR T cells generated with constructs that encoded a protein that contained a mutated lysine-alanine CD3-zeta signaling domain (SEQ ID NO: 43, 57, 58, 62) or lysine-glutamate CD3-zeta signaling domain (SEQ ID NO: 56, 63, 64, 65) showed higher maintenance of CAR expression on the cell surface following exposure to BCMA+ MM1S target cells (6-26% and 12-47%, respectively) compared with that on the construct SEQ ID NO: 18 comprising wild-type CD3-zeta (2%) (FIG. 5A). The maintenance of CAR expression following exposure to BCMA ligand was highest in CAR T cells generated with constructs encoding proteins comprising lysine-glutamate mutations in the CD3-zeta intracellular domain, including those with no costimulatory domain (SEQ ID NO: 56), or a CD28 costimulatory domain in which lysine residues were mutated to alanine (SEQ ID NO: 63) or a costimulatory domain in which lysine residues were mutated to glutamate (SEQ ID NO: 64), that showed 47%, 30% and 31% maintenance of surface CAR expression respectively (FIG. 5A). Cytotoxicity of CAR T cells generated with constructs comprising CD3-zeta only and CD28-CD3-zeta signaling domains was evaluated by pre-exposure of cells to MM1S followed by a cytotoxicity assay against BCMA+ MM1S-GFP cells for 72 hours. All constructs tested (SEQ ID NOs: 43, 56, 57, 58, 62, 63, 64, and 65) conferred a high degree of cytotoxicity against the MM1S-GFP target cell at a 1:2 effector:target ratio compared with control T cells. At lower effector:target ratios, CAR T cells generated with SEQ ID NO: 43 (comprising a K-A CD3-zeta signaling domain alone), SEQ ID NO: 56 (comprising a K-E CD3-zeta signaling domain alone), SEQ ID NO: 63 (comprising a K-A CD28 signaling domain and a K-E CD3-zeta signaling domain) and SEQ ID NO: 64 (comprising a K-E CD28 signaling domain and a K-E CD3-zeta signaling domain) conferred high levels of specific cytotoxicity against MM1S-GFP (89.4%, 98.4%, 72.7%, and 70.2%, respectively, at the 1:8 effector:target ratio) that were 9- to 13-fold higher than those observed (7.5%) with the wild-type construct (SEQ ID NO: 18, comprising a wild-type CD3-zeta signaling domain alone) (FIGS. 5B and 5C).

TABLE 12
Expression of anti-BCMA CAR by CAR T cells expressing CAR comprising CD3-
zeta intracellular domains with lysines mutated to alanine or glutamate,
and wild-type or mutant co-stimulation domains in the intracellular domain

| | | CAR | CAR expression | |
|---|---|---|---|---|
| Construct | | expression | after exposure to | CAR expression |
| intracellular | Signaling | without target | BCMA + MM1S | after target |
| domain | Domains | (MFI) | cells (MFI) | exposure (%) |
| SEQ ID NO: 18 | CD3-zeta WT | 12151 | 210 | 2% |
| SEQ ID NO: 43 | CD3-zeta K-A | 16848 | 4371 | 26% |
| SEQ ID NO: 57 | CD28 WT, | 3306 | 199 | 6% |
| | CD3-zeta K-A | | | |
| SEQ ID NO: 58 | CD28 K-A, | 2507 | 296 | 12% |
| | CD3-zeta K-A | | | |
| SEQ ID NO: 62 | CD28 K-E, | 3174 | 261 | 8% |
| | CD3-zeta K-A | | | |
| SEQ ID NO: 56 | CD3-zeta K-E | 24850 | 11754 | 47% |
| SEQ ID NO: 63 | CD28 WT, | 12974 | 1498 | 12% |
| | CD3-zeta K-E | | | |
| SEQ ID NO: 64 | CD28 K-A, | 13598 | 4139 | 30% |
| | CD3-zeta K-E | | | |
| SEQ ID NO: 65 | CD28 K-E, | 12784 | 3932 | 31% |
| | CD3-zeta K-E | | | |

TABLE 13
CAR T cell 72-hour cytotoxicity against MM1S-GFP
cells following exposure to BCMA ligand (MM1S)

| Construct | | % MM1S-GFP | % MM1S-GFP | % MM1S-GFP |
|---|---|---|---|---|
| intracellular | Signaling | Cytotoxicity | Cytotoxicity | Cytotoxicity |
| domain | Domains | (1:2) | (1:8) | (1:32) |
| Control T cells | N.A. | 16.6 ± 4.5 | 8.5 ± 5.6 | 5.2 ± 7.9 |
| SEQ ID NO: 18 | CD3-zeta WT | 52.8 ± 5.1 | 7.5 ± 8.8 | −3 ± 2.1 |
| SEQ ID NO: 43 | CD3-zeta K-A | 99.7 ± 0 | 89.4 ± 2.1 | 61 ± 4.6 |
| SEQ ID NO: 57 | CD28 WT, CD3- | 49.1 ± 2 | −7.3 ± 2.4 | −17.6 ± 5.5 |
| | zeta K-A | | | |
| SEQ ID NO: 58 | CD28 K-A, CD3- | 67.3 ± 4.5 | 5.2 ± 2.7 | −14.7 ± 2.2 |
| | zeta K-A | | | |
| SEQ ID NO: 62 | CD28 K-E, CD3- | 72.7 ± 4.8 | 8.4 ± 8 | −3.6 ± 5.8 |
| | zeta K-A | | | |
| SEQ ID NO: 56 | CD3-zeta K-E | 100 ± 0 | 98.4 ± 0.6 | 78.6 ± 3.4 |
| SEQ ID NO: 63 | CD28 WT, CD3- | 73.9 ± 6.2 | 17.5 ± 9.5 | −9.1 ± 7.1 |
| | zeta K-E | | | |
| SEQ ID NO: 64 | CD28 K-A, CD3- | 99.5 ± 0.3 | 72.7 ± 1.8 | 22.4 ± 6.6 |
| | zeta K-E | | | |
| SEQ ID NO: 65 | CD28 K-E, CD3- | 99.6 ± 0.1 | 70.2 ± 3.6 | 22.5 ± 9.2 |
| | zeta K-E | | | |

mean ± SD (n = 3)

In summary, CAR constructs encoding proteins comprising CD3-zeta intracellular signaling domains rendered resistant to ligand-dependent downregulation by mutation of lysine amino acids were combined with a CD28 signaling domain. Addition of a CD28 signaling domain to a lysine-glutamate mutated CD3-zeta intracellular signaling domain was compatible with prevention of CAR downregulation by BCMA ligand. This effect was most prominent when the CD28 lysines were mutated to alanine or glutamate. In contrast, addition of a CD28 signaling domain to a lysine-alanine mutated CD3-zeta intracellular signaling domain showed dramatically lower prevention of CAR downregulation by BCMA ligand. Thus, mutation of lysines, e.g., to glutamate, throughout the CD28-CD3-zeta intracellular sequence results in a CAR that is highly resistant to BCMA ligand-dependent downregulation.

### Example 6

A series of experiments was conducted to test how downregulation-resistant anti-BCMA CAR comprising lysine-alanine mutations in the intracellular signaling domain activates CAR T cells and drives effector functions in response to normal plasmablasts and plasmacytoid Dendritic Cells (pDCs).

In autoimmune diseases such as SLE, plasmablasts and further differentiated plasma cells produce autoantibody contributing to pathogenesis, whereas pDCs are activated by inflammatory triggers such as immune complexes and drive a pathogenic immune response through production of cytokines and stimulation of T cells. The capacity of downmodulation resistant anti-BCMA CAR to drive CAR-specific T cell activation and modulate the immune response in the context of autologous plasmablasts and pDCs was determined.

An IVT mRNA construct was prepared encoding an anti-BCMA CAR protein with an intracellular CD3-zeta polypeptide sequence with all Lysine residues mutated to Alanine (SEQ ID NO: 43). For this construct, CAR T cells were prepared substantially as described in Example 1. Plasmablasts and pDCs were isolated from whole blood of the same (autologous) donors by antibody-magnetic bead-based isolation (plasma cell isolation kit II and diamond plasmacytoid dendritic cell isolation Kit II, human, Miltenyi). The isolated plasmablasts and pDCs showed staining with characteristic cell surface markers CD38/CD138 and CD123, respectively. Co-cultures were established using BCMA CAR T (SEQ ID NO: 43) or control CD8+ T cells without CAR and each of the autologous plasmablast and pDC populations (for plasmablasts, 7.5K T cells and 1.5K plasmablasts; and for pDCs, 10K T cells and 10K pDC). Cultures were incubated at 37° C. overnight and were analyzed the following day for activation of the CAR T cells or control cells using activation induced markers CD69 and CD137 (41BB) by flow cytometry, and for interferon-gamma production in the supernatant by ELISA.

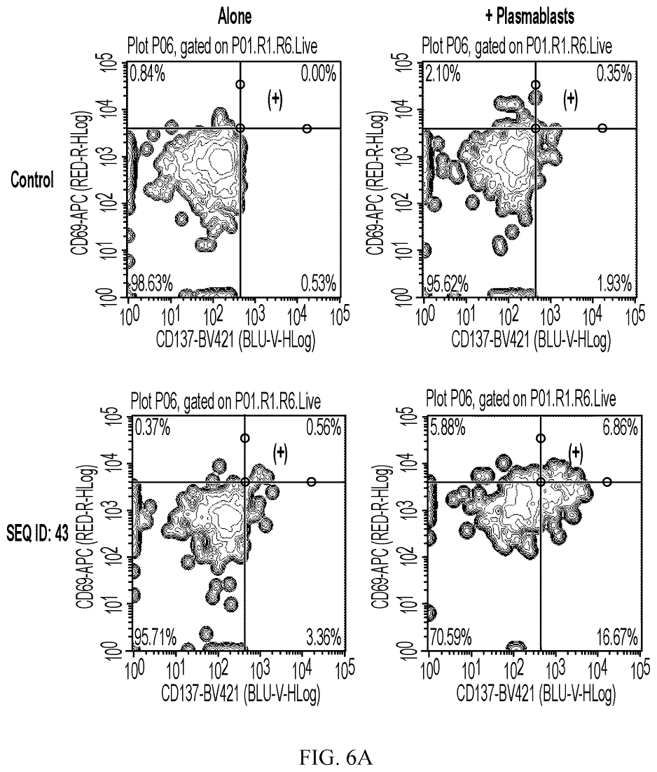

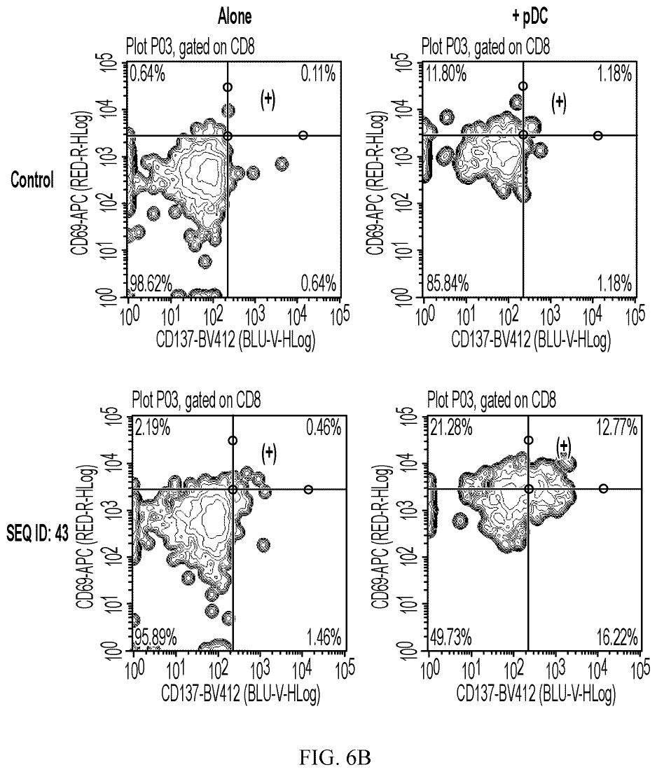

The BCMA CAR T cells generated with an IVT mRNA encoding a protein comprising the sequence of SEQ ID NO: 43 showed upregulation of CD69 and 41BB in response to exposure to plasmablasts and pDCs (for plasmablast co-culture CD69: from 0.93 to 12.74%, 41BB: from 3.92 to 23.93%; for pDC co-culture, CD69: from 2.65 to 34.05%, 41BB: from 1.92 to 28.99%) (FIGS. 6A and 6B). Similarly, interferon-gamma was produced in the supernatant of the co-cultures (187 pg/mL in the plasmablast co-culture and 5 pg/mL in the pDCs co-culture). In contrast, control cells that lacked the IVT mRNA showed no upregulation of activation markers in response to plasmablasts and pDCs (for plasmablast co-culture, 2.45% CD69+ and 2.28% 41BB+, and for pDC co-culture 12.98% CD69+ and 2. 36% 41BB+), and interferon-gamma remained at concentrations 5-25-fold lower than with the BCMA CAR T (7.3 pg/mL in the plasmablast co-culture and <LOD in the pDC co-culture) (FIGS. 6A and 6B). Thus, CAR T cells expressing a downmodulation-resistant CAR activate in a CAR-dependent manner in the context of plasmablasts and pDCs and produce immunomodulatory cytokines of a type known to regulate autoimmune disease.

### Example 7

This example describes an experiment and process to confer resistance to downregulation to each of a plurality of CAR proteins, each expressed in CAR T cells (thus each separately targeting CD19, PSMA, and CCR4) by mutation of intracellular lysine amino acid residues to alanine or glutamate.

A series of mRNA constructs is prepared that encode variations of CAR proteins of interest that each comprise an scFv specific to one of the following cell surface targets: BCMA, CD19, PSMA, or CCR4. For each such cell surface target, mRNA constructs are made that encode a CAR comprising either a wild-type intracellular CD3-zeta polypeptide sequences (SEQ ID NO: 18), a wild-type intracellular CD28-CD3-zeta polypeptide sequence (SEQ ID NO: 66), an intracellular CD3-zeta polypeptide sequence with all lysine residues mutated to alanine (SEQ ID NO: 43), an intracellular CD3-zeta polypeptide sequence with all lysine residues mutated to glutamate (SEQ ID NO: 56), or an intracellular CD28-CD3-zeta polypeptide sequence with all lysine residues mutated to glutamate (SEQ ID NO: 65). These constructs are otherwise identical with respect to the Cap, 5′ UTR′, 3′ UTR and poly A tail.

For each of the mRNA constructs, CAR T cells are prepared substantially as described in Example 1. About 24 hours after transfection, CAR T cells made from each mRNA construct are exposed to different target cell lines (for BCMA CAR, MM1S; for CD19 CAR, Raji; for PSMA CAR, LNCaP; and for CCR4 CAR, CEM), or no target cells, according to the method described in Example 1. 24 hours later the cells are evaluated for cell count, viability, expression of CAR (using BCMA-APC for BCMA CAR, FMC63 antibody for CD19 CAR, PSMA-PE for PSMA CAR, and anti-idiotype antibody for CCR4), by the method described in Example 1. The pre-exposed CAR T cells are then evaluated for their ability to perform effector function (cytotoxicity and interferon-gamma production) in response to additional target cell lines by the method substantially described in Example 1.

It is expected that CAR T cells generated using IVT mRNA with specificity for BCMA, CD19, PSMA, and CCR4 that comprise lysine-alanine or lysine-glutamate mutations in the CD3-zeta or CD28-CD3-zeta intracellular signaling domains will show less downregulation and higher cytotoxicity and cytokine production than the wild-type counterparts.

### Example 8

This example describes an experiment and process to control tumor burden in mice using CAR T cells generated with IVT mRNA encoding BCMA CAR that is resistant to ligand induced downregulation. Different mRNA CAR constructs separately comprise the sequences of SEQ ID NOs: 17, 67, 98, or 99. These mRNA constructs encode CAR proteins that comprise, respectively, the sequences of SEQ ID NOs: 5, 68, 101, or 102.

CAR T cells are prepared by transfection of the mRNA CAR construct into human CD8+ cells, as described in Example 1, with the exception that the endogenous T cell receptors are genetically removed from the cells by CRISPR/Cas engineering. Negative controls used in this example include control CD8+ cells without the IVT mRNA. NOD-scid-gamma (NSG) mice are inoculated with 2 million MM1S-fluc human multiple myeloma tumor cells. Tumor burden is monitored by serial bioluminescence imaging. On Day 5 mice are randomized, then receive by intravenous injection the control CD8+ T Cells, or CAR T cells transfected to express a CAR of SEQ ID NOs: 5 or 68. Tumor burden is measured daily to confirm reduced burden over time in mice treated with vehicle or control CD8+ cells compared against CAR T cells transfected to express a CAR of SEQ ID NOs: 5 or 68. It is expected that CAR T cells generated with (SEQ ID NOs: 5 or 68) will significantly control tumor burden and inhibit tumor growth.

In conclusion, CAR T Cells prepared from three examples of the anti-BCMA CAR-encoding mRNA constructs of the invention (and their corresponding CAR proteins) are expected to inhibit growth of human myeloma tumors in a predictive animal model.

### Example 9

This example describes an experiment and process to cause T cells to express one or more downregulation-resistant CAR proteins using carriers and nucleic acids other than IVT mRNA.

CAR T cells are prepared and modified by use of the Sleeping Beauty transposon system to express CAR proteins of the present disclosure. A “Wild-Type CAR” plasmid is constructed comprising the elements of an EF1a promoter, an IgG 5′ UTR, an open reading frame encoding the amino acid sequence of SEQ ID NO: 1 and a poly adenylation sequence, wherein the foregoing elements were collectively flanked by the Inverted Terminal Repeats of the Sleeping Beauty transposon. A “K-E CAR” plasmid (wherein K-E refers to amino acid substitutions) is constructed comprising the elements of an EF1a promoter, an IgG 5′ UTR, an open reading frame encoding the amino acid sequence of SEQ ID NO: 5 and a poly adenylation sequence, wherein the foregoing elements were collectively flanked by the Inverted Terminal Repeats of the Sleeping Beauty transposon. An “SB11” Transposase plasmid is constructed comprising an EFI a promoter, an IgG 5′ UTR, a Kozak consensus sequence, an open reading frame encoding SB II, and a polyadenylation sequence. To generate wild-type CAR T cells, peripheral blood mononuclear cells from a normal human donor are washed and resuspended in P3 buffer (Lonza) in the presence of both the Wild-Type CAR Transposon plasmid and the SB11 plasmid. The same strategy is used to generate lysine-mutated CAR T cells, using the K-F. CAR plasmid and the SB11 plasmid. Cells are electroporated (4D Nucleofector, Lonza) to introduce the plasmid(s) into cells. The cells are then transferred to culture and stimulated with CD3/CD28 Dynabeads® (ThermoFisher). During expansion CD4+ and CD8+ T cells are analyzed for expression of the CAR by staining with BCMA-APC reagent and flow cytometry. It is expected that T cells will expand and express the anti-BCMA CAR proteins for at least 14 days.

CAR expression and functional CAR T cell activity are evaluated using cytotoxicity assays with the multiple myeloma target cell line MM1S-GFP as described in Example 1. It is expected that CAR T cells generated with transposon encoding a lysine-glutamate mutated intracellular signaling domain (SEQ ID NO: 5) show higher expression levels of BCMA CAR and higher potency in cytotoxicity assays than the CAR T cells generated with transposons encoding the wild-type CAR.

### Example 10

This example describes a strategy to eradicate myeloma cells and treat disease in patients with multiple myeloma (MM) using anti-BCMA CAR T cells expressing one or more CAR proteins of the present disclosure.

Anti-BCMA CAR T cells are prepared substantially according to the methods of Example 1 using the CAR proteins of the present disclosure comprising a sequence of SEQ ID NOs: 3, 4, 5, 68, 101, or 102. Patients with MM (optionally prepared with lymphodepleting chemotherapy) are infused with 0.2 to 100×10⁹ anti-BCMA CAR T cells of the present disclosure. Serum M-protein levels, free light chains of the MM-related immunoglobulin, soluble serum BCMA levels, peripheral blood CAR+ T cell counts, serum cytokine levels (e.g., interferon-gamma, IL-2, IL-10), and bone marrow biopsies are analyzed before and at 2, 4, 8, 12 and 24 weeks after treatment. It is expected that CAR T cells generated with the CAR described in the present disclosure effectively will reduce and/or eradicate the MM, as measured hv reduction of serum M-protein levels, free light chains of the MM-related immunoglobulin, soluble serum BCMA levels, and MM cells in bone marrow biopsies.

### Example 11

This example describes an experiment and process to control autoimmune disease using anti-BCMA CAR T cells expressing one or more CAR proteins of the present disclosure.

Anti-BCMA CAR T cells are prepared substantially according to the methods of Example 1 using CAR receptors comprising a sequence of SEQ ID NOs: 3, 4, 5, 68, 101, or 102. Patients with MG (optionally prepared with lymphodepleting chemotherapy) are infused with 0.2 to 100×10⁹ anti-BCMA CAR T cells of the present disclosure. Anti-autoantigen antibodies (e.g., anti-AChR or anti-MUSK), soluble serum BCMA levels, peripheral blood CAR+ T cell counts, serum cytokine levels (e.g., TNF, IL-6, IL-2, interferon-gamma, IL-10), and clinical assessment of disease such as the Myasthenia Gravis Activities of Daily Living Scale (MG-ADL) are assessed in patients at 2, 4, 8, 12, 24, and 52 weeks after treatment. It is expected that, CAR T cells generated with a CAR of the present disclosure will effectively control the autoimmune disease as measured by reduction of auto-antibody levels, reduction in circulating cytokine concentrations and reduced clinical manifestations of disease as measured by decreases in the MG-ADL or other clinical score.

### Example 12

This example describes an experiment to test how substitution of intracellular Lysine residues in the CD3-zeta and CD28 intracellular domain of an anti-CD19 CAR to Glutamic Acid affects resistance to downregulation by target ligand CD19.

A series of IVT mRNA constructs were prepared that encode variations of CAR proteins with scFvs targeting CD19. Constructs contained either wild-type intracellular CD3-zeta and wild-type intracellular CD28-CD3-zeta polypeptide sequences (SEQ IDs: 18 and 66), or intracellular CD28-CD3-zeta polypeptide sequence with all Lysine residues mutated to Glutamic Acid (SEQ IDs: 56 and 65). The anti-CD19 scFv were derived from clone FMC63 (scFv1: SEQ ID NO: 87) or an anti-CD19 scFv with mutation of a Tyrosine to Alanine mutation at location 261 as described in He et al. (doi.org/10.1126/sciimmunol.adf1426) (scFv2: SEQ ID NO: 88). These constructs were otherwise identical with respect to the Cap, 5′ UTR′, 3′ UTR and poly A tail. Incorporating these mutations of Lysine residues to Glutamic Acid residues in the intracellular tail is a strategy to protect CAR with different target specificities from downregulation by their specific ligands.

For each of the mRNA constructs, CAR T cells were prepared as described in Example 1. About 24 hours after transfection, CAR T cells made from each mRNA construct were exposed to CD19⁺ Raji target cells, or no target cells by the method described in Example 1. 24 hours later the cells were evaluated for expression of CAR (using anti-Myc-Alexa 647 antibody, an extracellular tag on the CAR), by the method described in Example 1. The CAR T cells were also evaluated for their ability to perform cytotoxicity and effector function (IFN-gamma production) in response to CD19+ Raji cell lines by the method substantially described in Example 1.

CAR T cells generated using IVT mRNA including K-E mutation signaling domains SEQ ID NOs: 89 and 90 (K-E CD3-zeta and CD28), showed highest anti CD19 CAR expression in the absence of CD19 ligand. In the presence of ligand, CAR T cells generated with all constructs that contained a mutated Lysine-Glutamine CD3-zeta and CD28 signaling domains (SEQ ID NOs: 89 and 90) showed higher maintenance of CAR expression on the cell surface following exposure to Raji target cells (30%-50%) compared with that observed on comparator constructs SEQ ID NOs: 91 and 92 containing wild-type CD3-zeta (4-5%). CAR T cells generated using constructs that contained the mutant intracellular CD3 and CD28 domains had higher cytolytic abilities (Table 14). CAR T cells generated with all constructs could signal and produce Interferon-gamma upon exposure to Raji target cells at 1:2 E:T ratio (Table 15). Interferon-gamma production was highest for construct SEQ ID NO: 90, the -anti-CD19 CAR of scFv2 containing a K-E mutated CD28 and CD3-zeta signaling domains (614 pg/mL), followed by SEQ ID NO: 89, containing an anti-CD19 CAR of scFv1 and mutant CD28 and CD3-zeta signaling domains (393 pg/mL) (Table 15).

TABLE 14
Expression of anti-CD19 CAR by CAR T cells expressing CAR containing
CD28-CD3-zeta intracellular domains with wild-type sequences or mutant
sequences containing Lysines mutated to Glutamic Acid following culture
in the absence or presence of CD19⁺ Raji target cells

| | | | | CAR | CAR |
|---|---|---|---|---|---|
| | | | CAR | expression | expression |
| | | | expression | following | maintained |
| | | | in absence | exposure | following |
| | | | of target | to CD19+ | ligand |
| | | Signaling | ligand | Raji cells | exposure |
| CAR Sequence ID | Anti-CD19 scFv | Domains | (MFI) | (MFI) | (%) |
| SEQ ID NO: 91 | scFv1: SEQ ID | CD28 | 917 | 108 | 5% |
| | NO: 87 | WT, | | | |
| | | CD3-zeta | | | |
| | | WT | | | |
| SEQ ID NO: 90 | scFv1: SEQ ID | CD28 K- | 4820 | 2459 | 50% |
| | NO: 87 | E, CD3- | | | |
| | | zeta K-E | | | |
| SEQ ID NO: 92 | scFv2: SEQ ID | WT CD3, | 994 | 102 | 4% |
| | NO: 88 | WT CD28 | | | |
| SEQ ID NO: 89 | scFv2: SEQ ID | CD28 K- | 1776 | 596 | 31% |
| | NO: 88 | E, CD3- | | | |
| | | zeta K-E | | | |

TABLE 15
Cytotoxicity on CD19+ Raji cells by anti-CD19 CAR
T cells expressing CAR containing wild-type or mutant
CD28-CD3-zeta intracellular domains (effector to target: 1:2)

| Construct CAR | | | Cytotoxicity |
|---|---|---|---|
| Sequence | Anti-CD19 scFv | Signaling Domains | (%) |
| SEQ ID NO: 91 | scFv1: SEQ ID NO: 87 | CD28 WT, CD3-zeta | 22 |
| | | WT | |
| SEQ ID NO: 90 | scFv1: SEQ ID NO: 87 | CD28 K-E, CD3-zeta | 26 |
| | | K-E | |
| SEQ ID NO: 92 | scFv2: SEQ ID NO: 88 | WT CD3, WT CD28 | 10 |
| SEQ ID NO: 89 | scFv2: SEQ ID NO: 88 | CD28 K-E, CD3-zeta | 17 |
| | | K-E | |

TABLE 16
Cytokine (Interferon-gamma) production by anti-CD19 CAR T cells
expressing CAR containing wild-type or mutant CD28-CD3-zeta intracellular
domains following exposure to CD19⁺ Raji cells

| Construct CAR | | | Interferon- |
|---|---|---|---|
| Sequence | scFv | Signaling Domains | gamma (pg/mL) |
| SEQ ID NO: 91 | FMC63: Anti-CD19 | CD28 WT, CD3-zeta | 233 |
| | SEQ ID NO: 87 | WT | |
| SEQ ID NO: 90 | FMC63: Anti-CD19 | CD28 K-E, CD3-zeta | 393 |
| | SEQ ID NO: 87 | K-E | |
| SEQ ID NO: 92 | FMC63 Y261A: Anti- | WT CD3, WT CD28 | 144 |
| | CD19 SEQ ID NO: 88 | | |
| SEQ ID NO: 89 | FMC63 Y261A: Anti- | CD28 K-E, CD3-zeta | 614 |
| | CD19 SEQ ID NO: 88 | K-E | |

In summary, CAR constructs containing CD28-CD3-zeta intracellular signaling domains that were resistant to ligand-dependent downregulation by mutation of Lysine amino acids, were successfully generated using scFvs targeting CD19. The mutated anti-CD19 CAR T cells performed CAR-dependent cytokine production and cytolytic activity. CAR T cells generated with CAR containing K-E mutations in the intracellular domain showed resistance to ligand-mediated downregulation and superior effector function (cytokine) in the presence of CD19⁺ Raji cells, compared with their wild-type counterparts.

### Example 13

This example describes an experiment to test how tumor burden can be controlled in vivo using CAR T cells generated with IVT mRNA encoding BCMA CAR that has been rendered resistant to ligand-induced downregulation by mutation of Lysine residues in the intracellular signaling domain to Glutamic Acid. These mRNA CAR constructs comprise the sequences of (SEQ ID NOs: 17 and 67). These mRNA constructs encode CAR proteins that comprise, respectively, the sequences of (SEQ ID NOs: 5 and 68).

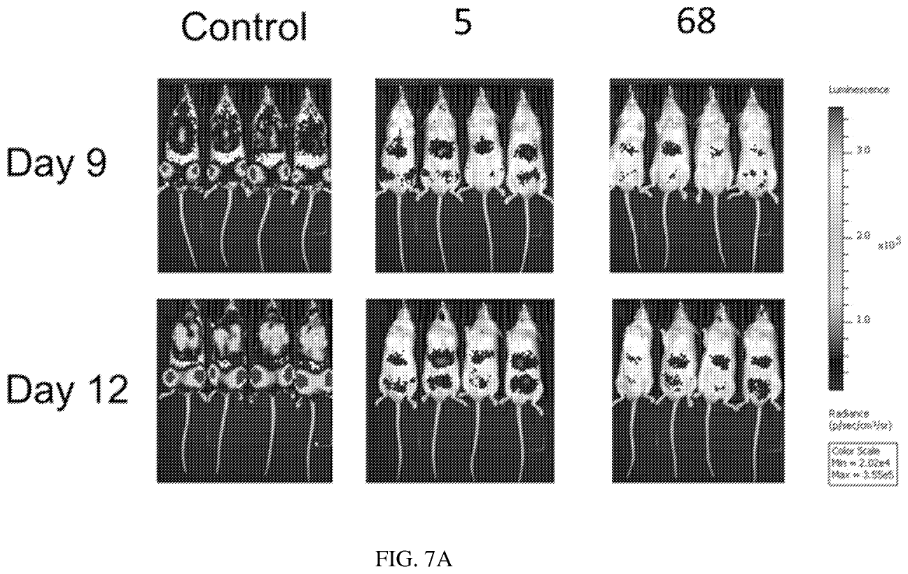

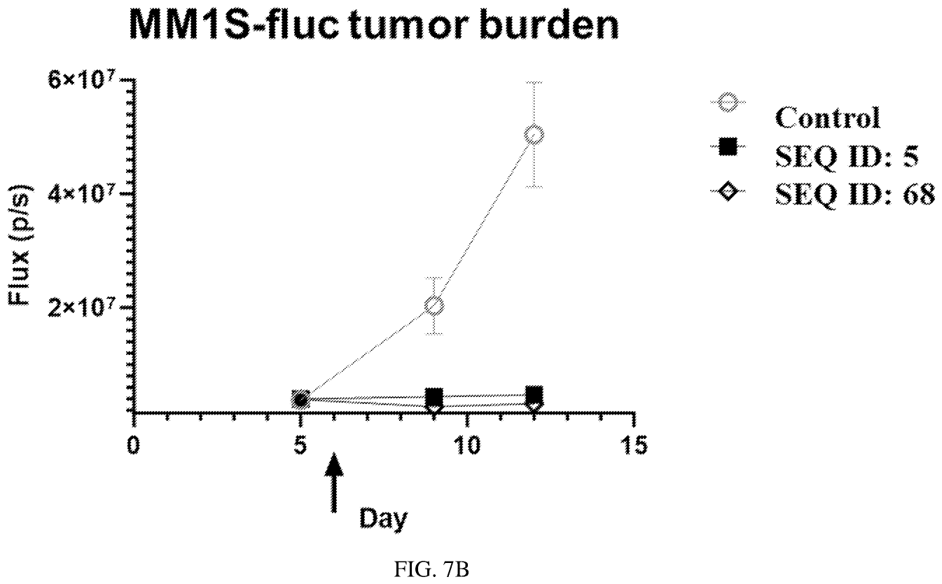

CAR T cells were prepared by transfection of the mRNA CAR constructs into human CD8+ T cells, as described in Example 1, with the exception that the endogenous T cell receptor was genetically removed from the cells by CRISPR/Cas engineering. Negative control cells that did not include the IVT mRNA were prepared from the same batch of TCR-knockout CD8⁺ T cells. Immunocompromised Nod-Scid IL2R-gamma knockout (NSG) mice were inoculated with 2 million MM1S-fluc human multiple myeloma tumor cells on Day 0. Tumor burden was monitored by serial bioluminescence imaging (BLI). On Day 5 mice were randomized into treatment groups. On Day 6 mice were administered i.v. with 2 million control CD8⁺ T Cells, or 2 million CAR T Cells engineered to express a CAR of SEQ ID NO: 5, or 2 million CAR T cells engineered to express a CAR of SEQ ID NO: 68. Tumor burden was measured on Days 9 and 12. Tumor burden as measured by BLI (photons/second) on Day 9 was 4.7-fold lower in mice administered CAR cells expressing SEQ ID NO: 5 or 7.9-fold lower in mice administered with CAR cells expressing SEQ ID NO: 68 than in mice administered control CD8⁺ T cells. On Day 12, 10.8-fold (SEQ ID NO: 5) and 16.0-fold (SEQ ID NO: 68) reductions in BLI were observed compared with control mice. All comparisons were highly significant (p<0.001; two-way ANOVA on log-transformed data). FIGS. 7A-7B and Table 17 show BLI data from individual mice (FIG. 7A) and summary statistics (FIG. 7B).

TABLE 17
Bioluminescence Imaging of MM1S-fluc tumor burden
in NSG mice treated with control CD8+ T cells
or cells expressing anti-BCMA CAR containing mutations
to Lysines in the intracellular signaling domains.

| CAR expressed | Signaling | Day 9 Flux (p/s) | Day 12 Flux (p/s) |
|---|---|---|---|
| by CD8+ T cells | Domains | (mean of n = 4) | (mean of n = 4) |
| none | Not applicable | 20,325,000 | 50,425,000 |
| SEQ ID NO: 5 | CD3-zeta K-E | 4,330,000 | 4,682,500 |
| SEQ ID NO: 68 | CD28 K-E, | 2,575,000 | 3,142,500 |
| | CD3-zeta K-E | | |

In summary, CAR T constructs of sequences SEQ ID NO: 5 and SEQ ID NO: 68, containing either CD3-zeta alone or CD28-CD3-zeta signaling domains with Lysine residues mutated to Glutamic Acid, produced CART cells that robustly controlled BCMA⁺ Tumor burden in vivo.

### Example 14

This example describes an experiment to evaluate the activity and duration of CAR T cells generated with the IVT mRNA constructs of the present disclosure in vivo in a mouse model of multiple myeloma.

CAR T cells were prepared by transfection of the IVT mRNA CAR constructs into human CD8⁺ T cells, as described in Example 1. CAR T cells were generated using IVT mRNA encoding sequence SEQ ID NO: 68, that contains a CD28-CD3-zeta intracellular signaling domain in which Lysine residues were mutated to Glutamic Acid (SEQ ID NO: 65), and an IVT mRNA encoding a control CAR that contains a wild-type CD28-CD3-zeta intracellular signaling domain SEQ ID NO: 66. Negative control cells that did not include the IVT mRNA were prepared from the same batch of CD8⁺ T cells. Immunocompromised Nod-Scid IL2R-gamma knockout (NSG) mice were inoculated with 2 million MM1S-fluc human multiple myeloma tumor cells on Day 0. Tumor burden was monitored by serial bioluminescence imaging (BLI). On Day 8 mice were randomized into treatment groups. On Day 9 mice were administered i.v. with either 2 million or 12.5 million of the following cells: control CD8⁺ T Cells, CAR T Cells engineered to express a K-E mutated CAR SEQ ID NO: 65, CAR T cells engineered to express a wild-type CAR of SEQ ID NO: 66. Tumor burden was measured on Days 11, 16, and 21. Pre-selected individual mice were harvested on Days 10, 11, and 14 for analysis of CAR expression by transferred cells in the blood, spleen, and bone marrow.

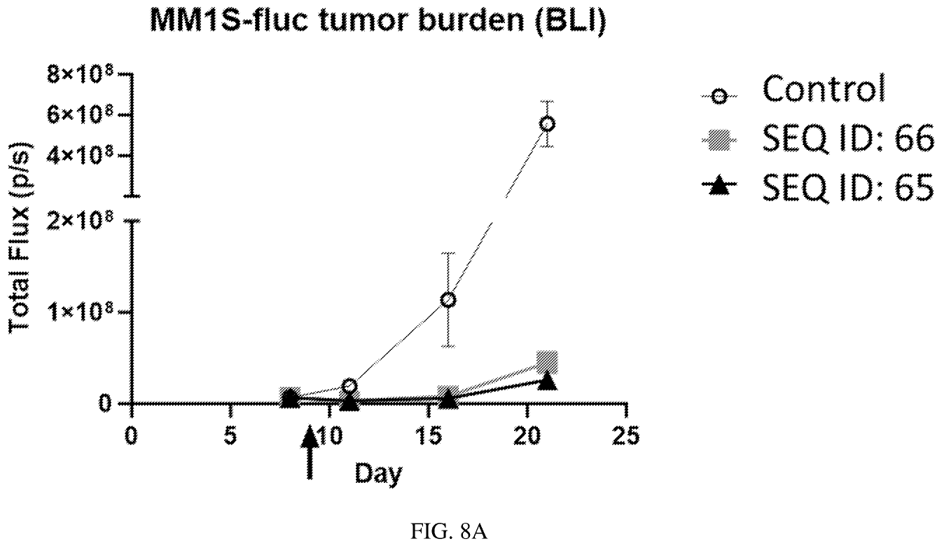

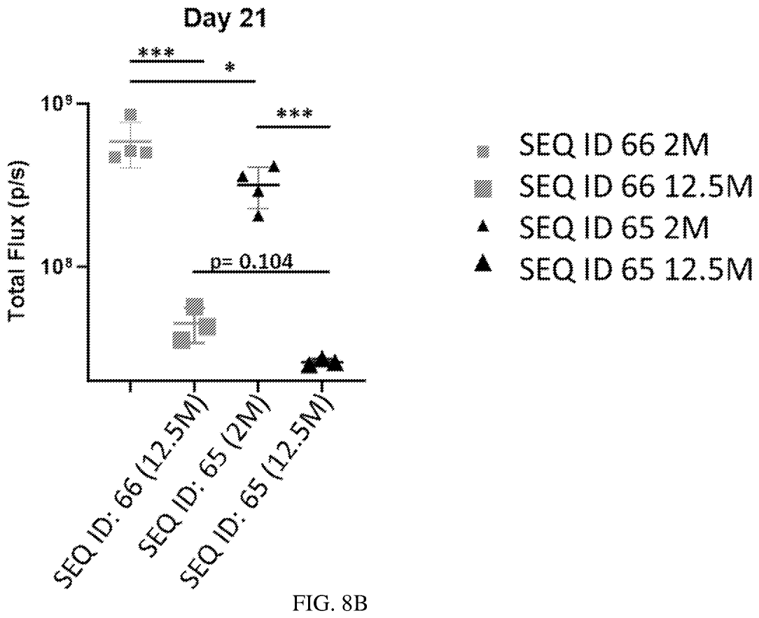

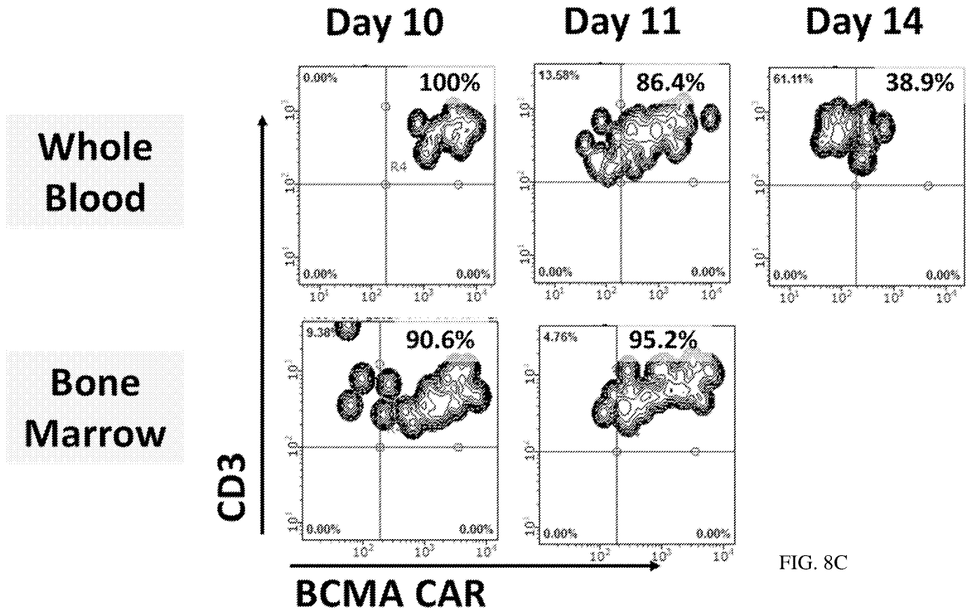

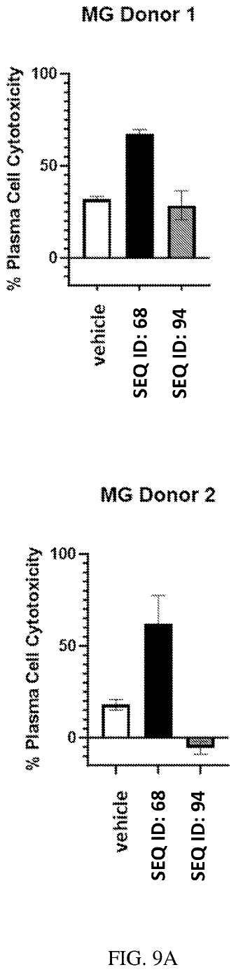

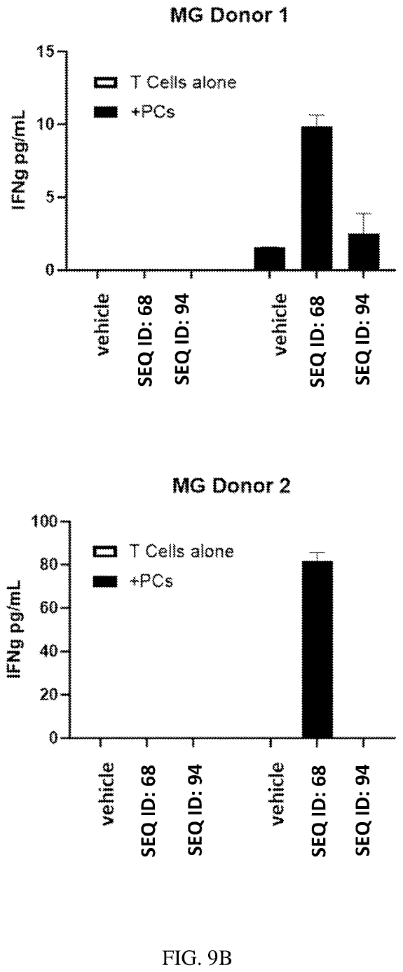

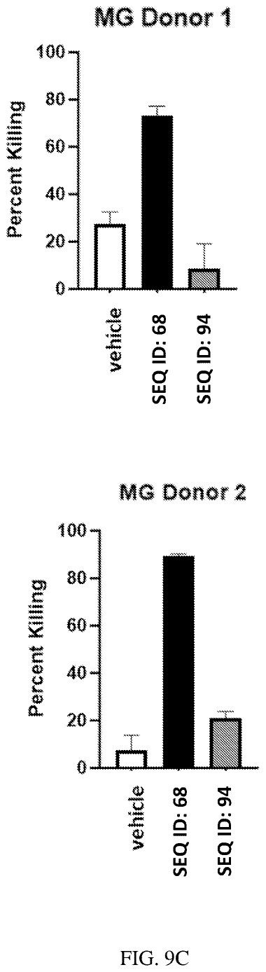

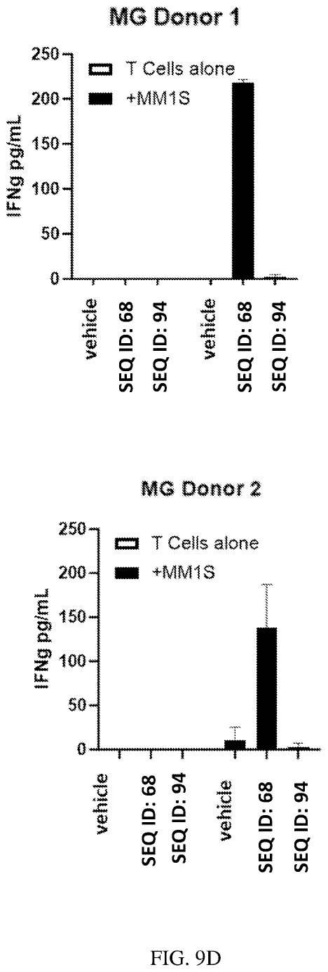

A single administration of 12.5 million anti-BCMA CAR T cells generated with IVT mRNA constructs containing K-E-mutated SEQ ID NO: 65 or wild-type SEQ ID NO: 66 anti-BCMA CAR on Day 9 controlled tumor burden compared with control CD8⁺ T cells (21.4-fold and 12.4-fold reductions in tumor burden at Day 21, respectively) (FIG. 8A). Control of tumor burden was dose dependent (FIG. 8B). CAR T cells generated with the SEQ ID NO: 65 CAR construct, containing a CD28-CD3-zeta signaling domain with Lysine residues mutated to Glutamic Acid, showed improved control of tumor burden than the SEQ ID NO: 66 CAR T cells containing a wild-type CD28-CD3-zeta signaling domain (p<0.05 for 2 million cells; FIG. 8B).

Analysis of whole blood and bone marrow from mice administered with anti-BCMA CAR T cells containing CAR including a K-E-mutated SEQ ID NO: 65 identified human CAR T cells expressing anti-BCMA CAR out to Day 14 (FIG. 8C).

In summary, CAR T constructs of sequences SEQ ID NO: 68, containing a CD28-CD3-zeta signaling domain with Lysine residues mutated to Glutamic Acid, produced CART cells that robustly controlled BCMA⁺ Tumor burden in vivo and demonstrated durable expression of CAR in vivo.

### Example 15

This example describes an experiment to test the capability to generate anti-BCMA CAR T cells expressing the downmodulation resistant CAR proteins of the present disclosure in autoimmune disease subject T cells.

Anti-BCMA CAR T cells from two myasthenia gravis disease donors were prepared according to the methods of Example 1 using the CAR receptors generated with SEQ ID NO: 68, and SEQ ID NO: 94 (anti-PSMA CAR as negative control CAR). Vehicle EP was set as a negative EP control. Autologous plasma cells were isolated from the same two myasthenia gravis disease donors. 24 hours after transfection. CAR-T cells were co-cultured with either autologous plasma cells, or malignant MM1S-GFP cells. CAR-T cells generated from MG donors were evaluated for their CAR expression (MFI) and cytolytic activities (i.e. percent cytotoxicity, Interferon-gamma).

CAR T cells generated with the CAR of the present disclosure expressed anti-BCMA CAR. Donor variation observed with CAR expression after 24 hours post-EP (MFI=1887 vs 3766). CAR T cells could effectively kill the autologous plasma cells in vitro, as measured by cytotoxicity of autologous plasma cells differentiated from MG patients (E:T=1:2, 67.5% cytotoxicity for Donor 1, and 62% cytotoxicity for donor 2), whereas neither vehicle transfected T cells or anti-PSMA control CAR yielded lower killing percentage (32%, 18% for donor 1, 18%, −5.5% for donor 2). Donor variation was also observed in Interferon-gamma cytokine production post cytotoxicity (9.82 pg/mL for donor 1 and 81.68 pg/mL for donor 2). Functionality of MG donor CAR T cells was also evaluated using malignant MM1S-GFP cell line. Effective killing of MM1S-GFP cells was observed for both donors (73% for donor 1, 89.5% for donor 2), with cytokine production (217.71 pg/mL for donor 1, 138.38 pg/mL for donor 2). No significant killing or cytokine was observed in control CAR T groups.

TABLE 18
Expression of anti-BCMA CAR containing CD28-figCD3-
zeta intracellular domains with mutant sequences
containing Lysines mutated to Glutamic Acid,
and anti-PSMA CAR as a control CAR.

| | | SEQ ID NO: | SEQ ID NO: |
|---|---|---|---|
| Donor | Control MFI | 68 MFI | 94 MFI |
| MG Donor 1 | 49 | 1,887 | 592 |
| MG Donor 2 | 53 | 3,766 | 651 |

TABLE 19
Functionality of MG donor CAR T cells generated using downmodulation
resistant CAR containing mutant CD28-CD3-zeta intracellular signaling
domains: against autologous plasma cells (n = 2).

| | Construct | Percent killing ± | Interferon-gamma |
|---|---|---|---|
| Donor | (seqID) | SD (%) | (pg/mL) |
| MG donor 1 | Control | 32 ± 1 | 1.6 ± 0.0 |
| | SEQ ID NO: 68 | 67.5 ± 1.5 | 9.8 ± 0.6 |
| | SEQ ID NO: 94 | 28.5 ± 5.5 | 2.5 ± 1.0 |
| MG donor 2 | Control | 18 ± 2 | 0 |
| | SEQ ID NO: 68 | 62 ± 11 | 81.7 ± 2.8 |
| | SEQ ID NO: 94 | −5.5 ± 2.5 | 0 |

TABLE 20
Functionality of MG donor CAR T cells generated using downmodulation
resistant CAR containing mutant CD28-CD3-zeta intracellular signaling
domains: against malignant MM1S-GFP cells (n = 2).

| | Construct | Percent killing ± | Interferon-gamma |
|---|---|---|---|
| Donor | (seqID) | SD (%) | (pg/mL) |
| MG donor 1 | Control | 27.5 ± 3.5 | 0 |
| | SEQ ID NO: 68 | 73 ± 3 | 217.7 ± 2.9 |
| | SEQ ID NO: 94 | 8.5 ± 7.5 | 2.0 ± 2.0 |
| MG donor 2 | Control | 7.5 ± 4.5 | 10.5 ± 10.5 |
| | SEQ ID NO: 68 | 89.5 ± 0.5 | 138.4 ± 34.4 |
| | SEQ ID NO: 94 | 21 ± 2 | 3.1 ± 3.1 |

In summary, downmodulation resistant signaling domain containing CAR T cells can be generated using autoimmune disease patient CD8 T cells. These CAR-T cells can robustly express anti-BCMA CAR and carry cytolytic activities on either autologous plasma cells or malignant MM1S cells with cytokine production.

### Example 16

This example describes an experiment to further increase CAR expression and CAR T cell functionality by incorporating lower affinity scFv onto the downmodulation resistant signaling domains. Mutating Lysine residues to Glutamic Acid residues in the intracellular domain is a strategy to protect CAR with different target specificities from downregulation by their specific ligands. Incorporating lower affinity scFv strengthens this strategy to further increase the CAR retention post target exposure.

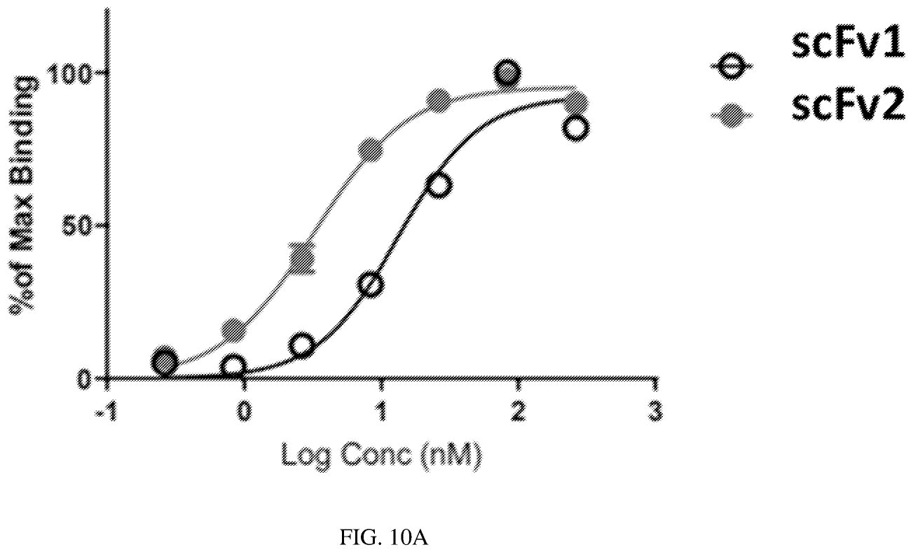

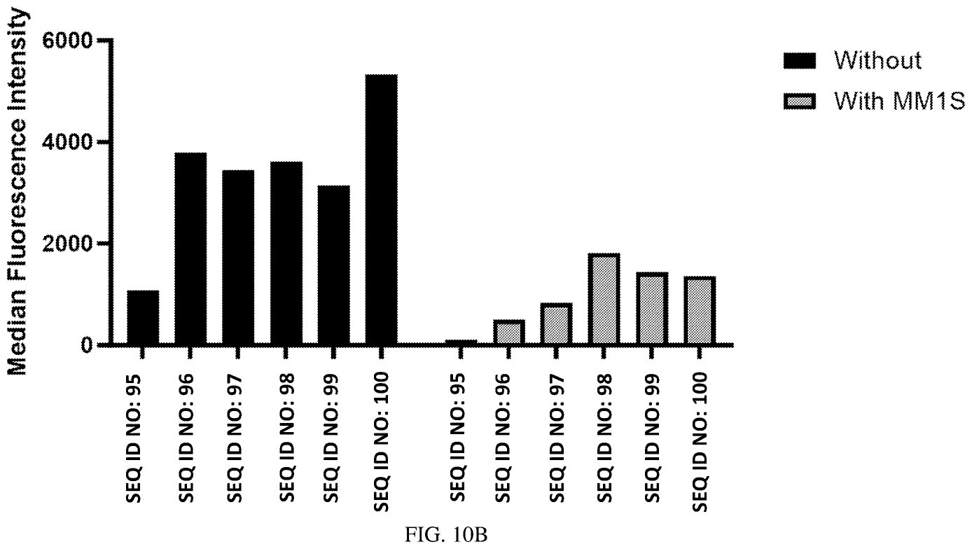

A series of IVT mRNA constructs were prepared that encode variations of CAR proteins with two scFv targeting BCMA. scFv1 with apparent affinity KD 3.2 nM, and scFv2 with apparent affinity KD 13.3 nM (FIG. 10A). Constructs contained either wild-type intracellular CD3-zeta and wild-type intracellular CD28-CD3-zeta polypeptide sequences or intracellular CD28-CD3-zeta polypeptide sequence with all Lysine residues mutated to Glutamic Acid. These constructs were otherwise identical with respect to the Cap, 5′ UTR, 3′ UTR and poly A tail. For each of the mRNA constructs, CAR T cells were prepared as described in Example 1. About 24 hours after transfection, CAR T cells made from each mRNA construct were exposed to BCMA+ target cells (MM1S), or no target cells by the method described in Example 1. 24 hours later the cells were evaluated for expression of CAR, by the method described in Example 1. The CAR T cells were also evaluated for their ability to perform cytotoxicity in response to BCMA⁺ cell lines by the method substantially described in Example 1.

All CAR T cells generated showed highest anti-BCMA expression in the absence of BCMA ligand, with a CAR containing scFv1 and mutated intracellular signaling domains CD3-zeta K-E, comprising SEQ ID NO: 101, and encoded by RNA SEQ ID: 98, showing the highest expression (MFI=5336). In the presence of BCMA ligand, CAR T cells generated with all constructs that contained a mutated Lysine-Glutamine CD3-zeta signaling domain showed higher CAR retention on the cell surface following exposure to BCMA target cells (26%, 46%, 24%, 50%) compared with that on the constructs containing wild-type CD3-zeta (10% and 13%). Within these intracellular signaling domain mutant CAR T cells, cells containing the lower affinity scFv2 showed higher maintenance of CAR expression post MM1S exposure (46% and 50%) compared to their higher affinity scFv1 counterparts (24% and 26%). CAR T cells generated with SEQ ID NO: 98 showed the highest maintenance of CAR expression following exposure to BCMA⁺ target cells. CAR T cells generated using constructs that contained the lower affinity scFv2 (SEQ ID: 98 encoding CAR of SEQ ID: 101, and SEQ ID: 99 encoding CAR of SEQ ID: 102) also had higher cytolytic abilities (94% and 91%) compared to higher affinity scFv1 CAR T cells (40%-58%).

TABLE 21
Expression of anti-CD19 CAR by CAR T cells expressing
CAR containing CD28-CD3-zeta intracellular domains
with wild-type sequences or mutant sequences containing
Lysines mutated to Glutamic Acid following culture
in the absence or presence of BCMA + MM1S cells.

| | | | MFI1 | | |
|---|---|---|---|---|---|
| | | Signaling | (Target | MFI2 | |
| SEQ ID NO | scFv | domain | exposed) | (media) | % retention |
| 95 | scFv1 | CD3-zeta WT | 103 | 1089 | 10 |
| 96 | scFv1 | CD28 WT, | 500 | 3790 | 13 |
| | | CD3-zeta WT | | | |
| 97 | scFv1 | CD28 K-E, | 831 | 3440 | 24 |
| | | CD3-zeta K-E | | | |
| 98 | scFv2 | CD3-zeta K-E | 1815 | 3610 | 50 |
| 99 | scFv2 | CD28 K-E, | 1440 | 3148 | 46 |
| | | CD3-zeta K-E | | | |
| 100 | scFv1 | CD3-zeta K-E | 1363 | 5336 | 26 |

TABLE 22
48-hour cytotoxicity on MM1S GFP cells by anti-CD19
CAR T cells expressing CAR containing wild-type
or mutant CD28-CD3-zeta intracellular domains and
different affinity scFv (effector to target: 1:4)

| Constructs | scFv | Signaling domain | MM1S killing (1:4) % |
|---|---|---|---|
| 95 | scFv1 | CD3-zeta WT | 40 ± 3 |
| 96 | scFv1 | CD28 WT, CD3-zeta WT | 44 ± 7 |
| 97 | scFv1 | CD28 K-E, CD3-zeta K-E | 42 ± 1 |
| 98 | scFv2 | CD3-zeta K-E | 94 ± 1 |
| 99 | scFv2 | CD28 K-E, CD3-zeta K-E | 91 ± 1 |
| 100 | scFv1 | CD3-zeta K-E | 58 ± 1 |

In summary, in addition to mutations in the CD28 and CD3-zeta signaling domains, modulation of the affinity of the scFv for target shows improvement in maintenance of CAR function using RNA CAR.

### Example 16

The various substitution of the disclosure and the location of the respective lysines is provided below:

| SEQ ID | | Number of | |
|---|---|---|---|
| NO: | Mutation | AA Mutated | Lysine location |
| 2 | K to R | 9 | 353, 387, 398, 403, 415, 417, 428, 435, 449 |
| 3 | K to A | 9 | 353, 387, 398, 403, 415, 417, 428, 435, 449 |
| 19 | K to A | 1 | 3 |
| 20 | K to A | 1 | 37 |
| 21 | K to A | 1 | 48 |
| 22 | K to A | 1 | 53 |
| 23 | K to A | 1 | 65 |
| 24 | K to A | 1 | 67 |
| 25 | K to A | 1 | 78 |
| 26 | K to A | 1 | 85 |
| 27 | K to A | 1 | 99 |
| 28 | K to A | 7 | 37, 53, 65, 67, 78, 85, 99 |
| 29 | K to A | 6 | 37, 65, 67, 78, 85, 99 |
| 30 | K to A | 2 | 3, 48 |
| 31 | K to A | 7 | 3, 37, 48, 53, 78, 85, 99 |
| 32 | K to A | 7 | 3, 37, 48, 53, 65, 67, 99 |
| 33 | K to A | 7 | 3, 48, 53, 65, 67, 78, 85 |
| 34 | K to A | 8 | 37, 48, 53, 65, 67, 78, 85, 99 |
| 35 | K to A | 8 | 3, 37, 53, 65, 67, 78, 85, 99 |
| 36 | K to A | 8 | 3, 37, 48, 53, 65, 78, 85, 99 |
| 37 | K to A | 8 | 3, 37, 48, 53, 65, 67, 78, 99 |
| 38 | K to A | 8 | 3, 48, 53, 65, 67, 78, 85, 99 |
| 39 | K to A | 8 | 3, 37, 48, 65, 67, 78, 85, 99 |
| 40 | K to A | 8 | 3, 37, 48, 53, 67, 78, 85, 99 |
| 41 | K to A | 8 | 3, 37, 48, 53, 65, 67, 85, 99 |
| 42 | K to A | 8 | 3, 37, 48, 53, 65, 67, 78, 85 |
| 43 | K to A | 9 | 3, 37, 48, 53, 65, 67, 78, 85, 99 |
| 44 | K to V | 9 | 3, 37, 48, 53, 65, 67, 78, 85, 100 |
| 45 | K to I | 9 | 3, 37, 48, 53, 65, 67, 78, 85, 101 |
| 46 | K to L | 9 | 3, 37, 48, 53, 65, 67, 78, 85, 102 |
| 47 | K to M | 9 | 3, 37, 48, 53, 65, 67, 78, 85, 103 |
| 48 | K to F | 9 | 3, 37, 48, 53, 65, 67, 78, 85, 104 |
| 49 | K to Y | 9 | 3, 37, 48, 53, 65, 67, 78, 85, 105 |
| 50 | K to S | 9 | 3, 37, 48, 53, 65, 67, 78, 85, 106 |
| 51 | K to T | 9 | 3, 37, 48, 53, 65, 67, 78, 85, 107 |
| 52 | K to N | 9 | 3, 37, 48, 53, 65, 67, 78, 85, 108 |
| 53 | K to Q | 9 | 3, 37, 48, 53, 65, 67, 78, 85, 109 |
| 54 | K to H | 9 | 3, 37, 48, 53, 65, 67, 78, 85, 110 |
| 55 | K to D | 9 | 3, 37, 48, 53, 65, 67, 78, 85, 111 |
| 56 | K to E | 9 | 3, 37, 48, 53, 65, 67, 78, 85, 112 |
| 57 | K to A | 9 | 44, 78, 89, 94, 106, 108, 119, 126, 140 |
| 58 | K to A | 11 | 3, 25, 44, 78, 89, 94, 106, 108, 119, 126, 140 |
| 59 | K to A | 9 | 44, 78, 89, 94, 106, 108, 119, 126, 140 |
| 60 | K to A | 13 | 1, 5, 6, 12, 45, 79, 90, 95, 107, 109, 120, 127, |
| | | | 141 |
| 62 | K to E CD28 | 11 | K to E: 3, 25; |
| | domain; | | K to A: 44, 78, 89, 94, 106, 108, 119, 126, |
| | K to A CD3Z | | 140 |
| | domain | | |
| 63 | K to E | 9 | 44, 78, 89, 94, 106, 108, 119, 126, 140 |
| 64 | K to A CD28 | 11 | K to A: 3, 25; |
| | domain; | | K to E: 44, 78, 89, 94, 106, 108, 119, 126, |
| | K to E CD3Z | | 140 |
| | domain | | |
| 65 | K to E CD28 | 11 | K to E: 3, 25; |
| | domain; | | K to E: 44, 78, 89, 94, 106, 108, 119, 126, |
| | K to E CD3Z | | 140 |
| | domain | | |
| 68 | K to E | 11 | 353, 375, 394, 428, 439, 444, 456, |
| | | | 458, 469, 476, 490 |
| 69 | 8 K to E; 1 K | 9 | 3 (A), 37, 48, 53, 65, 67, 78, 85, 99 |
| | to A | | |
| 70 | 8 K to E; 1 K | 9 | 3, 37 (A), 48, 53, 65, 67, 78, 85, 99 |
| | to A | | |
| 71 | 8 K to E; 1 K | 9 | 3, 37, 48 (A), 53, 65, 67, 78, 85, 99 |
| | to A | | |
| 72 | 8 K to E; 1 K | 9 | 3, 37, 48, 53 (A), 65, 67, 78, 85, 99 |
| | to A | | |
| 73 | 8 K to E; 1 K | 9 | 3, 37, 48, 53, 65 (A), 67, 78, 85, 99 |
| | to A | | |
| 74 | 8 K to E; 1 K | 9 | 3, 37, 48, 53, 65, 67 (A), 78, 85, 99 |
| | to A | | |
| 75 | 8 K to E; 1 K | 9 | 3, 37, 48, 53, 65, 67, 78 (A), 85, 99 |
| | to A | | |
| 76 | 8 K to E; 1 K | 9 | 3, 37, 48, 53, 65, 67, 78, 85 (A), 99 |
| | to A | | |
| 77 | 8 K to E; 1 K | 9 | 3, 37, 48, 53, 65, 67, 78, 85, 99 (A) |
| | to A | | |
| 78 | 1 K to E: 8 K to | 9 | 3 (E), 37, 48, 53, 65, 67, 78, 85, 99 |
| | A | | |
| 79 | 1 K to E: 8 K to | 9 | 3, 37 (E), 48, 53, 65, 67, 78, 85, 99 |
| | A | | |
| 80 | 1 K to E: 8 K to | 9 | 3, 37, 48 (E), 53, 65, 67, 78, 85, 99 |
| | A | | |
| 81 | 1 K to E: 8 K to | 9 | 3, 37, 48, 53 (E), 65, 67, 78, 85, 99 |
| | A | | |
| 82 | 1 K to E: 8 K to | 9 | 3, 37, 48, 53, 65 (E), 67, 78, 85, 99 |
| | A | | |
| 83 | 1 K to E: 8 K to | 9 | 3, 37, 48, 53, 65, 67 (E), 78, 85, 99 |
| | A | | |
| 84 | 1 K to E: 8 K to | 9 | 3, 37, 48, 53, 65, 67, 78 (E), 85, 99 |
| | A | | |
| 85 | 1 K to E: 8 K to | 9 | 3, 37, 48, 53, 65, 67, 78, 85 (E), 99 |
| | A | | |
| 86 | 1 K to E: 8 K to | 9 | 3, 37, 48, 53, 65, 67, 78, 85, 99 (E) |
| | A | | |

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which two or more members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the present disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the present disclosure, can be excluded from any one or more claims. For purposes of brevity, all of the embodiments in which one or more elements, features, purposes, or aspects is excluded are not set forth explicitly herein.

### EMBODIMENTS

The following embodiments are within the scope of the present disclosure.

1. A protein that is capable of intracellular signaling comprising a CD3-zeta intracellular domain, wherein the CD3-zeta intracellular domain is 80% identical to SEQ ID NO: 18, and wherein at least six lysine amino acids of SEQ ID NO: 18 are substituted by an amino acid independently selected from the group consisting of: alanine, aspartate, asparagine, glutamate, glutamine, histidine, leucine, methionine, serine, threonine, and valine.
2. The protein of embodiment 1, where the CD3-zeta intracellular domain is 100% identical to SEQ ID NO:18 except for the substituted lysine amino acids.
3. The protein of embodiment 1 or 2, wherein at least seven lysine amino acids of SEQ ID NO: 18 are substituted by an amino acid independently selected from the group consisting of: alanine, aspartate, asparagine, glutamate, glutamine, histidine, leucine, methionine, serine, threonine, and valine.
4. The protein of any one of embodiments 1-3, wherein at least eight lysine amino acids of SEQ ID NO: 18 are substituted by an amino acid independently selected from the group consisting of: alanine, aspartate, asparagine, glutamate, glutamine, histidine, leucine, methionine, serine, threonine, and valine.
5. The protein of any one of embodiments 1-4, wherein the nine lysine amino acids of SEQ ID NO: 18 are substituted by an amino acid independently selected from the group consisting of: alanine, aspartate, asparagine, glutamate, glutamine, histidine, leucine, methionine, serine, threonine, and valine.
6. The protein of any one of embodiments 1-5, wherein the at least six lysine amino acids of SEQ ID NO: 18 are substituted by an amino acid independently selected from the group consisting of: alanine, aspartate, and glutamate.
7. The protein of any one of embodiments 1-6, wherein at least seven lysine amino acids of SEQ ID NO: 18 are substituted by an amino acid independently selected from the group consisting of: alanine, aspartate, and glutamate.
8. The protein of any one of embodiments 1-7, wherein at least eight lysine amino acids of SEQ ID NO: 18 are substituted by an amino acid independently selected from the group consisting of: alanine, aspartate, and glutamate.
9. The protein of any one of embodiments 1-8, wherein the nine lysine amino acids of SEQ ID NO: 18 is substituted by an amino acid independently selected from the group consisting of: alanine, aspartate, and glutamate.
10. A protein comprising a CD3-zeta intracellular domain, wherein the CD3-zeta intracellular domain is 80% identical to SEQ ID NO:18, wherein at least six lysine amino acids of SEQ ID NO: 18 is either (a) deleted or (b) substituted by an amino acid independently selected from the group consisting of: alanine, aspartate, and glutamate.
11. The protein of embodiments 10, where the CD3-zeta intracellular domain is 100% identical to SEQ ID NO:18 except for the deleted or substituted lysine amino acids.
12. The protein of embodiments 10 or 11, wherein at least seven lysine amino acids of SEQ ID NO: 18 is either (a) deleted or (b) substituted by an amino acid independently selected from the group consisting of: alanine, aspartate, and glutamate.
13. The protein of any one of embodiments 10-12, wherein at least eight lysine amino acids of SEQ ID NO: 18 is either (a) deleted or (b) substituted by an amino acid independently selected from the group consisting of: alanine, aspartate, and glutamate.
14. The protein of any one of embodiments 10-13, wherein the nine lysine amino acids of SEQ ID NO: 18 is either (a) deleted or (b) substituted by an amino acid independently selected from the group consisting of: alanine, aspartate, and glutamate.
15. The protein of any one of embodiments 1-14, wherein at least six lysine amino acids of SEQ ID NO: 18 are substituted by alanine.
16. The protein of claim any one of embodiments 1-15, wherein at least seven lysine amino acids of SEQ ID NO: 18 are substituted by alanine.
17. The protein of any one of embodiments 1-16, wherein at least eight lysine amino acids of SEQ ID NO: 18 are substituted by alanine.
18. The protein of any one of embodiments 1-17, wherein the nine lysine amino acids of SEQ ID NO: 18 are substituted by alanine.
19. The protein of any one of embodiments 1-14, wherein at least six lysine amino acids of SEQ ID NO: 18 are substituted by aspartate.
20. The protein of any one of embodiments 1-14 or 19, wherein at least seven lysine amino acids of SEQ ID NO: 18 are substituted by aspartate.
21. The protein of any one of embodiments 1-14, 19, or 20, wherein at least eight lysine amino acids of SEQ ID NO: 18 are substituted by aspartate.
22. The protein of any one of embodiments 1-14 or 19-21, wherein the nine lysine amino acids of SEQ ID NO: 18 are substituted by aspartate.
23. The protein of any one of embodiments 1-14, wherein at least six lysine amino acids of SEQ ID NO: 18 are substituted by glutamate.
24. The protein of any one of embodiments 1-14 or 23, wherein at least seven lysine amino acids of SEQ ID NO: 18 is substituted by glutamate.
25. The protein of any one of embodiments 1-14, 23, or 24, wherein at least eight lysine amino acids of SEQ ID NO: 18 are substituted by glutamate.
26. The protein of any one of embodiments 1-14 or 23-25, wherein the nine lysine amino acids of SEQ ID NO: 18 are substituted by glutamate.
27. A protein of any of embodiments 1-26 comprising an amino acid sequence that is 80% identical to any one of SEQ ID NOs: 2-7, 19-66, and 68-86.
28. The protein of embodiment 1 comprising an amino acid sequence that is 80% identical to any one of SEQ ID NOs: 2-7, 19-66, and 68-86.
29. The protein of any one of embodiments 1-28, wherein the protein is Chimeric Antigen Receptor (CAR).
30. A nucleic acid encoding the protein of any one of embodiments 1-29.
31. The nucleic acid of embodiment 30, wherein the nucleic acid comprises ribonucleic acid.
32. The nucleic acid of embodiment 30 or 31, wherein the nucleic acid is ribonucleic acid.
33. A cell comprising the nucleic acid of any one of embodiments 30-32.
34. The cell of embodiment 33, wherein the cell is a human cell.
35. A viral vector adapted to express the protein of any one of embodiments 1-29.
36. A cell modified to express the protein of any one of embodiments 1-29.
37. The cell of embodiment 36, wherein the cell is a human cell.
38. A method for producing a cell therapy, the method comprising combining a cell with a nucleic acid encoding the protein of any one of embodiments 1-29.
39. The method of embodiment 38, wherein the cell is a human cell.
40. The method of embodiment 39, wherein the human cell is a T cell.
41. The method of embodiment 39, wherein the human cell is a CD3+ cell.
42. The method of embodiment 39, wherein the human cell is a CD8+ cell.
43. The method of embodiment 39, wherein the human cell is a CD4+ cell.
44. The method of embodiment 39, wherein the human cell is a NK cell.
45. The method of embodiment 39, wherein the human cell is a stem cell.
46. The method of embodiment 45, wherein the stem cell is a hematopoietic stem cell.
47. The method of embodiment 45, wherein the stem cell is a mesenchymal stem cell.
48. A kit comprising one or more of the proteins of any one of embodiments 1-29, a nucleic acid of any one of embodiments 30-32, a cell of embodiments 33, 34, 36, or 37, or the vector of embodiment 35.

### Sequences

The following amino acid (AA) or nucleotide (nt) sequences are referenced herein. For the amino acid sequences SEQ ID NOs: 1-5 and the nucleic acid sequences SEQ ID NOS: 6-10 provided below, the signal peptide-scFv-CD8 sequence or the nucleic acid encoding the signal peptide-scFv-CD8 sequence is indicated in bold, and the signaling domain or the nucleic acid encoding the signaling domain is indicated by underlining. Double underlined letters indicated muted amino acids. Except where otherwise noted, nucleic acid sequences set forth below and in the instant application may recite “T”s in a representative DNA sequence but where the sequence represents RNA, the “T”s would be substituted for “U”s. Thus, any of the DNAs disclosed and identified by a particular sequence herein also discloses the corresponding RNA sequence where each “T” of the DNA sequence is substituted with “U”:

(AA, Polypeptide Anti-BCMA CAR containing WT CD3z only)
SEQ ID NO: 1
MALPVTALLLPLALLLHAARPDIVLTQSPASLAVSPGQRATITCRASESVTILGSHLIHWYQQKPGQPPKL
LINLASNVNTGVPARFSGSGSGTDFTLTISSVEPEDTANYYCLQSRTLPRTFGQGTKVEIKGSTSGSGKPG
SGEGSTKGQIQLVQSGPELKKPGGSVKISCKASGYTFTDYSINWVRQAPGKGLEWVGWINTETREPAYAYD
FRGRFTFSADTSKSMAYLQINSLRAEDTAVYYCALDYTYGMDYWGQGTLVTVSSFVPVFLPAKPTTTPAPR
PPTPAPTIASQPLSLRPEACRPAAGGAVHTRGLDFACDIYIWAPLAGTCGVLLLSLVITLYCNHRNRVKFS
RSADAPAYQQGQNQLYNELNLGRREEYDVLDKRRGRDPEMGGKPQRRKNPQEGLYNELQKDKMAEAYS
EIGMKGERRRGKGHDGLYQGLSTATKDTYDALHMQALPPR
(AA, Polypeptide Anti-BCMA CAR containing K-R CD3z only)
SEQ ID NO: 2
MALPVTALLLPLALLLHAARPDIVLTQSPASLAVSPGQRATITCRASESVTILGSHLIHWYQQKPGQPPKL
LINLASNVNTGVPARFSGSGSGTDFTLTISSVEPEDTANYYCLQSRTLPRTFGQGTKVEIKGSTSGSGKPG
SGEGSTKGQIQLVQSGPELKKPGGSVKISCKASGYTFTDYSINWVRQAPGKGLEWVGWINTETREPAYAYD
FRGRFTFSADTSKSMAYLQINSLRAEDTAVYYCALDYTYGMDYWGQGTLVTVSSFVPVFLPAKPTTTPAPR
PPTPAPTIASQPLSLRPEACRPAAGGAVHTRGLDFACDIYIWAPLAGTCGVLLLSLVITLYCNHRNRVRFS
RSADAPAYQQGQNQLYNELNLGRREEYDVLDRRRGRDPEMGGRPQRRRNPQEGLYNELQRDRMAEAYS
EIGMRGERRRGRGHDGLYQGLSTATRDTYDALHMQALPPR
(AA, Polypeptide Anti-BCMA CAR containing K-A CD3z only)
SEQ ID NO: 3
MALPVTALLLPLALLLHAARPDIVLTQSPASLAVSPGQRATITCRASESVTILGSHLIHWYQQKPGQPPKL
LINLASNVNTGVPARFSGSGSGTDFTLTISSVEPEDTANYYCLQSRTLPRTFGQGTKVEIKGSTSGSGKPG
SGEGSTKGQIQLVQSGPELKKPGGSVKISCKASGYTFTDYSINWVRQAPGKGLEWVGWINTETREPAYAYD
FRGRFTFSADTSKSMAYLQINSLRAEDTAVYYCALDYTYGMDYWGQGTLVTVSSFVPVFLPAKPTTTPAPR
PPTPAPTIASQPLSLRPEACRPAAGGAVHTRGLDFACDIYIWAPLAGTCGVLLLSLVITLYCNHRNRVAFS
RSADAPAYQQGQNQLYNELNLGRREEYDVLDARRGRDPEMGGAPQRRANPQEGLYNELQADAMAEAYS
EIGMAGERRRGAGHDGLYQGLSTATADTYDALHMQALPPR
(AA, Polypeptide Anti-BCMA CAR containing K-D CD3z only)
SEQ ID NO: 4
MALPVTALLLPLALLLHAARPDIVLTQSPASLAVSPGQRATITCRASESVTILGSHLIHWYQQKPGQPPKL
LINLASNVNTGVPARFSGSGSGTDFTLTISSVEPEDTANYYCLQSRTLPRTFGQGTKVEIKGSTSGSGKPG
SGEGSTKGQIQLVQSGPELKKPGGSVKISCKASGYTFTDYSINWVRQAPGKGLEWVGWINTETREPAYAYD
FRGRFTFSADTSKSMAYLQINSLRAEDTAVYYCALDYTYGMDYWGQGTLVTVSSFVPVFLPAKPTTTPAPR
PPTPAPTIASQPLSLRPEACRPAAGGAVHTRGLDFACDIYIWAPLAGTCGVLLLSLVITLYCNHRNRVDES
RSADAPAYQQGQNQLYNELNLGRREEYDVLDDRRGRDPEMGGDPQRRDNPQEGLYNELQDDDMAEAYS
EIGMDGERRRGDGHDGLYQGLSTATDDTYDALHMQALPPR-
(AA, Polypeptide Anti-BCMA CAR containing K-E CD3z only)
SEQ ID NO: 5
MALPVTALLLPLALLLHAARPDIVLTQSPASLAVSPGQRATITCRASESVTILGSHLIHWYQQKPGQPPKL
LINLASNVNTGVPARFSGSGSGTDFTLTISSVEPEDTANYYCLQSRTLPRTFGQGTKVEIKGSTSGSGKPG
SGEGSTKGQIQLVQSGPELKKPGGSVKISCKASGYTFTDYSINWVRQAPGKGLEWVGWINTETREPAYAYD
FRGRFTFSADTSKSMAYLQINSLRAEDTAVYYCALDYTYGMDYWGQGTLVTVSSFVPVFLPAKPTTTPAPR
PPTPAPTIASQPLSLRPEACRPAAGGAVHTRGLDFACDIYIWAPLAGTCGVLLLSLVITLYCNHRNRVEFS
RSADAPAYQQGQNQLYNELNLGRREEYDVLDERRGRDPEMGGEPQRRENPQEGLYNELQEDEMAEAYS
EIGMEGERRRGEGHDGLYQGLSTATEDTYDALHMQALPPR
(nt, QRF+STQP Anti-BCMA CAR containing WT CD3z only)
SEQ ID NO: 6
atggcactgccagtcacagcactgctgctgccactggcactgctgctccacgccgccagacccgacatcgt
gctgacacagagcccagcaagcctggccgtgagcccaggccagagagccacaatcacatgcagagccagcg
agtccgtgacaatcctgggctcccacctgatccactggtaccagcagaagcccggacagccacccaaactg
ctgatcaacctggccagcaacgtgaacacaggcgtgccagccagattcagcggcagcggcagcggaaccga
cttcaccctgacaatcagctcagtggaaccagaagacaccgccaactactactgcctgcagagcagaacac
tgcccagaaccttcggccaaggcaccaaggtcgaaatcaagggctccacaagcggcagcggaaagcccgga
agcggcgagggaagcaccaagggccagatccagctggtgcagagcggccccgaactgaagaaacccggcgg
aagcgtgaaaatctcctgcaaggccagcggctacaccttcaccgactacagcatcaactgggtgcgccagg
ccccaggcaagggcctggaatgggtcggctggatcaacaccgagacaagagagcccgcctacgcctacgac
ttcagaggcagattcaccttcagcgcagacacaagcaagagcatggcctacctgcagatcaactccctgag
agcagaggacaccgccgtctactactgcgccctggactacacctacggcatggactactggggccagggca
ccctcgtgaccgtgtccagcttcgtgcccgtgttcctgccagccaagccaaccacaacaccagcacccaga
ccaccaacaccagcaccaacaatcgccagccagccactgtccctgaggccagaggcatgcagaccagcagc
aggcggagccgtgcacaccagaggactggacttcgcctgcgacatctacatctgggcaccactggccggaa
catgcggcgtgctgctgctgagcctggtcatcaccctgtactgcaaccaccggaacagagtgaagttcagc
agatccgccgacgcaccagcctaccagcagggacagaaccagctgtacaacgagctgaacctggggagaag
agaagagtacgacgtgctggataagcggagaggcagagaccctgagatgggcggcaagccccaaagacgga
agaacccacaagagggcctgtacaacgagctgcagaaagacaagatggccgaggcctacagcgagatcgga
atgaagggcgagcgcagaagaggcaagggacacgacggactgtaccagggcctgagcacagccaccaagga
tacctacgatgccctgcacatgcaggccctgccaccaagatga
(nt, QRF+STQP Anti-BCMA CAR containing K-R CD3z only)
SEQ ID NO: 7
atggcactgccagtcacagcactgctgctgccactggcactgctgctccacgccgccagacccgacatcgt
gctgacacagagcccagcaagcctggccgtgagcccaggccagagagccacaatcacatgcagagccagcg
agtccgtgacaatcctgggctcccacctgatccactggtaccagcagaagcccggacagccacccaaactg
ctgatcaacctggccagcaacgtgaacacaggcgtgccagccagattcagcggcagcggcagcggaaccga
cttcaccctgacaatcagctcagtggaaccagaagacaccgccaactactactgcctgcagagcagaacac
tgcccagaaccttcggccaaggcaccaaggtcgaaatcaagggctccacaagcggcagcggaaagcccgga
agcggcgagggaagcaccaagggccagatccagctggtgcagagcggccccgaactgaagaaacccggcgg
aagcgtgaaaatctcctgcaaggccagcggctacaccttcaccgactacagcatcaactgggtgcgccagg
ccccaggcaagggcctggaatgggtcggctggatcaacaccgagacaagagagcccgcctacgcctacgac
ttcagaggcagattcaccttcagcgcagacacaagcaagagcatggcctacctgcagatcaactccctgag
agcagaggacaccgccgtctactactgcgccctggactacacctacggcatggactactggggccagggca
ccctcgtgaccgtgtccagcttcgtgcccgtgttcctgccagccaagccaaccacaacaccagcacccaga
ccaccaacaccagcaccaacaatcgccagccagccactgtccctgaggccagaggcatgcagaccagcagc
aggcggagccgtgcacaccagaggactggacttcgcctgcgacatctacatctgggcaccactggccggaa
catgcggcgtgctgctgctgagcctggtcatcaccctgtactgcaaccaccggaaccgcgtcaggtttagt
agatccgctgacgcaccagcttatcagcagggccagaaccaactttataatgagctcaatcttggcaggag
ggaagagtacgatgtccttgaccgaaggcgaggtcgggacccagaaatgggcgggaggcctcaacgacgac
gcaatccacaagagggactttacaacgaactgcagcgggatagaatggcagaggcttacagcgagatagga
atgcgaggcgaaaggaggcgaggtagggggcacgatggtctctatcaaggcctgagtacggctacgagaga
cacatatgatgcgctgcatatgcaggccctccctcccaggtga
(nt, QRF+STQP Anti-BCMA CAR containing K-A CD3z only)
SEQ ID NO: 8
atggcactgccagtcacagcactgctgctgccactggcactgctgctccacgccgccagacccgacatcgt
gctgacacagagcccagcaagcctggccgtgagcccaggccagagagccacaatcacatgcagagccagcg
agtccgtgacaatcctgggctcccacctgatccactggtaccagcagaagcccggacagccacccaaactg
ctgatcaacctggccagcaacgtgaacacaggcgtgccagccagattcagcggcagcggcagcggaaccga
cttcaccctgacaatcagctcagtggaaccagaagacaccgccaactactactgcctgcagagcagaacac
tgcccagaaccttcggccaaggcaccaaggtcgaaatcaagggctccacaagcggcagcggaaagcccgga
agcggcgagggaagcaccaagggccagatccagctggtgcagagcggccccgaactgaagaaacccggcgg
aagcgtgaaaatctcctgcaaggccagcggctacaccttcaccgactacagcatcaactgggtgcgccagg
ccccaggcaagggcctggaatgggtcggctggatcaacaccgagacaagagagcccgcctacgcctacgac
ttcagaggcagattcaccttcagcgcagacacaagcaagagcatggcctacctgcagatcaactccctgag
agcagaggacaccgccgtctactactgcgccctggactacacctacggcatggactactggggccagggca
ccctcgtgaccgtgtccagcttcgtgcccgtgttcctgccagccaagccaaccacaacaccagcacccaga
ccaccaacaccagcaccaacaatcgccagccagccactgtccctgaggccagaggcatgcagaccagcagc
aggcggagccgtgcacaccagaggactggacttcgcctgcgacatctacatctgggcaccactggccggaa
catgcggcgtgctgctgctgagcctggtcatcaccctgtactgcaaccaccggaacagagtcgcattctcc
cggtccgctgacgcccctgcgtatcagcagggtcagaaccaactgtataatgagcttaatcttggaagaag
agaggaatacgacgtattggatgcaaggcgagggcgcgacccggaaatgggcggagcgccccagaggcgag
caaatcctcaagagggactttataacgagctgcaggcggatgctatggcggaagcatacagtgaaatagga
atggccggcgaacggcgcaggggcgcggggcatgatggtctgtatcagggtctttctactgcaacggccga
cacgtatgacgccttgcatatgcaggcgctgccccccaggtga
(nt, QRF+STQP Anti-BCMA CAR containing K-D CD3z only)
SEQ ID NO: 9
atggcactgccagtcacagcactgctgctgccactggcactgctgctccacgccgccagacccgacatcgt
gctgacacagagcccagcaagcctggccgtgagcccaggccagagagccacaatcacatgcagagccagcg
agtccgtgacaatcctgggctcccacctgatccactggtaccagcagaagcccggacagccacccaaactg
ctgatcaacctggccagcaacgtgaacacaggcgtgccagccagattcagcggcagcggcagcggaaccga
cttcaccctgacaatcagctcagtggaaccagaagacaccgccaactactactgcctgcagagcagaacac
tgcccagaaccttcggccaaggcaccaaggtcgaaatcaagggctccacaagcggcagcggaaagcccgga
agcggcgagggaagcaccaagggccagatccagctggtgcagagcggccccgaactgaagaaacccggcgg
aagcgtgaaaatctcctgcaaggccagcggctacaccttcaccgactacagcatcaactgggtgcgccagg
ccccaggcaagggcctggaatgggtcggctggatcaacaccgagacaagagagcccgcctacgcctacgac
ttcagaggcagattcaccttcagcgcagacacaagcaagagcatggcctacctgcagatcaactccctgag
agcagaggacaccgccgtctactactgcgccctggactacacctacggcatggactactggggccagggca
ccctcgtgaccgtgtccagcttcgtgcccgtgttcctgccagccaagccaaccacaacaccagcacccaga
ccaccaacaccagcaccaacaatcgccagccagccactgtccctgaggccagaggcatgcagaccagcagc
aggcggagccgtgcacaccagaggactggacttcgcctgcgacatctacatctgggcaccactggccggaa
catgcggcgtgctgctgctgagcctggtcatcaccctgtactgcaaccaccggaacagagtcgacttctcc
cggtccgctgacgcccctgcgtatcagcagggtcagaaccaactgtataatgagcttaatcttggaagaag
agaggaatacgacgtattggatgacaggcgagggcgcgacccggaaatgggcggagatccccagaggcgag
acaatcctcaagagggactttataacgagctgcaggatgatgacatggcggaagcatacagtgaaatagga
atggatggcgaacggcgcaggggcgacgggcatgatggtctgtatcagggtctttctactgcaacggacga
cacgtatgacgccttgcatatgcaggcgctgccccccaggtga
(nt, QRF+STQP Anti-BCMA CAR containing K-E CD3z only)
SEQ ID NO: 10
atggcactgccagtcacagcactgctgctgccactggcactgctgctccacgccgccagacccgacatcgt
gctgacacagagcccagcaagcctggccgtgagcccaggccagagagccacaatcacatgcagagccagcg
agtccgtgacaatcctgggctcccacctgatccactggtaccagcagaagcccggacagccacccaaactg
ctgatcaacctggccagcaacgtgaacacaggcgtgccagccagattcagcggcagcggcagcggaaccga
cttcaccctgacaatcagctcagtggaaccagaagacaccgccaactactactgcctgcagagcagaacac
tgcccagaaccttcggccaaggcaccaaggtcgaaatcaagggctccacaagcggcagcggaaagcccgga
agcggcgagggaagcaccaagggccagatccagctggtgcagagcggccccgaactgaagaaacccggcgg
aagcgtgaaaatctcctgcaaggccagcggctacaccttcaccgactacagcatcaactgggtgcgccagg
ccccaggcaagggcctggaatgggtcggctggatcaacaccgagacaagagagcccgcctacgcctacgac
ttcagaggcagattcaccttcagcgcagacacaagcaagagcatggcctacctgcagatcaactccctgag
agcagaggacaccgccgtctactactgcgccctggactacacctacggcatggactactggggccagggca
ccctcgtgaccgtgtccagcttcgtgcccgtgttcctgccagccaagccaaccacaacaccagcacccaga
ccaccaacaccagcaccaacaatcgccagccagccactgtccctgaggccagaggcatgcagaccagcagc
aggcggagccgtgcacaccagaggactggacttcgcctgcgacatctacatctgggcaccactggccggaa
catgcggcgtgctgctgctgagcctggtcatcaccctgtactgcaaccaccggaacagagtcgaattctcc
cggtccgctgacgcccctgcgtatcagcagggtcagaaccaactgtataatgagcttaatcttggaagaag
agaggaatacgacgtattggatgagaggcgagggcgcgacccggaaatgggcggagaaccccagaggcgag
agaatcctcaagagggactttataacgagctgcaggaagatgagatggcggaagcatacagtgaaatagga
atggaaggcgaacggcgcaggggcgaagggcatgatggtctgtatcagggtctttctactgcaacggaaga
cacgtatgacgccttgcatatgcaggcgctgccccccaggtga
(nt, 5′UTR)
SEQ ID NO: 11
aggactcttctggtccccacagactcagagagaacccaccgccacc
(nt, 3′UTR)
SEQ ID NO: 12
tgcccgtcctcaccaagactgactgcctgctgctttgctactgcccgggcccatgagactgacttcccact
gctctgcctgcctctccccactgcactggcacagccccgccttgccgctgctgatccattgccggtgtgac
ccaagcacgcagcaatgcagctcaaaacgcttagcctagccacacccccacgggaaacagcagtgattaac
ctttagcaataaacgaaagtttaactaagctatactaaccccagggttggtcaatttcgtgccagccacac
ca
(nt, IVT mRNA)
SEQ ID NO: 13
aggactcttctggtccccacagactcagagagaacccaccgccaccatggcactgccagtcacagcactgc
tgctgccactggcactgctgctccacgccgccagacccgacatcgtgctgacacagagcccagcaagcctg
gccgtgagcccaggccagagagccacaatcacatgcagagccagcgagtccgtgacaatcctgggctccca
cctgatccactggtaccagcagaagcccggacagccacccaaactgctgatcaacctggccagcaacgtga
acacaggcgtgccagccagattcagcggcagcggcagcggaaccgacttcaccctgacaatcagctcagtg
gaaccagaagacaccgccaactactactgcctgcagagcagaacactgcccagaaccttcggccaaggcac
caaggtcgaaatcaagggctccacaagcggcagcggaaagcccggaagcggcgagggaagcaccaagggcc
agatccagctggtgcagagcggccccgaactgaagaaacccggcggaagcgtgaaaatctcctgcaaggcc
agcggctacaccttcaccgactacagcatcaactgggtgcgccaggccccaggcaagggcctggaatgggt
cggctggatcaacaccgagacaagagagcccgcctacgcctacgacttcagaggcagattcaccttcagcg
cagacacaagcaagagcatggcctacctgcagatcaactccctgagagcagaggacaccgccgtctactac
tgcgccctggactacacctacggcatggactactggggccagggcaccctcgtgaccgtgtccagcttcgt
gcccgtgttcctgccagccaagccaaccacaacaccagcacccagaccaccaacaccagcaccaacaatcg
ccagccagccactgtccctgaggccagaggcatgcagaccagcagcaggcggagccgtgcacaccagagga
ctggacttcgcctgcgacatctacatctgggcaccactggccggaacatgcggcgtgctgctgctgagcct
ggtcatcaccctgtactgcaaccaccggaacagagtgaagttcagcagatccgccgacgcaccagcctacc
agcagggacagaaccagctgtacaacgagctgaacctggggagaagagaagagtacgacgtgctggataag
cggagaggcagagaccctgagatgggcggcaagccccaaagacggaagaacccacaagagggcctgtacaa
cgagctgcagaaagacaagatggccgaggcctacagcgagatcggaatgaagggcgagcgcagaagaggca
agggacacgacggactgtaccagggcctgagcacagccaccaaggatacctacgatgccctgcacatgcag
gccctgccaccaagatgatgcccgtcctcaccaagactgactgcctgctgctttgctactgcccgggccca
tgagactgacttcccactgctctgcctgcctctccccactgcactggcacagccccgccttgccgctgctg
atccattgccggtgtgacccaagcacgcagcaatgcagctcaaaacgcttagcctagccacacccccacgg
gaaacagcagtgattaacctttagcaataaacgaaagtttaactaagctatactaaccccagggttggtca
atttcgtgccagccacacca
(nt, IVT mRNA)
SEQ ID NO: 14
aggactcttctggtccccacagactcagagagaacccaccgccaccatggcactgccagtcacagcactgc
tgctgccactggcactgctgctccacgccgccagacccgacatcgtgctgacacagagcccagcaagcctg
gccgtgagcccaggccagagagccacaatcacatgcagagccagcgagtccgtgacaatcctgggctccca
cctgatccactggtaccagcagaagcccggacagccacccaaactgctgatcaacctggccagcaacgtga
acacaggcgtgccagccagattcagcggcagcggcagcggaaccgacttcaccctgacaatcagctcagtg
gaaccagaagacaccgccaactactactgcctgcagagcagaacactgcccagaaccttcggccaaggcac
caaggtcgaaatcaagggctccacaagcggcagcggaaagcccggaagcggcgagggaagcaccaagggcc
agatccagctggtgcagagcggccccgaactgaagaaacccggcggaagcgtgaaaatctcctgcaaggcc
agcggctacaccttcaccgactacagcatcaactgggtgcgccaggccccaggcaagggcctggaatgggt
cggctggatcaacaccgagacaagagagcccgcctacgcctacgacttcagaggcagattcaccttcagcg
cagacacaagcaagagcatggcctacctgcagatcaactccctgagagcagaggacaccgccgtctactac
tgcgccctggactacacctacggcatggactactggggccagggcaccctcgtgaccgtgtccagcttcgt
gcccgtgttcctgccagccaagccaaccacaacaccagcacccagaccaccaacaccagcaccaacaatcg
ccagccagccactgtccctgaggccagaggcatgcagaccagcagcaggcggagccgtgcacaccagagga
ctggacttcgcctgcgacatctacatctgggcaccactggccggaacatgcggcgtgctgctgctgagcct
ggtcatcaccctgtactgcaaccaccggaaccgcgtcaggtttagtagatccgctgacgcaccagcttatc
agcagggccagaaccaactttataatgagctcaatcttggcaggagggaagagtacgatgtccttgaccga
aggcgaggtcgggacccagaaatgggcgggaggcctcaacgacgacgcaatccacaagagggactttacaa
cgaactgcagcgggatagaatggcagaggcttacagcgagataggaatgcgaggcgaaaggaggcgaggta
gggggcacgatggtctctatcaaggcctgagtacggctacgagagacacatatgatgcgctgcatatgcag
gccctccctcccaggtgatgcccgtcctcaccaagactgactgcctgctgctttgctactgcccgggccca
tgagactgacttcccactgctctgcctgcctctccccactgcactggcacagccccgccttgccgctgctg
atccattgccggtgtgacccaagcacgcagcaatgcagctcaaaacgcttagcctagccacacccccacgg
gaaacagcagtgattaacctttagcaataaacgaaagtttaactaagctatactaaccccagggttggtca
atttcgtgccagccacacca
(nt, IVT mRNA)
SEQ ID NO: 15
aggactcttctggtccccacagactcagagagaacccaccgccaccatggcactgccagtcacagcactgc
tgctgccactggcactgctgctccacgccgccagacccgacatcgtgctgacacagagcccagcaagcctg
gccgtgagcccaggccagagagccacaatcacatgcagagccagcgagtccgtgacaatcctgggctccca
cctgatccactggtaccagcagaagcccggacagccacccaaactgctgatcaacctggccagcaacgtga
acacaggcgtgccagccagattcagcggcagcggcagcggaaccgacttcaccctgacaatcagctcagtg
gaaccagaagacaccgccaactactactgcctgcagagcagaacactgcccagaaccttcggccaaggcac
caaggtcgaaatcaagggctccacaagcggcagcggaaagcccggaagcggcgagggaagcaccaagggcc
agatccagctggtgcagagcggccccgaactgaagaaacccggcggaagcgtgaaaatctcctgcaaggcc
agcggctacaccttcaccgactacagcatcaactgggtgcgccaggccccaggcaagggcctggaatgggt
cggctggatcaacaccgagacaagagagcccgcctacgcctacgacttcagaggcagattcaccttcagcg
cagacacaagcaagagcatggcctacctgcagatcaactccctgagagcagaggacaccgccgtctactac
tgcgccctggactacacctacggcatggactactggggccagggcaccctcgtgaccgtgtccagcttcgt
gcccgtgttcctgccagccaagccaaccacaacaccagcacccagaccaccaacaccagcaccaacaatcg
ccagccagccactgtccctgaggccagaggcatgcagaccagcagcaggcggagccgtgcacaccagagga
ctggacttcgcctgcgacatctacatctgggcaccactggccggaacatgcggcgtgctgctgctgagcct
ggtcatcaccctgtactgcaaccaccggaacagagtcgcattctcccggtccgctgacgcccctgcgtatc
agcagggtcagaaccaactgtataatgagcttaatcttggaagaagagaggaatacgacgtattggatgca
aggcgagggcgcgacccggaaatgggcggagcgccccagaggcgagcaaatcctcaagagggactttataa
cgagctgcaggcggatgctatggcggaagcatacagtgaaataggaatggccggcgaacggcgcaggggcg
cggggcatgatggtctgtatcagggtctttctactgcaacggccgacacgtatgacgccttgcatatgcag
gcgctgccccccaggtgatgcccgtcctcaccaagactgactgcctgctgctttgctactgcccgggccca
tgagactgacttcccactgctctgcctgcctctccccactgcactggcacagccccgccttgccgctgctg
atccattgccggtgtgacccaagcacgcagcaatgcagctcaaaacgcttagcctagccacacccccacgg
gaaacagcagtgattaacctttagcaataaacgaaagtttaactaagctatactaaccccagggttggtca
atttcgtgccagccacacca
(nt, IVT mRNA)
SEQ ID NO: 16
aggactcttctggtccccacagactcagagagaacccaccgccaccatggcactgccagtcacagcactgc
tgctgccactggcactgctgctccacgccgccagacccgacatcgtgctgacacagagcccagcaagcctg
gccgtgagcccaggccagagagccacaatcacatgcagagccagcgagtccgtgacaatcctgggctccca
cctgatccactggtaccagcagaagcccggacagccacccaaactgctgatcaacctggccagcaacgtga
acacaggcgtgccagccagattcagcggcagcggcagcggaaccgacttcaccctgacaatcagctcagtg
gaaccagaagacaccgccaactactactgcctgcagagcagaacactgcccagaaccttcggccaaggcac
caaggtcgaaatcaagggctccacaagcggcagcggaaagcccggaagcggcgagggaagcaccaagggcc
agatccagctggtgcagagcggccccgaactgaagaaacccggcggaagcgtgaaaatctcctgcaaggcc
agcggctacaccttcaccgactacagcatcaactgggtgcgccaggccccaggcaagggcctggaatgggt
cggctggatcaacaccgagacaagagagcccgcctacgcctacgacttcagaggcagattcaccttcagcg
cagacacaagcaagagcatggcctacctgcagatcaactccctgagagcagaggacaccgccgtctactac
tgcgccctggactacacctacggcatggactactggggccagggcaccctcgtgaccgtgtccagcttcgt
gcccgtgttcctgccagccaagccaaccacaacaccagcacccagaccaccaacaccagcaccaacaatcg
ccagccagccactgtccctgaggccagaggcatgcagaccagcagcaggcggagccgtgcacaccagagga
ctggacttcgcctgcgacatctacatctgggcaccactggccggaacatgcggcgtgctgctgctgagcct
ggtcatcaccctgtactgcaaccaccggaacagagtcgacttctcccggtccgctgacgcccctgcgtatc
agcagggtcagaaccaactgtataatgagcttaatcttggaagaagagaggaatacgacgtattggatgac
aggcgagggcgcgacccggaaatgggcggagatccccagaggcgagacaatcctcaagagggactttataa
cgagctgcaggatgatgacatggcggaagcatacagtgaaataggaatggatggcgaacggcgcaggggcg
acgggcatgatggtctgtatcagggtctttctactgcaacggacgacacgtatgacgccttgcatatgcag
gcgctgccccccaggtgatgcccgtcctcaccaagactgactgcctgctgctttgctactgcccgggccca
tgagactgacttcccactgctctgcctgcctctccccactgcactggcacagccccgccttgccgctgctg
atccattgccggtgtgacccaagcacgcagcaatgcagctcaaaacgcttagcctagccacacccccacgg
gaaacagcagtgattaacctttagcaataaacgaaagtttaactaagctatactaaccccagggttggtca
atttcgtgccagccacacca
(nt, IVT mRNA)
SEQ ID NO: 17
aggactcttctggtccccacagactcagagagaacccaccgccaccatggcactgccagtcacagcactgc
tgctgccactggcactgctgctccacgccgccagacccgacatcgtgctgacacagagcccagcaagcctg
gccgtgagcccaggccagagagccacaatcacatgcagagccagcgagtccgtgacaatcctgggctccca
cctgatccactggtaccagcagaagcccggacagccacccaaactgctgatcaacctggccagcaacgtga
acacaggcgtgccagccagattcagcggcagcggcagcggaaccgacttcaccctgacaatcagctcagtg
gaaccagaagacaccgccaactactactgcctgcagagcagaacactgcccagaaccttcggccaaggcac
caaggtcgaaatcaagggctccacaagcggcagcggaaagcccggaagcggcgagggaagcaccaagggcc
agatccagctggtgcagagcggccccgaactgaagaaacccggcggaagcgtgaaaatctcctgcaaggcc
agcggctacaccttcaccgactacagcatcaactgggtgcgccaggccccaggcaagggcctggaatgggt
cggctggatcaacaccgagacaagagagcccgcctacgcctacgacttcagaggcagattcaccttcagcg
cagacacaagcaagagcatggcctacctgcagatcaactccctgagagcagaggacaccgccgtctactac
tgcgccctggactacacctacggcatggactactggggccagggcaccctcgtgaccgtgtccagcttcgt
gcccgtgttcctgccagccaagccaaccacaacaccagcacccagaccaccaacaccagcaccaacaatcg
ccagccagccactgtccctgaggccagaggcatgcagaccagcagcaggcggagccgtgcacaccagagga
ctggacttcgcctgcgacatctacatctgggcaccactggccggaacatgcggcgtgctgctgctgagcct
ggtcatcaccctgtactgcaaccaccggaacagagtcgaattctcccggtccgctgacgcccctgcgtatc
agcagggtcagaaccaactgtataatgagcttaatcttggaagaagagaggaatacgacgtattggatgag
aggcgagggcgcgacccggaaatgggcggagaaccccagaggcgagagaatcctcaagagggactttataa
cgagctgcaggaagatgagatggcggaagcatacagtgaaataggaatggaaggcgaacggcgcaggggcg
aagggcatgatggtctgtatcagggtctttctactgcaacggaagacacgtatgacgccttgcatatgcag
gcgctgccccccaggtgatgcccgtcctcaccaagactgactgcctgctgctttgctactgcccgggccca
tgagactgacttcccactgctctgcctgcctctccccactgcactggcacagccccgccttgccgctgctg
atccattgccggtgtgacccaagcacgcagcaatgcagctcaaaacgcttagcctagccacacccccacgg
gaaacagcagtgattaacctttagcaataaacgaaagtttaactaagctatactaaccccagggttggtca
atttcgtgccagccacacca
(AA, Polypeptide WT CD3z intracellular domain)
SEQ ID NO: 18
RVKFSRSADAPAYQQGQNQLYNELNLGRREEYDVLDKRRGRDPEMGGKPQRRKNPQEGLYNELQKDKM
AEAYSEIGMKGERRRGKGHDGLYQGLSTATKDTYDALHMQALPPR
(AA, Polypeptide single K-A CD3z intracellular domain)
SEQ ID NO: 19
RVAFSRSADAPAYQQGQNQLYNELNLGRREEYDVLDKRRGRDPEMGGKPQRRKNPQEGLYNELQKDKM
AEAYSEIGMKGERRRGKGHDGLYQGLSTATKDTYDALHMQALPPR
(AA, Polypeptide single K-A CD3z intracellular domain)
SEQ ID NO: 20
RVKFSRSADAPAYQQGQNQLYNELNLGRREEYDVLDARRGRDPEMGGKPQRRKNPQEGLYNELQKDKM
AEAYSEIGMKGERRRGKGHDGLYQGLSTATKDTYDALHMQALPPR
(AA, Polypeptide single K-A CD3z intracellular domain)
SEQ ID NO: 21
RVKFSRSADAPAYQQGQNQLYNELNLGRREEYDVLDKRRGRDPEMGGAPQRRKNPQEGLYNELQKDKM
AEAYSEIGMKGERRRGKGHDGLYQGLSTATKDTYDALHMQALPPR
(AA, Polypeptide single K-A CD3z intracellular domain)
SEQ ID NO: 22
RVKFSRSADAPAYQQGQNQLYNELNLGRREEYDVLDKRRGRDPEMGGKPQRRANPQEGLYNELQKDKM
AEAYSEIGMKGERRRGKGHDGLYQGLSTATKDTYDALHMQALPPR
(AA, Polypeptide single K-A CD3z intracellular domain)
SEQ ID NO: 23
RVKFSRSADAPAYQQGQNQLYNELNLGRREEYDVLDKRRGRDPEMGGKPQRRKNPQEGLYNELQADKM
AEAYSEIGMKGERRRGKGHDGLYQGLSTATKDTYDALHMQALPPR
(AA, Polypeptide single K-A CD3z intracellular domain)
SEQ ID NO: 24
RVKFSRSADAPAYQQGQNQLYNELNLGRREEYDVLDKRRGRDPEMGGKPQRRKNPQEGLYNELQKDAM
AEAYSEIGMKGERRRGKGHDGLYQGLSTATKDTYDALHMQALPPR
(AA, Polypeptide single K-A CD3z intracellular domain)
SEQ ID NO: 25
RVKFSRSADAPAYQQGQNQLYNELNLGRREEYDVLDKRRGRDPEMGGKPQRRKNPQEGLYNELQKDKM
AEAYSEIGMAGERRRGKGHDGLYQGLSTATKDTYDALHMQALPPR
(AA, Polypeptide single K-A CD3z intracellular domain)
SEQ ID NO: 26
RVKFSRSADAPAYQQGQNQLYNELNLGRREEYDVLDKRRGRDPEMGGKPQRRKNPQEGLYNELQKDKM
AEAYSEIGMKGERRRGAGHDGLYQGLSTATKDTYDALHMQALPPR
(AA, Polypeptide single K-A CD3z intracellular domain)
SEQ ID NO: 27
RVKFSRSADAPAYQQGQNQLYNELNLGRREEYDVLDKRRGRDPEMGGKPQRRKNPQEGLYNELQKDKM
AEAYSEIGMKGERRRGKGHDGLYQGLSTATADTYDALHMQALPPR
(AA, Polypeptide 2-7 K-A CD3z intracellular domain)
SEQ ID NO: 28
RVKFSRSADAPAYQQGQNQLYNELNLGRREEYDVLDARRGRDPEMGGKPQRRANPQEGLYNELQADAM
AEAYSEIGMAGERRRGAGHDGLYQGLSTATADTYDALHMQALPPR
(AA, Polypeptide 2-7 K-A CD3z intracellular domain)
SEQ ID NO: 29
RVKFSRSADAPAYQQGQNQLYNELNLGRREEYDVLDARRGRDPEMGGKPQRRKNPQEGLYNELQADAM
AEAYSEIGMAGERRRGAGHDGLYQGLSTATADTYDALHMQALPPR
(AA, Polypeptide 2-7 K-A CD3z intracellular domain)
SEQ ID NO: 30
RVAFSRSADAPAYQQGQNQLYNELNLGRREEYDVLDKRRGRDPEMGGAPQRRKNPQEGLYNELQKDKM
AEAYSEIGMKGERRRGKGHDGLYQGLSTATKDTYDALHMQALPPR
(AA, Polypeptide 2-7 K-A CD3z intracellular domain)
SEQ ID NO: 31
RVAFSRSADAPAYQQGQNQLYNELNLGRREEYDVLDARRGRDPEMGGAPQRRANPQEGLYNELQKDKM
AEAYSEIGMAGERRRGAGHDGLYQGLSTATADTYDALHMQALPPR
(AA, Polypeptide 2-7 K-A CD3z intracellular domain)
SEQ ID NO: 32
RVAFSRSADAPAYQQGQNQLYNELNLGRREEYDVLDARRGRDPEMGGAPQRRANPQEGLYNELQADAM
AEAYSEIGMKGERRRGKGHDGLYQGLSTATADTYDALHMQALPPR
(AA, Polypeptide 2-7 K-A CD3z intracellular domain)
SEQ ID NO: 33
RVKFSRSADAPAYQQGQNQLYNELNLGRREEYDVLDARRGRDPEMGGAPQRRANPQEGLYNELQADAM
AEAYSEIGMAGERRRGAGHDGLYQGLSTATKDTYDALHMQALPPR
(AA, Polypeptide 8 K-A CD3z intracellular domain)
SEQ ID NO: 34
RVKFSRSADAPAYQQGQNQLYNELNLGRREEYDVLDARRGRDPEMGGAPQRRANPQEGLYNELQADAM
AEAYSEIGMAGERRRGAGHDGLYQGLSTATADTYDALHMQALPPR
(AA, Polypeptide 8 K-A CD3z intracellular domain)
SEQ ID NO: 35
RVAFSRSADAPAYQQGQNQLYNELNLGRREEYDVLDARRGRDPEMGGKPQRRANPQEGLYNELQADAM
AEAYSEIGMAGERRRGAGHDGLYQGLSTATADTYDALHMQALPPR
(AA, Polypeptide 8 K-A CD3z intracellular domain)
SEQ ID NO: 36
RVAFSRSADAPAYQQGQNQLYNELNLGRREEYDVLDARRGRDPEMGGAPQRRANPQEGLYNELQADKM
AEAYSEIGMAGERRRGAGHDGLYQGLSTATADTYDALHMQALPPR
(AA, Polypeptide 8 K-A CD3z intracellular domain)
SEQ ID NO: 37
RVAFSRSADAPAYQQGQNQLYNELNLGRREEYDVLDARRGRDPEMGGAPQRRANPQEGLYNELQADAM
AEAYSEIGMAGERRRGKGHDGLYQGLSTATADTYDALHMQALPPR
(AA, Polypeptide 8 K-A CD3z intracellular domain)
SEQ ID NO: 38
RVAFSRSADAPAYQQGQNQLYNELNLGRREEYDVLDKRRGRDPEMGGAPQRRANPQEGLYNELQADAM
AEAYSEIGMAGERRRGAGHDGLYQGLSTATADTYDALHMQALPPR
(AA, Polypeptide 8 K-A CD3z intracellular domain)
SEQ ID NO: 39
RVAFSRSADAPAYQQGQNQLYNELNLGRREEYDVLDARRGRDPEMGGAPQRRKNPQEGLYNELQADAM
AEAYSEIGMAGERRRGAGHDGLYQGLSTATADTYDALHMQALPPR
(AA, Polypeptide 8 K-A CD3z intracellular domain)
SEQ ID NO: 40
RVAFSRSADAPAYQQGQNQLYNELNLGRREEYDVLDARRGRDPEMGGAPQRRANPQEGLYNELQKDAM
AEAYSEIGMAGERRRGAGHDGLYQGLSTATADTYDALHMQALPPR
(AA, Polypeptide 8 K-A CD3z intracellular domain)
SEQ ID NO: 41
RVAFSRSADAPAYQQGQNQLYNELNLGRREEYDVLDARRGRDPEMGGAPQRRANPQEGLYNELQADAM
AEAYSEIGMKGERRRGAGHDGLYQGLSTATADTYDALHMQALPPR
(AA, Polypeptide 8 K-A CD3z intracellular domain)
SEQ ID NO: 42
RVAFSRSADAPAYQQGQNQLYNELNLGRREEYDVLDARRGRDPEMGGAPQRRANPQEGLYNELQADAM
AEAYSEIGMAGERRRGAGHDGLYQGLSTATKDTYDALHMQALPPR
(AA, Polypeptide 9 K-A CD3z intracellular domain)
SEQ ID NO: 43
RVAFSRSADAPAYQQGQNQLYNELNLGRREEYDVLDARRGRDPEMGGAPQRRANPQEGLYNELQADAM
AEAYSEIGMAGERRRGAGHDGLYQGLSTATADTYDALHMQALPPR
(AA, Polypeptide 9 K-V CD3z intracellular domain)
SEQ ID NO: 44
RVVFSRSADAPAYQQGQNQLYNELNLGRREEYDVLDVRRGRDPEMGGVPQRRVNPQEGLYNELQVDVM
AEAYSEIGMVGERRRGVGHDGLYQGLSTATVDTYDALHMQALPPR
(AA, Polypeptide 9 K-I CD3z intracellular domain)
SEQ ID NO: 45
RVIFSRSADAPAYQQGQNQLYNELNLGRREEYDVLDIRRGRDPEMGGIPQRRINPQEGLYNELQIDIM
AEAYSEIGMIGERRRGIGHDGLYQGLSTATIDTYDALHMQALPPR
(AA, Polypeptide 9 K-L CD3z intracellular domain)
SEQ ID NO: 46
RVLFSRSADAPAYQQGQNQLYNELNLGRREEYDVLDLRRGRDPEMGGLPQRRLNPQEGLYNELQLDLM
AEAYSEIGMLGERRRGLGHDGLYQGLSTATLDTYDALHMQALPPR
(AA, Polypeptide 9 K-M CD3z intracellular domain)
SEQ ID NO: 47
RVMFSRSADAPAYQQGQNQLYNELNLGRREEYDVLDMRRGRDPEMGGMPQRRMNPQEGLYNELQMDMM
AEAYSEIGMMGERRRGMGHDGLYQGLSTATMDTYDALHMQALPPR
(AA, Polypeptide 9 K-F CD3z intracellular domain)
SEQ ID NO: 48
RVFFSRSADAPAYQQGQNQLYNELNLGRREEYDVLDFRRGRDPEMGGEPQRRENPQEGLYNELQEDEM
AEAYSEIGMFGERRRGFGHDGLYQGLSTATFDTYDALHMQALPPR
(AA, Polypeptide 9 K-Y CD3z intracellular domain)
SEQ ID NO: 49
RVYFSRSADAPAYQQGQNQLYNELNLGRREEYDVLDYRRGRDPEMGGYPQRRYNPQEGLYNELQYDYM
AEAYSEIGMYGERRRGYGHDGLYQGLSTATYDTYDALHMQALPPR
(AA, Polypeptide 9 K-S CD3z intracellular domain)
SEQ ID NO: 50
RVSFSRSADAPAYQQGQNQLYNELNLGRREEYDVLDSRRGRDPEMGGSPQRRSNPQEGLYNELQSDSM
AEAYSEIGMSGERRRGSGHDGLYQGLSTATSDTYDALHMQALPPR
(AA, Polypeptide 9 K-T CD3z intracellular domain)
SEQ ID NO: 51
RVTFSRSADAPAYQQGQNQLYNELNLGRREEYDVLDTRRGRDPEMGGTPQRRTNPQEGLYNELQTDTM
AEAYSEIGMTGERRRGTGHDGLYQGLSTATTDTYDALHMQALPPR
(AA, Polypeptide 9 K-N CD3z intracellular domain)
SEQ ID NO: 52
RVNFSRSADAPAYQQGQNQLYNELNLGRREEYDVLDNRRGRDPEMGGNPQRRNNPQEGLYNELQNDNM
AEAYSEIGMNGERRRGNGHDGLYQGLSTATNDTYDALHMQALPPR
(AA, Polypeptide 9 K-Q CD3z intracellular domain)
SEQ ID NO: 53
RVQFSRSADAPAYQQGQNQLYNELNLGRREEYDVLDQRRGRDPEMGGQPQRRQNPQEGLYNELQQDQM
AEAYSEIGMQGERRRGQGHDGLYQGLSTATQDTYDALHMQALPPR
(AA, Polypeptide 9 K-H CD3z intracellular domain)
SEQ ID NO: 54
RVHFSRSADAPAYQQGQNQLYNELNLGRREEYDVLDHRRGRDPEMGGHPQRRHNPQEGLYNELQHDHM
AEAYSEIGMHGERRRGHGHDGLYQGLSTATHDTYDALHMQALPPR
(AA, Polypeptide 9 K-D CD3z intracellular domain)
SEQ ID NO: 55
RVDFSRSADAPAYQQGQNQLYNELNLGRREEYDVLDDRRGRDPEMGGDPQRRDNPQEGLYNELQDDDM
AEAYSEIGMDGERRRGDGHDGLYQGLSTATDDTYDALHMQALPPR
(AA, Polypeptide 9 K-E CD3z intracellular domain)
SEQ ID NO: 56
RVEFSRSADAPAYQQGQNQLYNELNLGRREEYDVLDERRGRDPEMGGEPQRRENPQEGLYNELQEDEM
AEAYSEIGMEGERRRGEGHDGLYQGLSTATEDTYDALHMQALPPR
(AA, Polypeptide WT CD28 K-A CD3z intracellular domain)
SEQ ID NO: 57
RSKRSRLLHSDYMNMTPRRPGPTRKHYQPYAPPRDFAAYRSRVAFSRSADAPAYQQGQNQLYNELNLG
RREEYDVLDARRGRDP EMGGAPQRRANPQEGLYNELQADAMAEAYSEIGMAGERRRGAGHDGLYQGLS
TATADTYDALHMQALPPR
(AA, Polypeptide K-A CD28 K-A CD3z intracellular domain)
SEQ ID NO: 58
RSARSRLLHSDYMNMTPRRPGPTRAHYQPYAPPRDFAAYRSRVAFSRSADAPAYQQGQNQLYNELNLG
RREEYDVLDARRGRDP EMGGAPQRRANPQEGLYNELQADAMAEAYSEIGMAGERRRGAGHDGLYQGLS
TATADTYDALHMQALPPR
(AA, Polypeptide WT 41BB K-A CD3z intracellular domain)
SEQ ID NO: 59
KRGRKKLLYIFKQPFMRPVQTTQEEDGCSCRFPEEEEGGCELRVAFSRSADAPAYQQGQNQLYNELNL
GRREEYDVLDARRGRDPEMGGAPQRRANPQEGLYNELQADAMAEAYSEIGMAGERRRGAGHDGLYQGL
STATADTYDALHMQALPPR
(AA, Polypeptide K-A 41BB K-A CD3z intracellular domain)
SEQ ID NO: 60
ARGRAALLYIFAQPFMRPVQTTQEEDGCSCRFPEEEEGGCELRVAFSRSADAPAYQQGQNQLYNELNL
GRREEYDVLDARRGRDPEMGGAPQRRANPQEGLYNELQADAMAEAYSEIGMAGERRRGAGHDGLYQGL
STATADTYDALHMQALPPR
(AA, Polypeptide WT CD3z intracellular domain
(polymorphic variant)
SEQ ID NO: 61
RVKFSRSADAPAYQQGQNQLYNELNLGRREEYDVLDKRRGRDPEMGGKPRRKNPQEGLYNELQKDKMA
EAYSEIGMKGERRRGKGHDGLYQGLSTATKDTYDALHMQALPPR
(AA, Polypeptide K-E CD28 K-A CD3z intracellular domain)
SEQ ID NO: 62
RSERSRLLHSDYMNMTPRRPGPTREHYQPYAPPRDFAAYRSRVAFSRSADAPAYQQGQNQLYNELNLG
RREEYDVLDARRGRDP EMGGAPQRRANPQEGLYNELQADAMAEAYSEIGMAGERRRGAGHDGLYQGLS
TATADTYDALHMQALPPR
(AA, Polypeptide WT CD28 K-E CD3z intracellular domain)
SEQ ID NO: 63
RSKRSRLLHSDYMNMTPRRPGPTRKHYQPYAPPRDFAAYRSRVEFSRSADAPAYQQGQNQLYNELNLG
RREEYDVLDERRGRDP EMGGEPQRRENPQEGLYNELQEDEMAEAYSEIGMEGERRRGEGHDGLYQGLS
TATEDTYDALHMQALPPR
(AA, Polypeptide K-A CD28 K-E CD3z intracellular domain)
SEQ ID NO: 64
RSARSRLLHSDYMNMTPRRPGPTRAHYQPYAPPRDFAAYRSRVEFSRSADAPAYQQGQNQLYNELNLG
RREEYDVLDERRGRDPEMGGEPQRRENPQEGLYNELQEDEMAEAYSEIGMEGERRRGEGHDGLYQGLS
TATEDTYDALHMQALPPR
(AA, Polypeptide K-E CD28 K-E CD3z intracellular domain)
SEQ ID NO: 65
RSERSRLLHSDYMNMTPRRPGPTREHYQPYAPPRDFAAYRSRVEFSRSADAPAYQQGQNQLYNELNLG
RREEYDVLDERRGRDPEMGGEPQRRENPQEGLYNELQEDEMAEAYSEIGMEGERRRGEGHDGLYQGLS
TATEDTYDALHMQALPPR
(AA, Polypeptide WT CD28-CD3z intracellular domain
(polymorphic variant)
SEQ ID NO: 66
RSKRSRLLHSDYMNMTPRRPGPTRKHYQPYAPPRDFAAYRSRVKFSRSADAPAYQQGQNQLYNELNLG
RREEYDVLDKRRGRDPEMGGKPRRKNPQEGLYNELQKDKMAEAYSEIGMKGERRRGKGHDGLYQGLST
ATKDTYDALHMQALPPR
(nt, IVT mRNA)
SEQ ID NO: 67
AGGACTCTTCTGGTCCCCACAGACTCAGAGAGAACCCACCGCCACCATGGCACTGCCAGTCACAGCAC
TGCTGCTGCCACTGGCACTGCTGCTCCACGCCGCCAGACCCGACATCGTGCTGACACAGAGCCCAGCA
AGCCTGGCCGTGAGCCCAGGCCAGAGAGCCACAATCACATGCAGAGCCAGCGAGTCCGTGACAATCCT
GGGCTCCCACCTGATCCACTGGTACCAGCAGAAGCCCGGACAGCCACCCAAACTGCTGATCAACCTGG
CCAGCAACGTGAACACAGGCGTGCCAGCCAGATTCAGCGGCAGCGGCAGCGGAACCGACTTCACCCTG
ACAATCAGCTCAGTGGAACCAGAAGACACCGCCAACTACTACTGCCTGCAGAGCAGAACACTGCCCAG
AACCTTCGGCCAAGGCACCAAGGTCGAAATCAAGGGCTCCACAAGCGGCAGCGGAAAGCCCGGAAGCG
GCGAGGGAAGCACCAAGGGCCAGATCCAGCTGGTGCAGAGCGGCCCCGAACTGAAGAAACCCGGCGGA
AGCGTGAAAATCTCCTGCAAGGCCAGCGGCTACACCTTCACCGACTACAGCATCAACTGGGTGCGCCA
GGCCCCAGGCAAGGGCCTGGAATGGGTCGGCTGGATCAACACCGAGACAAGAGAGCCCGCCTACGCCT
ACGACTTCAGAGGCAGATTCACCTTCAGCGCAGACACAAGCAAGAGCATGGCCTACCTGCAGATCAAC
TCCCTGAGAGCAGAGGACACCGCCGTCTACTACTGCGCCCTGGACTACACCTACGGCATGGACTACTG
GGGCCAGGGCACCCTCGTGACCGTGTCCAGCTTCGTGCCCGTGTTCCTGCCAGCCAAGCCAACCACAA
CACCAGCACCCAGACCACCAACACCAGCACCAACAATCGCCAGCCAGCCACTGTCCCTGAGGCCAGAG
GCATGCAGACCAGCAGCAGGCGGAGCCGTGCACACCAGAGGACTGGACTTCGCCTGCGACATCTACAT
CTGGGCACCACTGGCCGGAACATGCGGCGTGCTGCTGCTGAGCCTGGTCATCACCCTGTACTGCAACC
ACCGGAACAGGTCCGAGAGATCTCGCCTTCTCCATTCTGACTACATGAACATGACGCCCCGCAGGCCA
GGGCCAACACGAGAGCATTATCAGCCATATGCCCCACCACGCGATTTTGCTGCGTACCGCTCTAGAGT
CGAATTCTCCCGGTCCGCTGACGCCCCTGCGTATCAGCAGGGTCAGAACCAACTGTATAATGAGCTTA
ATCTTGGAAGAAGAGAGGAATACGACGTATTGGATGAGAGGCGAGGGCGCGACCCGGAAATGGGCGGA
GAACCCCAGAGGCGAGAGAATCCTCAAGAGGGACTTTATAACGAGCTGCAGGAAGATGAGATGGCGGA
AGCATACAGTGAAATAGGAATGGAAGGCGAACGGCGCAGGGGCGAAGGGCATGATGGTCTGTATCAGG
GTCTTTCTACTGCAACGGAAGACACGTATGACGCCTTGCATATGCAGGCGCTGCCCCCCAGGTGATGC
CCGTCCTCACCAAGACTGACTGCCTGCTGCTTTGCTACTGCCCGGGCCCATGAGACTGACTTCCCACT
GCTCTGCCTGCCTCTCCCCACTGCACTGGCACAGCCCCGCCTTGCCGCTGCTGATCCATTGCCGGTGT
GACCCAAGCACGCAGCAATGCAGCTCAAAACGCTTAGCCTAGCCACACCCCCACGGGAAACAGCAGTG
ATTAACCTTTAGCAATAAACGAAAGTTTAACTAAGCTATACTAACCCCAGGGTTGGTCAATTTCGTGC
CAGCCACACCA
(AA, Polypeptide Anti-BCMA CAR containing K-E CD28-CD3z)
SEQ ID NO: 68
MALPVTALLLPLALLLHAARPDIVLTQSPASLAVSPGQRATITCRASESVTILGSHLI HWYQQKPGQP
PKLLINLASNVNTGVPARFSGSGSGTDFTLTISSVEPEDTANYYCLQSRTLPRTFGQGTKVEIKGSTS
GSGKPGSGEGSTKGQIQLVQSGPELKKPGGSVKISCKASGYTFTDYSINWVRQAPGKGLEWVGWINTE
TREPAYAYDFRGRFTF SADTSKSMAYLQINSLRAEDTAVYYCALDYTYGMDYWGQGTLVTVSSFVPVF
LPAKPTTTPAPRPPTPAPTIASQPLSLRPEACRPAAGGAVHTRGLDFACDIYIWAP LAGTCGVLLLSL
VITLYCNHRNRSERSRLLHSDYMNMTPRRPGPTREHYQPYAPPRDFAAYRSRVEFSRSADAPAYQQGQ
NQLYNELNLGRREEYDVLDERRGRDP EMGGEPQRRENPQEGLYNELQEDEMAEAYSEIGMEGERRRGE
GHDGLYQGLSTATEDTYDALHMQALPPR
(AA, Polypeptide CD3z intracellular domain K-E with one K-A
(pos 1))
SEQ ID NO: 69
RVAFSRSADAPAYQQGQNQLYNELNLGRREEYDVLDERRGRDPEMGGEPQRRENPQEGLYNELQEDEM
AEAYSEIGMEGERRRGEGHDGLYQGLSTATEDTYDALHMQALPPR
(AA, Polypeptide CD3z intracellular domain K-E with one K-A
(pos 2))
SEQ ID NO: 70
RVEFSRSADAPAYQQGQNQLYNELNLGRREEYDVLDARRGRDPEMGGEPQRRENPQEGLYNELQEDEM
AEAYSEIGMEGERRRGEGHDGLYQGLSTATEDTYDALHMQALPPR
(AA, Polypeptide CD3z intracellular domain K-E with one K-A
(pos 3))
SEQ ID NO: 71
RVEFSRSADAPAYQQGQNQLYNELNLGRREEYDVLDERRGRDPEMGGAPQRRENPQEGLYNELQEDEM
AEAYSEIGMEGERRRGEGHDGLYQGLSTATEDTYDALHMQALPPR
(AA, Polypeptide CD3z intracellular domain K-E with one K-A
(pos 4))
SEQ ID NO: 72
RVEFSRSADAPAYQQGQNQLYNELNLGRREEYDVLDERRGRDPEMGGEPQRRANPQEGLYNELQEDEM
AEAYSEIGMEGERRRGEGHDGLYQGLSTATEDTYDALHMQALPPR
(AA, Polypeptide CD3z intracellular domain K-E with one K-A
(pos 5))
SEQ ID NO: 73
RVEFSRSADAPAYQQGQNQLYNELNLGRREEYDVLDERRGRDPEMGGEPQRRENPQEGLYNELQADEM
AEAYSEIGMEGERRRGEGHDGLYQGLSTATEDTYDALHMQALPPR
(AA, Polypeptide CD3z intracellular domain K-E with one K-A
(pos 6))
SEQ ID NO: 74
RVEFSRSADAPAYQQGQNQLYNELNLGRREEYDVLDERRGRDPEMGGEPQRRENPQEGLYNELQEDAM
AEAYSEIGMEGERRRGEGHDGLYQGLSTATEDTYDALHMQALPPR
(AA, Polypeptide CD3z intracellular domain K-E with one K-A
(pos 7))
SEQ ID NO: 75
RVEFSRSADAPAYQQGQNQLYNELNLGRREEYDVLDERRGRDPEMGGEPQRRENPQEGLYNELQEDEM
AEAYSEIGMAGERRRGEGHDGLYQGLSTATEDTYDALHMQALPPR
(AA, Polypeptide CD3z intracellular domain K-E with one K-A
(pos 8))
SEQ ID NO: 76
RVEFSRSADAPAYQQGQNQLYNELNLGRREEYDVLDERRGRDPEMGGEPQRRENPQEGLYNELQEDEM
AEAYSEIGMEGERRRGAGHDGLYQGLSTATEDTYDALHMQALPPR
(AA, Polypeptide CD3z intracellular domain K-E with one K-A
(pos 9))
SEQ ID NO: 77
RVEFSRSADAPAYQQGQNQLYNELNLGRREEYDVLDERRGRDPEMGGEPQRRENPQEGLYNELQEDEM
AEAYSEIGMEGERRRGEGHDGLYQGLSTATADTYDALHMQALPPR
(AA, Polypeptide CD3z intracellular domain K-A with one K-E
(pos 1))
SEQ ID NO: 78
RVEFSRSADAPAYQQGQNQLYNELNLGRREEYDVLDARRGRDPEMGGAPQRRANPQEGLYNELQADAM
AEAYSEIGMAGERRRGAGHDGLYQGLSTATADTYDALHMQALPPR
(AA, Polypeptide CD3z intracellular domain K-A with one K-E
(pos 2))
SEQ ID NO: 79
RVAFSRSADAPAYQQGQNQLYNELNLGRREEYDVLDERRGRDPEMGGAPQRRANPQEGLYNELQADAM
AEAYSEIGMAGERRRGAGHDGLYQGLSTATADTYDALHMQALPPR
(AA, Polypeptide CD3z intracellular domain K-A with one K-E
(pos 3))
SEQ ID NO: 80
RVAFSRSADAPAYQQGQNQLYNELNLGRREEYDVLDARRGRDPEMGGEPQRRANPQEGLYNELQADAM
AEAYSEIGMAGERRRGAGHDGLYQGLSTATADTYDALHMQALPPR
(AA, Polypeptide CD3z intracellular domain K-A with one K-E
(pos 4))
SEQ ID NO: 81
RVAFSRSADAPAYQQGQNQLYNELNLGRREEYDVLDARRGRDPEMGGAPQRRENPQEGLYNELQADAM
AEAYSEIGMAGERRRGAGHDGLYQGLSTATADTYDALHMQALPPR
(AA, Polypeptide CD3z intracellular domain K-A with one K-E
(pos 5))
SEQ ID NO: 82
RVAFSRSADAPAYQQGQNQLYNELNLGRREEYDVLDARRGRDPEMGGAPQRRANPQEGLYNELQEDAM
AEAYSEIGMAGERRRGAGHDGLYQGLSTATADTYDALHMQALPPR
(AA, Polypeptide CD3z intracellular domain K-A with one K-E
(pos 6))
SEQ ID NO: 83
RVAFSRSADAPAYQQGQNQLYNELNLGRREEYDVLDARRGRDPEMGGAPQRRANPQEGLYNELQADEM
AEAYSEIGMAGERRRGAGHDGLYQGLSTATADTYDALHMQALPPR
(AA, Polypeptide CD3z intracellular domain K-A with one K-E
(pos 7))
SEQ ID NO: 84
RVAFSRSADAPAYQQGQNQLYNELNLGRREEYDVLDARRGRDPEMGGAPQRRANPQEGLYNELQADAM
AEAYSEIGMEGERRRGAGHDGLYQGLSTATADTYDALHMQALPPR
(AA, Polypeptide CD3z intracellular domain K-A with one K-E
(pos 8))
SEQ ID NO: 85
RVAFSRSADAPAYQQGQNQLYNELNLGRREEYDVLDARRGRDPEMGGAPQRRANPQEGLYNELQADAM
AEAYSEIGMAGERRRGEGHDGLYQGLSTATADTYDALHMQALPPR
(AA, Polypeptide CD3z intracellular domain K-A with one K-E
(pos 9))
SEQ ID NO: 86
RVAFSRSADAPAYQQGQNQLYNELNLGRREEYDVLDARRGRDPEMGGAPQRRANPQEGLYNELQADAM
AEAYSEIGMAGERRRGAGHDGLYQGLSTATEDTYDALHMQALPPR
(AA, scFv)
SEQ ID NO: 87
DIQMTQTTSSLSASLGDRVTISCRASQDISKYLNWYQQKPDGTVKLLIYHTSRLHSGVP SRFSGSGSGTDY
SLTISNLEQEDIATYFCQQGNTLPYTFGGGTKLEITGSTSGSGKPGSGEGSTKGEVKLQESGPGLVAPSQS
LSVTCTVSGVSLPDYGVSWIRQPPRKGLEWLGVIWGSETTYYNSALKSRLTIIKDNSKSQVFLKMNSLQTD
DTAIYYCAKHYYYGGSYAMDYWGQGTSVTVSS
(AA, scFv)
SEQ ID NO: 88
DIQMTQTTSSLSASLGDRVTISCRASQDISKYLNWYQQKPDGTVKLLIYHTSRLHSGVPSRFSGSGSGTDY
SLTISNLEQEDIATYFCQQGNTLPYTFGGGTKLEITGSTSGSGKPGSGEGSTKGEVKLQESGPGLVAPSQS
LSVTCTVSGVSLPDYGVSWIRQPPRKGLEWLGVIWGSETTYYNSALKSRLTIIKDNSKSQVFLKMNSLQTD
DTAIYYCAKHYYAGGSYAMDYWGQGTSVTVSS
(nt, Anti-BCMA CAR full length ntd)
SEQ ID NO: 89
aggactcttctggtccccacagactcagagagaacccaccgccaccatggcactgccagtcacagcactgc
tgctgccactggcactgctgctccacgccgccagacccgagcagaagctgatcagcgaggaggacctggac
atccagatgacacagactacatcctccctgtctgcctctctgggagacagagtcaccatcagttgcagggc
aagtcaggacattagtaaatatttaaattggtatcagcagaaaccagatggaactgttaaactcctgatct
accatacatcaagattacactcaggagtcccatcaaggttcagtggcagtgggtctggaacagattattct
ctcaccattagcaacctggagcaagaagatattgccacttacttttgccaacagggtaatacgcttccgta
cacgttcggaggggggaccaagctggagatcacaggctccacaagcggcagcggaaagcccggaagcggcg
agggaagcaccaagggcgaggtgaaactgcaggagtcaggacctggcctggtggcgccctcacagagcctg
tccgtcacatgcactgtctcaggggtctcattacccgactatggtgtaagctggattcgccagcctccacg
aaagggtctggagtggctgggagtaatatggggtagtgaaaccacatactataattcagctctcaaatcca
gactgaccatcatcaaggacaactccaagagccaagttttcttaaaaatgaacagtctgcaaactgatgac
acagccatttactactgtgccaaacattattactacggtggtagctatgctatggactactggggccaagg
aacctcagtcaccgtctcctcattcgtgcccgtgttcctgccagccaagccaaccacaacaccagcaccca
gaccaccaacaccagcaccaacaatcgccagccagccactgtccctgaggccagaggcatgcagaccagca
gcaggcggagccgtgcacaccagaggactggacttcgcctgcgacatctacatctgggcaccactggccgg
aacatgcggcgtgctgctgctgagcctggtcatcaccctgtactgcaaccaccggaacaggtccgagagat
ctcgccttctccattctgactacatgaacatgacgccccgcaggccagggccaacacgagagcattatcag
ccatatgccccaccacgcgattttgctgcgtaccgctctagagtcgaattctcccggtccgctgacgcccc
tgcgtatcagcagggtcagaaccaactgtataatgagcttaatcttggaagaagagaggaatacgacgtat
tggatgagaggcgagggcgcgacccggaaatgggcggagaaccccagaggcgagagaatcctcaagaggga
ctttataacgagctgcaggaagatgagatggcggaagcatacagtgaaataggaatggaaggcgaacggcg
caggggcgaagggcatgatggtctgtatcagggtctttctactgcaacggaagacacgtatgacgccttgc
atatgcaggcgctgccccccaggtgatgcccgtcctcaccaagactgactgcctgctgctttgctactgcc
cgggcccatgagactgacttcccactgctctgcctgcctctccccactgcactggcacagccccgccttgc
cgctgctgatccattgccggtgtgacccaagcacgcagcaatgcagctcaaaacgcttagcctagccacac
ccccacgggaaacagcagtgattaacctttagcaataaacgaaagtttaactaagctatactaaccccagg
gttggtcaatttcgtgccagccacacca
(nt, Anti-BCMA CAR full length ntd)
SEQ ID NO: 90
aggactcttctggtccccacagactcagagagaacccaccgccaccatggcactgccagtcacagcactgc
tgctgccactggcactgctgctccacgccgccagacccgagcagaagctgatcagcgaggaggacctggac
atccagatgacacagactacatcctccctgtctgcctctctgggagacagagtcaccatcagttgcagggc
aagtcaggacattagtaaatatttaaattggtatcagcagaaaccagatggaactgttaaactcctgatct
accatacatcaagattacactcaggagtcccatcaaggttcagtggcagtgggtctggaacagattattct
ctcaccattagcaacctggagcaagaagatattgccacttacttttgccaacagggtaatacgcttccgta
cacgttcggaggggggaccaagctggagatcacaggctccacaagcggcagcggaaagcccggaagcggcg
agggaagcaccaagggcgaggtgaaactgcaggagtcaggacctggcctggtggcgccctcacagagcctg
tccgtcacatgcactgtctcaggggtctcattacccgactatggtgtaagctggattcgccagcctccacg
aaagggtctggagtggctgggagtaatatggggtagtgaaaccacatactataattcagctctcaaatcca
gactgaccatcatcaaggacaactccaagagccaagttttcttaaaaatgaacagtctgcaaactgatgac
acagccatttactactgtgccaaacattattacgcaggtggtagctatgctatggactactggggccaagg
aacctcagtcaccgtctcctcattcgtgcccgtgttcctgccagccaagccaaccacaacaccagcaccca
gaccaccaacaccagcaccaacaatcgccagccagccactgtccctgaggccagaggcatgcagaccagca
gcaggcggagccgtgcacaccagaggactggacttcgcctgcgacatctacatctgggcaccactggccgg
aacatgcggcgtgctgctgctgagcctggtcatcaccctgtactgcaaccaccggaacaggtccgagagat
ctcgccttctccattctgactacatgaacatgacgccccgcaggccagggccaacacgagagcattatcag
ccatatgccccaccacgcgattttgctgcgtaccgctctagagtcgaattctcccggtccgctgacgcccc
tgcgtatcagcagggtcagaaccaactgtataatgagcttaatcttggaagaagagaggaatacgacgtat
tggatgagaggcgagggcgcgacccggaaatgggcggagaaccccagaggcgagagaatcctcaagaggga
ctttataacgagctgcaggaagatgagatggcggaagcatacagtgaaataggaatggaaggcgaacggcg
caggggcgaagggcatgatggtctgtatcagggtctttctactgcaacggaagacacgtatgacgccttgc
atatgcaggcgctgccccccaggtgatgcccgtcctcaccaagactgactgcctgctgctttgctactgcc
cgggcccatgagactgacttcccactgctctgcctgcctctccccactgcactggcacagccccgccttgc
cgctgctgatccattgccggtgtgacccaagcacgcagcaatgcagctcaaaacgcttagcctagccacac
ccccacgggaaacagcagtgattaacctttagcaataaacgaaagtttaactaagctatactaaccccagg
gttggtcaatttcgtgccagccacacca
(nt, Anti-BCMA CAR full length ntd)
SEQ ID NO: 91
aggactcttctggtccccacagactcagagagaacccaccgccaccatggcactgccagtcacagcactgc
tgctgccactggcactgctgctccacgccgccagacccgagcagaaactcatctcagaagaggatctggac
atccagatgacacagactacatcctccctgtctgcctctctgggagacagagtcaccatcagttgcagggc
aagtcaggacattagtaaatatttaaattggtatcagcagaaaccagatggaactgttaaactcctgatct
accatacatcaagattacactcaggagtcccatcaaggttcagtggcagtgggtctggaacagattattct
ctcaccattagcaacctggagcaagaagatattgccacttacttttgccaacagggtaatacgcttccgta
cacgttcggaggggggaccaagctggagatcacaggctccacaagcggcagcggaaagcccggaagcggcg
agggaagcaccaagggcgaggtgaaactgcaggagtcaggacctggcctggtggcgccctcacagagcctg
tccgtcacatgcactgtctcaggggtctcattacccgactatggtgtaagctggattcgccagcctccacg
aaagggtctggagtggctgggagtaatatggggtagtgaaaccacatactataattcagctctcaaatcca
gactgaccatcatcaaggacaactccaagagccaagttttcttaaaaatgaacagtctgcaaactgatgac
acagccatttactactgtgccaaacattattactacggtggtagctatgctatggactactggggccaagg
aacctcagtcaccgtctcctcattcgtgcccgtgttcctgccagccaagccaaccacaacaccagcaccca
gaccaccaacaccagcaccaacaatcgccagccagccactgtccctgaggccagaggcatgcagaccagca
gcaggcggagccgtgcacaccagaggactggacttcgcctgcgacatctacatctgggcaccactggccgg
aacatgcggcgtgctgctgctgagcctggtcatcaccctgtactgcaaccaccggaacaggagcaagaggt
ccaggctgctgcatagcgactacatgaacatgacccccagaaggcccggacccaccaggaagcactaccag
ccctacgccccccctagggattttgctgcctacaggtccagagtgaagttcagcagatccgccgacgcacc
agcctaccagcagggacagaaccagctgtacaacgagctgaacctggggagaagagaagagtacgacgtgc
tggataagcggagaggcagagaccctgagatgggcggcaagccccaaagacggaagaacccacaagagggc
ctgtacaacgagctgcagaaagacaagatggccgaggcctacagcgagatcggaatgaagggcgagcgcag
aagaggcaagggacacgacggactgtaccagggcctgagcacagccaccaaggatacctacgatgccctgc
acatgcaggccctgccaccaagatgatgcccgtcctcaccaagactgactgcctgctgctttgctactgcc
cgggcccatgagactgacttcccactgctctgcctgcctctccccactgcactggcacagccccgccttgc
cgctgctgatccattgccggtgtgacccaagcacgcagcaatgcagctcaaaacgcttagcctagccacac
ccccacgggaaacagcagtgattaacctttagcaataaacgaaagtttaactaagctatactaaccccagg
gttggtcaatttcgtgccagccacacca
(nt, Anti-BCMA CAR full length ntd)
SEQ ID NO: 92
aggactcttctggtccccacagactcagagagaacccaccgccaccatggcactgccagtcacagcactgc
tgctgccactggcactgctgctccacgccgccagacccgagcagaaactcatctcagaagaggatctggac
atccagatgacacagactacatcctccctgtctgcctctctgggagacagagtcaccatcagttgcagggc
aagtcaggacattagtaaatatttaaattggtatcagcagaaaccagatggaactgttaaactcctgatct
accatacatcaagattacactcaggagtcccatcaaggttcagtggcagtgggtctggaacagattattct
ctcaccattagcaacctggagcaagaagatattgccacttacttttgccaacagggtaatacgcttccgta
cacgttcggaggggggaccaagctggagatcacaggctccacaagcggcagcggaaagcccggaagcggcg
agggaagcaccaagggcgaggtgaaactgcaggagtcaggacctggcctggtggcgccctcacagagcctg
tccgtcacatgcactgtctcaggggtctcattacccgactatggtgtaagctggattcgccagcctccacg
aaagggtctggagtggctgggagtaatatggggtagtgaaaccacatactataattcagctctcaaatcca
gactgaccatcatcaaggacaactccaagagccaagttttcttaaaaatgaacagtctgcaaactgatgac
acagccatttactactgtgccaaacattattacgcaggtggtagctatgctatggactactggggccaagg
aacctcagtcaccgtctcctcattcgtgcccgtgttcctgccagccaagccaaccacaacaccagcaccca
gaccaccaacaccagcaccaacaatcgccagccagccactgtccctgaggccagaggcatgcagaccagca
gcaggcggagccgtgcacaccagaggactggacttcgcctgcgacatctacatctgggcaccactggccgg
aacatgcggcgtgctgctgctgagcctggtcatcaccctgtactgcaaccaccggaacaggagcaagaggt
ccaggctgctgcatagcgactacatgaacatgacccccagaaggcccggacccaccaggaagcactaccag
ccctacgccccccctagggattttgctgcctacaggtccagagtgaagttcagcagatccgccgacgcacc
agcctaccagcagggacagaaccagctgtacaacgagctgaacctggggagaagagaagagtacgacgtgc
tggataagcggagaggcagagaccctgagatgggcggcaagccccaaagacggaagaacccacaagagggc
ctgtacaacgagctgcagaaagacaagatggccgaggcctacagcgagatcggaatgaagggcgagcgcag
aagaggcaagggacacgacggactgtaccagggcctgagcacagccaccaaggatacctacgatgccctgc
acatgcaggccctgccaccaagatgatgcccgtcctcaccaagactgactgcctgctgctttgctactgcc
cgggcccatgagactgacttcccactgctctgcctgcctctccccactgcactggcacagccccgccttgc
cgctgctgatccattgccggtgtgacccaagcacgcagcaatgcagctcaaaacgcttagcctagccacac
ccccacgggaaacagcagtgattaacctttagcaataaacgaaagtttaactaagctatactaaccccagg
gttggtcaatttcgtgccagccacacca
(AA, Anti-BCMA CAR full length aa)
SEQ ID NO: 93
MALPVTALLLPLALLLHAARPDIVLTQSPPSLAMSLGKRATISCRASESVTILGSHLIHWYQQKPGQPPTL
LIQLASNVQTGVPARFSGSGSRTDFTLTIDPVEEDDVAVYYCLQSRTIPRTFGGGTKLEIKGSTSGSGKPG
SGEGSTKGQIQLVQSGPELKKPGETVKISCKASGYTFTDYSINWVKRAPGKGLKWMGWINTETREPAYAYD
FRGRFAFSLETSASTAYLQINNLKYEDTATYFCALDYSYAMDYWGQGTSVTVSSFVPVFLPAKPTTTPAPR
PPTPAPTIASQPLSLRPEACRPAAGGAVHTRGLDFACDIYIWAPLAGTCGVLLLSLVITLYCNHRNRSKRS
RLLHSDYMNMTPRRPGPTRKHYQPYAPPRDFAAYRSRVKFSRSADAPAYQQGQNQLYNELNLGRREEYDVL
DKRRGRDPEMGGKPRRKNPQEGLYNELQKDKMAEAYSEIGMKGERRRGKGHDGLYQGLSTATKDTYDALHM
QALPPR
(AA, Anti-BCMA CAR full length aa)
SEQ ID NO: 94
MALPVTALLLPLALLLHAARPDIQMTQSPSSLSASVGDRVTITCRASQGISNYLAWYQQKTGKVPKFLIYE
ASTLQSGVPSRFSGGGSGTDFTLTISSLQPEDVATYYCQNYNSAPFTFGPGTKVDIKGSTSGSGKPGSGEG
STKGQVQLVESGGGVVQPGRSLRLSCAASGFAFSRYGMHWVRQAPGKGLEWVAVIWYDGSNKYYADSVKGR
FTISRDNSKNTQYLQMNSLRAEDTAVYYCARGGDFLYYYYYGMDVWGQGTTVTVSSFVPVFLPAKPTTTPA
PRPPTPAPTIASQPLSLRPEACRPAAGGAVHTRGLDFACDIYIWAPLAGTCGVLLLSLVITLYCNHRNRVK
FSRSADAPAYQQGQNQLYNELNLGRREEYDVLDKRRGRDPEMGGKPRRKNPQEGLYNELQKDKMAEAYSEI
GMKGERRRGKGHDGLYQGLSTATKDTYDALHMQALPPR
(nt, Anti-BCMA CAR full length ntd)
SEQ ID NO: 95
aggactcttctggtccccacagactcagagagaacccaccgccgccaccatggccctgcccgtgacagctc
tgctgctccctctggctctgctgctgcacgctgccagacccgacatcgtgctgacacaaagcccccctagc
ctggccatgagcctgggcaagagggccacaatctcctgcagggccagcgagtccgtgaccatcctgggcag
ccacctgatccactggtaccagcagaagcctggccagccccctaccctgctgatccagctggcctccaacg
tgcagacaggagtgcccgctaggttctccggctccggcagcagaaccgactttaccctgaccatcgacccc
gtcgaggaggacgacgtggctgtctactattgcctgcagagcaggaccatccccaggaccttcggaggcgg
cacaaagctggagatcaagggcagcaccagcggctccggaaagcctggcagcggagaaggctccaccaagg
gacagattcagctggtgcagagcggacccgagctgaagaagcccggcgaaacagtcaagatcagctgcaaa
gcttccggctacaccttcacagactacagcatcaactgggtgaaaagggcccccggcaagggcctgaaatg
gatgggctggatcaacaccgagaccagggagcccgcctacgcctacgactttaggggcaggttcgccttca
gcctggagacatccgctagcaccgcttacctgcaaattaataacctgaagtacgaggacaccgccacctat
ttttgtgccctggactacagctacgcaatggactattggggacagggaacctccgtgaccgtgagcagctt
tgtgcccgtgttcctgcccgccaaacccaccaccacacctgctcctaggccccctacacctgcccctacca
tcgcttcccagcccctgagcctcagacctgaagcctgcagacctgctgctggaggcgccgtccacacaagg
ggactggacttcgcctgtgacatctacatctgggctcctctcgccggcacctgtggagtgctgctgctgtc
cctggtcatcaccctgtactgcaaccacaggaacaggagcaagaggtccaggctgctgcatagcgactaca
tgaacatgacccccagaaggcccggacccaccaggaagcactaccagccctacgccccccctagggatttt
gctgcctacaggtccagagtgaagttcagcagatccgctgatgcccctgcctatcagcagggccagaacca
actctacaacgagctgaacctgggaaggagggaggagtacgacgtgctcgacaagagaaggggaagggatc
ctgaaatgggcggcaagcccaggagaaagaaccctcaggagggcctgtacaacgaactgcagaaggataag
atggccgaggcctacagcgaaatcggcatgaaaggcgagagaaggagaggcaagggccacgacggactgta
ccagggcctgtccaccgccaccaaagacacctacgatgccctgcacatgcaggccctgccccccaggtgat
gcccgtcctcaccaagactgactgcctgctgctttgctactgcccgggcccatgagactgacttcccactg
ctctgcctgcctctccccactgcactggcacagccccgccttgccgctgctgatccattgccggtgtgacc
caagcacgcagcaatgcagctcaaaacgcttagcctagccacacccccacgggaaacagcagtgattaacc
tttagcaataaacgaaagtttaactaagctatactaaccccagggttggtcaatttcgtgccagccacacc
a
(nt, Anti-BCMA CAR full length ntd)
SEQ ID NO: 96
aggactcttctggtccccacagactcagagagaacccaccgccgccaccatggccctgcccgtgacagctc
tgctgctccctctggctctgctgctgcacgctgccagacccgacatcgtgctgacacaaagcccccctagc
ctggccatgagcctgggcaagagggccacaatctcctgcagggccagcgagtccgtgaccatcctgggcag
ccacctgatccactggtaccagcagaagcctggccagccccctaccctgctgatccagctggcctccaacg
tgcagacaggagtgcccgctaggttctccggctccggcagcagaaccgactttaccctgaccatcgacccc
gtcgaggaggacgacgtggctgtctactattgcctgcagagcaggaccatccccaggaccttcggaggcgg
cacaaagctggagatcaagggcagcaccagcggctccggaaagcctggcagcggagaaggctccaccaagg
gacagattcagctggtgcagagcggacccgagctgaagaagcccggcgaaacagtcaagatcagctgcaaa
gcttccggctacaccttcacagactacagcatcaactgggtgaaaagggcccccggcaagggcctgaaatg
gatgggctggatcaacaccgagaccagggagcccgcctacgcctacgactttaggggcaggttcgccttca
gcctggagacatccgctagcaccgcttacctgcaaattaataacctgaagtacgaggacaccgccacctat
ttttgtgccctggactacagctacgcaatggactattggggacagggaacctccgtgaccgtgagcagctt
tgtgcccgtgttcctgcccgccaaacccaccaccacacctgctcctaggccccctacacctgcccctacca
tcgcttcccagcccctgagcctcagacctgaagcctgcagacctgctgctggaggcgccgtccacacaagg
ggactggacttcgcctgtgacatctacatctgggctcctctcgccggcacctgtggagtgctgctgctgtc
cctggtcatcaccctgtactgcaaccacaggaacagagtgaagttcagcagatccgctgatgcccctgcct
atcagcagggccagaaccaactctacaacgagctgaacctgggaaggagggaggagtacgacgtgctcgac
aagagaaggggaagggatcctgaaatgggcggcaagcccaggagaaagaaccctcaggagggcctgtacaa
cgaactgcagaaggataagatggccgaggcctacagcgaaatcggcatgaaaggcgagagaaggagaggca
agggccacgacggactgtaccagggcctgtccaccgccaccaaagacacctacgatgccctgcacatgcag
gccctgccccccaggtgatgcccgtcctcaccaagactgactgcctgctgctttgctactgcccgggccca
tgagactgacttcccactgctctgcctgcctctccccactgcactggcacagccccgccttgccgctgctg
atccattgccggtgtgacccaagcacgcagcaatgcagctcaaaacgcttagcctagccacacccccacgg
gaaacagcagtgattaacctttagcaataaacgaaagtttaactaagctatactaaccccagggttggtca
atttcgtgccagccacacca
(nt, Anti-BCMA CAR full length ntd)
SEQ ID NO: 97
aggactcttctggtccccacagactcagagagaacccaccgccaccatggcactgccagtcacagcactgc
tgctgccactggcactgctgctccacgccgccagacccgacatcgtgctgacacaaagcccccctagcctg
gccatgagcctgggcaagagggccacaatctcctgcagggccagcgagtccgtgaccatcctgggcagcca
cctgatccactggtaccagcagaagcctggccagccccctaccctgctgatccagctggcctccaacgtgc
agacaggagtgcccgctaggttctccggctccggcagcagaaccgactttaccctgaccatcgaccccgtc
gaggaggacgacgtggctgtctactattgcctgcagagcaggaccatccccaggaccttcggaggcggcac
aaagctggagatcaagggcagcaccagcggctccggaaagcctggcagcggagaaggctccaccaagggac
agattcagctggtgcagagcggacccgagctgaagaagcccggcgaaacagtcaagatcagctgcaaggcc
tccggctacaccttcacagactacagcatcaactgggtgaaaagggcccccggcaagggcctgaaatggat
gggctggatcaacaccgagaccagggagcccgcctacgcctacgactttaggggcaggttcgccttcagcc
tggagacatccgctagcaccgcttacctgcaaattaataacctgaagtacgaggacaccgccacctatttt
tgtgccctggactacagctacgcaatggactattggggacagggaacctccgtgaccgtgagcagcttcgt
gcccgtgttcctgccagccaagccaaccacaacaccagcacccagaccaccaacaccagcaccaacaatcg
ccagccagccactgtccctgaggccagaggcatgcagaccagcagcaggcggagccgtgcacaccagagga
ctggacttcgcctgcgacatctacatctgggcaccactggccggaacatgcggcgtgctgctgctgagcct
ggtcatcaccctgtactgcaaccaccggaacaggtccgagagatctcgccttctccattctgactacatga
acatgacgccccgcaggccagggccaacacgagagcattatcagccatatgccccaccacgcgattttgct
gcgtaccgctctagagtcgaattctcccggtccgctgacgcccctgcgtatcagcagggtcagaaccaact
gtataatgagcttaatcttggaagaagagaggaatacgacgtattggatgagaggcgagggcgcgacccgg
aaatgggcggagaaccccagaggcgagagaatcctcaagagggactttataacgagctgcaggaagatgag
atggcggaagcatacagtgaaataggaatggaaggcgaacggcgcaggggcgaagggcatgatggtctgta
tcagggtctttctactgcaacggaagacacgtatgacgccttgcatatgcaggcgctgccccccaggtgat
gcccgtcctcaccaagactgactgcctgctgctttgctactgcccgggcccatgagactgacttcccactg
ctctgcctgcctctccccactgcactggcacagccccgccttgccgctgctgatccattgccggtgtgacc
caagcacgcagcaatgcagctcaaaacgcttagcctagccacacccccacgggaaacagcagtgattaacc
tttagcaataaacgaaagtttaactaagctatactaaccccagggttggtcaatttcgtgccagccacacc
a
(nt, Anti-BCMA CAR full length ntd)
SEQ ID NO: 98
aggactcttctggtccccacagactcagagagaacccaccgccaccatggcactgccagtcacagcactgc
tgctgccactggcactgctgctccacgccgccagacccgacatcgtgctgacacagagcccagcaagcctg
gccgtgagcccaggccagagagccacaatcacatgcagagccagcgagtccgtgtccttcctgggcatcaa
cctgatccactggtaccagcagaagcccggacagccacccaaactgctgatctactccgccagcaacctgc
agtcaggcgtgccagccagattcagcggcagcggcagcggaaccgacttcaccctgacaatcagctcagtg
gaaccagaagacaccgccaactactactgcctgcagagcagaaccctgcccagaaccttcggccaaggcac
caaggtcgaaatcaagggctccacaagcggcagcggaaagcccggaagcggcgagggaagcaccaagggcc
agatccagctggtgcagagcggccccgaactgaagaaacccggcggaagcgtgaaaatctcctgcaaggcc
agcggctacaccttcacctcctacagcatcaactgggtgcgccaggccccaggcaagggcctggaatgggt
cggctggatcaacaccgagacaagagagcccgcctacgcccagggcttcacaggcagattcaccttcagcg
cagacacaagcaagagcatggcctacctgcagatcaactccctgagagcagaggacaccgccgtctactac
tgcgccctggactacctctactccctggacttctggggccagggcaccctcgtgaccgtgtccagcttcgt
gcccgtgttcctgccagccaagccaaccacaacaccagcacccagaccaccaacaccagcaccaacaatcg
ccagccagccactgtccctgaggccagaggcatgcagaccagcagcaggcggagccgtgcacaccagagga
ctggacttcgcctgcgacatctacatctgggcaccactggccggaacatgcggcgtgctgctgctgagcct
ggtcatcaccctgtactgcaaccaccggaacagagtcgaattctcccggtccgctgacgcccctgcgtatc
agcagggtcagaaccaactgtataatgagcttaatcttggaagaagagaggaatacgacgtattggatgag
aggcgagggcgcgacccggaaatgggcggagaaccccagaggcgagagaatcctcaagagggactttataa
cgagctgcaggaagatgagatggcggaagcatacagtgaaataggaatggaaggcgaacggcgcaggggcg
aagggcatgatggtctgtatcagggtctttctactgcaacggaagacacgtatgacgccttgcatatgcag
gcgctgccccccaggtgatgcccgtcctcaccaagactgactgcctgctgctttgctactgcccgggccca
tgagactgacttcccactgctctgcctgcctctccccactgcactggcacagccccgccttgccgctgctg
atccattgccggtgtgacccaagcacgcagcaatgcagctcaaaacgcttagcctagccacacccccacgg
gaaacagcagtgattaacctttagcaataaacgaaagtttaactaagctatactaaccccagggttggtca
atttcgtgccagccacacca
(nt, Anti-BCMA CAR full length ntd)
SEQ ID NO: 99
aggactcttctggtccccacagactcagagagaacccaccgccaccatggcactgccagtcacagcactgc
tgctgccactggcactgctgctccacgccgccagacccgacatcgtgctgacacagagcccagcaagcctg
gccgtgagcccaggccagagagccacaatcacatgcagagccagcgagtccgtgtccttcctgggcatcaa
cctgatccactggtaccagcagaagcccggacagccacccaaactgctgatctactccgccagcaacctgc
agtcaggcgtgccagccagattcagcggcagcggcagcggaaccgacttcaccctgacaatcagctcagtg
gaaccagaagacaccgccaactactactgcctgcagagcagaaccctgcccagaaccttcggccaaggcac
caaggtcgaaatcaagggctccacaagcggcagcggaaagcccggaagcggcgagggaagcaccaagggcc
agatccagctggtgcagagcggccccgaactgaagaaacccggcggaagcgtgaaaatctcctgcaaggcc
agcggctacaccttcacctcctacagcatcaactgggtgcgccaggccccaggcaagggcctggaatgggt
cggctggatcaacaccgagacaagagagcccgcctacgcccagggcttcacaggcagattcaccttcagcg
cagacacaagcaagagcatggcctacctgcagatcaactccctgagagcagaggacaccgccgtctactac
tgcgccctggactacctctactccctggacttctggggccagggcaccctcgtgaccgtgtccagcttcgt
gcccgtgttcctgccagccaagccaaccacaacaccagcacccagaccaccaacaccagcaccaacaatcg
ccagccagccactgtccctgaggccagaggcatgcagaccagcagcaggcggagccgtgcacaccagagga
ctggacttcgcctgcgacatctacatctgggcaccactggccggaacatgcggcgtgctgctgctgagcct
ggtcatcaccctgtactgcaaccaccggaacaggtccgagagatctcgccttctccattctgactacatga
acatgacgccccgcaggccagggccaacacgagagcattatcagccatatgccccaccacgcgattttgct
gcgtaccgctctagagtcgaattctcccggtccgctgacgcccctgcgtatcagcagggtcagaaccaact
gtataatgagcttaatcttggaagaagagaggaatacgacgtattggatgagaggcgagggcgcgacccgg
aaatgggcggagaaccccagaggcgagagaatcctcaagagggactttataacgagctgcaggaagatgag
atggcggaagcatacagtgaaataggaatggaaggcgaacggcgcaggggcgaagggcatgatggtctgta
tcagggtctttctactgcaacggaagacacgtatgacgccttgcatatgcaggcgctgccccccaggtgat
gcccgtcctcaccaagactgactgcctgctgctttgctactgcccgggcccatgagactgacttcccactg
ctctgcctgcctctccccactgcactggcacagccccgccttgccgctgctgatccattgccggtgtgacc
caagcacgcagcaatgcagctcaaaacgcttagcctagccacacccccacgggaaacagcagtgattaacc
tttagcaataaacgaaagtttaactaagctatactaaccccagggttggtcaatttcgtgccagccacacc
a
(nt, Anti-BCMA CAR full length ntd)
SEQ ID NO: 100
aggactcttctggtccccacagactcagagagaacccaccgccaccatggccctgcccgtgacagctctgc
tgctccctctggctctgctgctgcacgctgccagacccgacatcgtgctgacacaaagcccccctagcctg
gccatgagcctgggcaagagggccacaatctcctgcagggccagcgagtccgtgaccatcctgggcagcca
cctgatccactggtaccagcagaagcctggccagccccctaccctgctgatccagctggcctccaacgtgc
agacaggagtgcccgctaggttctccggctccggcagcagaaccgactttaccctgaccatcgaccccgtc
gaggaggacgacgtggctgtctactattgcctgcagagcaggaccatccccaggaccttcggaggcggcac
aaagctggagatcaagggcagcaccagcggctccggaaagcctggcagcggagaaggctccaccaagggac
agattcagctggtgcagagcggacccgagctgaagaagcccggcgaaacagtcaagatcagctgcaaagct
tccggctacaccttcacagactacagcatcaactgggtgaaaagggcccccggcaagggcctgaaatggat
gggctggatcaacaccgagaccagggagcccgcctacgcctacgactttaggggcaggttcgccttcagcc
tggagacatccgctagcaccgcttacctgcaaattaataacctgaagtacgaggacaccgccacctatttt
tgtgccctggactacagctacgcaatggactattggggacagggaacctccgtgaccgtgagcagcttcgt
gcccgtgttcctgccagccaagccaaccacaacaccagcacccagaccaccaacaccagcaccaacaatcg
ccagccagccactgtccctgaggccagaggcatgcagaccagcagcaggcggagccgtgcacaccagagga
ctggacttcgcctgcgacatctacatctgggcaccactggccggaacatgcggcgtgctgctgctgagcct
ggtcatcaccctgtactgcaaccaccggaacagagtcgaattctcccggtccgctgacgcccctgcgtatc
agcagggtcagaaccaactgtataatgagcttaatcttggaagaagagaggaatacgacgtattggatgag
aggcgagggcgcgacccggaaatgggcggagaaccccagaggcgagagaatcctcaagagggactttataa
cgagctgcaggaagatgagatggcggaagcatacagtgaaataggaatggaaggcgaacggcgcaggggcg
aagggcatgatggtctgtatcagggtctttctactgcaacggaagacacgtatgacgccttgcatatgcag
gcgctgccccccaggtgatgcccgtcctcaccaagactgactgcctgctgctttgctactgcccgggccca
tgagactgacttcccactgctctgcctgcctctccccactgcactggcacagccccgccttgccgctgctg
atccattgccggtgtgacccaagcacgcagcaatgcagctcaaaacgcttagcctagccacacccccacgg
gaaacagcagtgattaacctttagcaataaacgaaagtttaactaagctatactaaccccagggttggtca
atttcgtgccagccacacca
(AA, Anti-BCMA CAR full length)
SEQ ID NO: 101
MALPVTALLLPLALLLHAARPDIVLTQSPASLAVSPGQRATITCRASESVSFLGINLIHWYQQKPGQPPKL
LIYSASNLQSGVPARFSGSGSGTDFTLTISSVEPEDTANYYCLQSRTLPRTFGQGTKVEIKGSTSGSGKPG
SGEGSTKGQIQLVQSGPELKKPGGSVKISCKASGYTFTSYSINWVRQAPGKGLEWVGWINTETREPAYAQG
FTGRFTFSADTSKSMAYLQINSLRAEDTAVYYCALDYLYSLDFWGQGTLVTVSSFVPVFLPAKPTTTPAPR
PPTPAPTIASQPLSLRPEACRPAAGGAVHTRGLDFACDIYIWAPLAGTCGVLLLSLVITLYCNHRNRVEFS
RSADAPAYQQGQNQLYNELNLGRREEYDVLDERRGRDPEMGGEPQRRENPQEGLYNELQEDEMAEAYSEIG
MEGERRRGEGHDGLYQGLSTATEDTYDALHMQALPPR
(AA, Anti-BCMA CAR full length)
SEQ ID NO: 102
MALPVTALLLPLALLLHAARPDIVLTQSPASLAVSPGQRATITCRASESVSFLGINLIHWYQQKPGQPPKL
LIYSASNLQSGVPARFSGSGSGTDFTLTISSVEPEDTANYYCLQSRTLPRTFGQGTKVEIKGSTSGSGKPG
SGEGSTKGQIQLVQSGPELKKPGGSVKISCKASGYTFTSYSINWVRQAPGKGLEWVGWINTETREPAYAQG
FTGRFTFSADTSKSMAYLQINSLRAEDTAVYYCALDYLYSLDFWGQGTLVTVSSFVPVFLPAKPTTTPAPR
PPTPAPTIASQPLSLRPEACRPAAGGAVHTRGLDFACDIYIWAPLAGTCGVLLLSLVITLYCNHRNRSERS
RLLHSDYMNMTPRRPGPTREHYQPYAPPRDFAAYRSRVEFSRSADAPAYQQGQNQLYNELNLGRREEYDVL
DERRGRDPEMGGEPQRRENPQEGLYNELQEDEMAEAYSEIGMEGERRRGEGHDGLYQGLSTATEDTYDALH
MQALPPR

1. A protein that is capable of intracellular signaling comprising an intracellular domain, wherein the intracellular domain comprises an intracellular signaling domain, a costimulatory domain, or both, and wherein at least two lysine amino acids of the intracellular domain are substituted by an amino acid independently selected from the group consisting of alanine, aspartate, asparagine, glutamate, glutamine, histidine, leucine, methionine, serine, threonine, and valine.

2. The protein of claim 1, wherein the intracellular domain comprises a CD3-zeta domain.

3. The protein of claims 1 and 2, wherein the intracellular domain comprises a domain that is at least 80%, at least 85%, at least 90%, at least 95%, or at least 99% identical to SEQ ID NO: 18, 61, or 66.

4. The protein of any one of claims 1-3, where the CD3-zeta intracellular domain is 100% identical to SEQ ID NO:18, 61, or 66 except for the substituted lysine amino acids.

5. The protein of any one of claims 1-4, wherein at least three lysine amino acids are substituted by an amino acid independently selected from the group consisting of alanine, aspartate, asparagine, glutamate, glutamine, histidine, leucine, methionine, serine, threonine, and valine.

6. The protein of any one of claims 1-5, wherein at least four, at least five, at least six, at least seven, or at least eight lysine amino acids are substituted by an amino acid independently selected from the group consisting of alanine, aspartate, asparagine, glutamate, glutamine, histidine, leucine, methionine, serine, threonine, and valine.

7. The protein of any one of claims 1-6, wherein at least nine lysine amino acids are substituted by an amino acid independently selected from the group consisting of alanine, aspartate, asparagine, glutamate, glutamine, histidine, leucine, methionine, serine, threonine, and valine.

8. The protein of any one of claims 1-7, wherein at least six at least seven, at least eight, or at least nine lysine amino acids are substituted by an amino acid independently selected from the group consisting of alanine, aspartate, and glutamate.

9. A CD3-zeta intracellular domain, wherein at least two lysine amino acids of the CD3-zeta intracellular domain are substituted by an amino acid independently selected from the group consisting of alanine, aspartate, asparagine, glutamate, glutamine, histidine, leucine, methionine, serine, threonine, and valine.

10. The CD3-zeta intracellular domain of claim 9, wherein the intracellular domain comprises a domain that is at least 80%, at least 85%, at least 90%, at least 95%, or at least 99% identical to SEQ ID NO:18 or 61.

11. The CD3-zeta intracellular domain of claim 9 or 10, wherein the CD3-zeta intracellular domain is 100% identical to SEQ ID NO:18 or 61 except for the substituted lysine amino acids.

12. The CD3-zeta intracellular domain of any one of claims 9-11, wherein the substituted lysine amino acids are selected from the group consisting of positions 3, 37, 48, 53, 65, 67, 78, 85, and 99 of the amino acid sequence of SEQ ID NO: 18.

13. The CD3-zeta intracellular domain of any of claims 9-12, wherein at least three lysine amino acids are substituted by an amino acid independently selected from the group consisting of alanine, aspartate, asparagine, glutamate, glutamine, histidine, leucine, methionine, serine, threonine, and valine.

14. The CD3-zeta intracellular domain of any one of claims 9-13, wherein at least four, at least five, at least six, at least seven, or at least eight lysine amino acids are substituted by an amino acid independently selected from the group consisting of alanine, aspartate, asparagine, glutamate, glutamine, histidine, leucine, methionine, serine, threonine, and valine.

15. The CD3-zeta intracellular domain of any one of claims 9-14, wherein at least nine lysine amino acids are substituted by an amino acid independently selected from the group consisting of alanine, aspartate, asparagine, glutamate, glutamine, histidine, leucine, methionine, serine, threonine, and valine.

16. The CD3-zeta intracellular domain of any one of claims 9-15, wherein at least six at least seven, at least eight, or at least nine lysine amino acids are substituted by an amino acid independently selected from the group consisting of alanine, aspartate, and glutamate.

17. The CD3-zeta intracellular domain of any of claims 9-16, wherein the CD3-zeta intracellular domain is a domain of a protein.

18. The CD3-zeta intracellular domain of any of claims 9-17, wherein the CD3-zeta intracellular domain is a domain of a transmembrane protein.

19. The CD3-zeta intracellular domain of any of claims 9-18, wherein the CD3-zeta intracellular domain is a domain of an intercellular signaling protein.

20. The CD3-zeta intracellular domain of any of claims 9-19, wherein the CD3-zeta intracellular domain is a domain of a CAR.

21. A protein comprising the CD3-zeta intracellular domain of any of claims 9-20.

22. A protein comprising a CD3-zeta intracellular domain, wherein the CD3-zeta intracellular domain is at least 80%, at least 85%, at least 90%, at least 95%, or at least 99% identical to SEQ ID NO:18, wherein at least two lysine amino acids of SEQ ID NO: 18 are either (a) deleted or (b) substituted by an amino acid independently selected from the group consisting of alanine, aspartate, asparagine, glutamate, glutamine, histidine, leucine, methionine, serine, threonine, and valine.

23. The protein of claim 22, where the CD3-zeta intracellular domain is 100% identical to SEQ ID NO:18 except for the deleted or substituted lysine amino acids.

24. The protein of claim 22 or 23, wherein at least three, at least four, at least five, at least six, at least seven, at least eight, or at least nine lysine amino acids of SEQ ID NO: 18 are either (a) deleted or (b) substituted by an amino acid independently selected from the group consisting of alanine, aspartate, and glutamate.

25. The protein of any one of claims 22-24, wherein at least three, at least four, at least five, at least six, at least seven, at least eight, or at least nine lysine amino acids of SEQ ID NO: 18 are substituted by alanine.

26. The protein of any one of claims 22-25, wherein at least three, at least four, at least five, at least six, at least seven, at least eight, or at least nine lysine amino acids of SEQ ID NO: 18 are substituted by aspartate.

27. The protein of any one of claims 22-26, wherein at least three, at least four, at least five, at least six, at least seven, at least eight, or at least nine lysine amino acids of SEQ ID NO: 18 are substituted by glutamate.

28. A protein of claim 22 comprising an amino acid sequence that is at least 80% identical to any one of SEQ ID NOs: 2-7, 19-66, and 68-86.

29. The protein of claim 22 comprising an amino acid sequence that is, at least 85%, at least 90%, at least 95%, or at least 99% identical to any one of SEQ ID NOs: 2-7, 19-66, and 68-86.

30. The protein of any one of claim 1-8 or 22-29, wherein the protein is a Chimeric Antigen Receptor (CAR).

31. The protein of any one of claim 1-8 or 22-29, further comprising a costimulatory domain.

32. The protein of claim 31, wherein the costimulatory domain is selected from the group consisting of CD8-alpha domains, 41BB domains, CD28 domains, FcR gamma domains, CD27 domains, OX40 domains, CD30 domains, CD40 domains, PD-1 domains, ICOS domains, LFA-1 domains, CD2 domains, CD7 domains, LIGHT domains, NKG2C domains, and B7 H3 domains, and any variants thereof.

33. The protein of claim 31, wherein the costimulatory domain is CD28.

34. The protein of claim 31, wherein the costimulatory domain is 41BB.

35. The protein of any one of claims 31-34, wherein at least one lysine amino acids of the costimulatory domain are substituted by an amino acid independently selected from the group consisting of alanine, aspartate, asparagine, glutamate, glutamine, histidine, leucine, methionine, serine, threonine, and valine.

36. The protein of any one of claims 31-35, wherein at least two lysine amino acids of the costimulatory domain are substituted by an amino acid independently selected from the group consisting of alanine, aspartate, and glutamate.

37. The protein of any one of claim 1-8 or 22-36, further comprising an extracellular antigen binding domain.

38. The protein of claim 37, wherein the extracellular antigen binding domain binds CD19, BCMA, EGFR/HER, CD22, mesothelin, CD123, CD20, PD1, or CD30.

39. The protein of claim 37 or 38, wherein the extracellular antigen binding domain binds BCMA.

40. The protein of claim 37 or 38, wherein the extracellular antigen binding domain binds CD19.

41. The protein of any of claims 37-40, wherein the extracellular antigen binding domain is a scFv.

42. The protein of any one of claim 1-8 or 22-41, further comprising a transmembrane domain.

43. The protein of claim 42, wherein the transmembrane domain comprises the transmembrane region of MHC class I molecules, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, or a receptor that specifically binds with CD83.

44. The protein of any one of claim 1-8 or 22-43, further comprising a hinge region.

45. The protein of any one of claim 1-8 or 22-44, further comprising a leader domain.

46. The protein of any one of claims 31-45, further comprises one or more spacer sequences between one or more of the domains.

47. The protein of claim 46, wherein the spacer sequences is a polypeptide linker.

48. The CD3-zeta intracellular domain of claim 9 comprising any one of sequences SEQ ID NO: 28-56 or 69-86.

49. The protein of claim 22 comprising any one of sequences SEQ ID NO: 57-60 and 62-65.

50. A protein comprising the sequence of SEQ ID NO: 5, 68, 101, or 102.

51. A nucleic acid construct encoding the protein of any one of claim 1-8 or 22-50 or the CD3-zeta intracellular domain of any one of claim 9-21.

52. The nucleic acid construct of claim 51, wherein the nucleic acid construct is RNA.

53. The nucleic acid construct of claim 51, wherein the nucleic acid construct is DNA.

54. A nucleic acid construct comprising SEQ ID NOs. 13-17 or 67.

55. A vector encoding the protein of any one of claim 1-8 or 22-50 or the CD3-zeta intracellular domain of any one of claim 9-21.

56. A vector comprising the nucleic acid construct of any one of claims 51-54.

57. The vector of claim 55 or 56, wherein the vector is a viral vector.

58. A composition comprising the protein of any one of claim 1-8 or 22-50, the CD3-zeta intracellular domain of any one of claim 9-21, the nucleic acid construct of any one of claims 51-54, or the vector of any one of claims 55-57.

59. A pharmaceutical composition comprising the composition of claim 58.

60. The pharmaceutical composition of claim 59, further comprising a pharmaceutically acceptable excipient.

61. A cell comprising the protein of any one of claim 1-8 or 22-50.

62. A cell comprising the nucleic acid construct of any one of claims 51-54 or the vector of any one of claims 55-57.

63. The cell of claim 61 or 62, wherein the cell is a human cell.

64. The cell of any one of claims 61-63, wherein the cell is an immune cell.

65. The cell of any one of claims 61-63, wherein the cell is a T-cell, CD3+ cell, CD8+ cell, CD4+ cell, NK cell, stem cell, hematopoietic stem cell, or mesenchymal stem cell.

66. A method for producing a cell therapy for treating a disease, the method comprising transfecting a plurality of cells with a vector of claim 55 or 56.

67. A method of transfecting a cell with a plurality of cells with the vector of any one of claims 55-57.

68. The method of claim 67, wherein the method is in vitro.

69. The method of claim 67, wherein the method is ex vivo.

70. The method of claim 67, wherein the method is in vivo.

71. A method of treating a disease in a subject in need thereof, the method comprising administering to the subject a cell of any one of claims 61-65.

72. A method of transfecting a cell with the vector of any one of claims 55-57.

73. The method of claim 72, wherein the of cell is a human cell.

74. The method of claim 72 or 73, wherein the cell is an immune cell.

75. The method of any one of claims 72-74, wherein the cells are T cells.

76. The method of any one of claims 72-74, wherein the cells are CD3+ cells.

77. The method of any one of claims 72-74, wherein the cells are CD8+ cells.

78. The method of any one of claims 72-74, wherein the cells are CD4+ cells.

79. The method of any one of claims 72-74, wherein the cells are NK cells.

80. The method of any one of claims 72-74, wherein the cells are stem cells.

81. The method of claim 80, wherein the stem cells are hematopoietic stem cells.

82. The method of claim 80, wherein the stem cells are mesenchymal stem cells.

83. The method any one of claim 71 or 73-82, further comprising administering a cytokine.

84. The method of any one of claim 71 or 73-83, wherein the disease is cancer, an autoimmune condition, or an allergic condition.

85. The method of any one of claim 71 or 73-84, wherein the disease is a cancer.

86. The method of any one of claim 71 or 73-84, wherein the disease is a myeloma.

87. The method of any one of claim 71 or 73-84, wherein the disease myasthenia gravis (MG) disease

88. The method of any one of claim 71 or 73-87, wherein the method is characterized by increased cellular secretion of a cytokine.

89. The method of claim 88, wherein the secreted cytokine is interferon gamma.

90. The method of any one of claim 71 or 73-89, wherein the method is characterized by the selective killing of cancer cells.

91. The method of any one of claim 71 or 73-89, wherein the method is characterized by the selective killing of immune cells.

92. The method of any one of claim 71 or 73-89, wherein the method is characterized by the selective killing of BCMA+ or CD19+ cells.

93. Use of the protein of any one of claim 1-8 or 22-50, the CD3-zeta intracellular domain of any one of claim 9-21, the nucleic acid construct of any one of claims 51-54, the vector of any one of claims 55-57, the composition of claim 58, the pharmaceutical composition of claim 59 or 60, or the cell of any one of claims 61-65 for the treatment of cancer.

94. A kit comprising one or more of the proteins of any one of claim 1-8 or 22-50, the CD3-zeta intracellular domain of any one of claim 9-21, the nucleic acid construct of any one of claims 51-54, the vector of any one of claims 55-57, the composition of claim 58, the pharmaceutical composition of claim 59 or 60, or the cell of any one of claims 61-65 for the treatment of cancer.
